(19) United States
(12) Patent Application Publication
(10) Pub. No.: US 2012/0214847 A1
(43) Pub. Date: Aug. 23, 2012

(54) 2-[1-PHENYL-5-HYDROXY-4a-SUBSTITUTED-HEXAHYDROCYCLOPENTA[F]INDAZOL-5-YL]ETHYL PHENYL DERIVATIVES AS GLUCOCORTICOID RECEPTOR LIGANDS

(75) Inventors: Christopher James Bungard, Lansdale, PA (US); James J. Perkins, Churchville, PA (US); Jesse J. Manikowski, Norristown, PA (US); Pablo de Leon, Philadelphia, PA (US); Robert S. Meissner, Schwenksville, PA (US)

(73) Assignee: Merck Sharp & Dohme Corp., Rahway, NJ (US)

(21) Appl. No.: 13/504,260

(22) PCT Filed: Oct. 26, 2010
(86) PCT No.: PCT/US10/54052

§ 371 Date: Apr. 26, 2012

Related U.S. Application Data
(60) Provisional application No. 61/256,676, filed on Oct. 30, 2009.

(51) Int. Cl. A61K 31/4439; C07D 401/10; C07D 231/54; A61P 41/00; A61P 35/02; A61P 37/00; A61P 3/10; A61P 3/04; A61P 3/00; A61P 1/00; A61P 19/02; A61P 37/08; A61P 11/00; A61P 11/06; A61P 21/00; A61P 31/14; A61P 29/00; A61P 17/06; A61P 17/00; A61P 17/10; A61P 31/18; A61P 35/00; A61P 25/00; A61P 25/24; A61P 25/18; A61P 25/20; A61P 25/22; C07D 401/04; A61K 31/416

(52) U.S. Cl. 514/338; 514/406; 546/275.7; 548/356.5

(57) ABSTRACT

The present invention encompasses compounds of Formula I: or pharmaceutically acceptable salts or hydrates thereof, which are useful as selective glucocorticoid receptor ligands for treating a variety of autoimmune and inflammatory diseases or conditions. Pharmaceutical compositions and methods of use are also included.

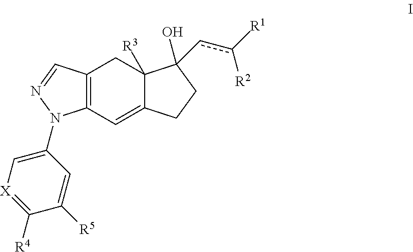

### BACKGROUND OF THE INVENTION

Intracellular receptors (IR's) are a class of structurally related proteins involved in the regulation of gene expression. The steroid hormone receptors are a subset of this superfamily whose natural ligands are typically comprised of endogenous steroids such as estradiol, progesterone, and cortisol. Man-made ligands to these receptors play an important role in human health and, of these receptors, the glucocorticoid receptor has an essential role in regulating human physiology and immune response. Steroids that interact with the glucocorticoid receptor have been shown to be potent anti-inflammatory agents. The present invention is directed to a novel class of compounds that are selective glucocorticoid receptor modulators that have potent anti-inflammatory and immunosuppressive activity and possess advantages over steroidal glucocorticoid ligands with respect to side effects, efficacy, toxicity and/or metabolism.

### SUMMARY OF THE INVENTION

The present invention encompasses compounds of Formula I:

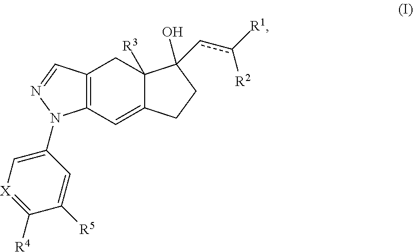

or pharmaceutically acceptable salts or hydrates thereof, which are useful as selective glucocorticoid receptor ligands for treating a variety of autoimmune and inflammatory diseases or conditions. Pharmaceutical compositions and methods of use are also included.

### DETAILED DESCRIPTION OF THE INVENTION

The present invention encompasses a compound of Formula I,

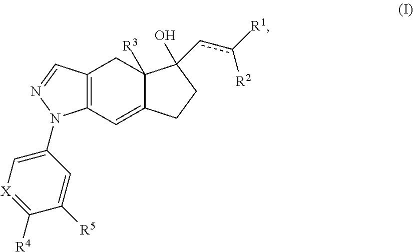

or a pharmaceutically acceptable salt thereof, or a stereoisomer thereof, or a pharmaceutically acceptable salt of the stereoisomer thereof:
wherein
“ [symbol] ” is an optional double bond;

### X is —CH═ or —N═;

R¹ is selected from the group consisting of:
(1) aryl, and
(2) HET;
wherein each of items (1) and (2) is optionally substituted with one to three groups independently selected from:
   (a) halogen,
   (b) —OR^{a},
   (c) —C(O)—R^{a},
   (d) —C(O)—OR^{a},
   (e) —C(O)NR^{a}R^{b},
   (f) C₁₋₆alkyl, optionally substituted with one to three groups selected from halogen, hydroxy, and —C(O)NR^{a}R^{b},
   (g) C₃₋₆cycloalkyl,
   (h) —SO₂R^{a}, and
   (i) nitrile;
      R² is selected from the group consisting of:
(1) hydrogen,
(2) C₁₋₆alkyl, and
(3) hydroxy;
   R³ is selected from the group consisting of:
(1) C₁₋₆alkyl, optionally substituted with one to three halogens, and
(2) C₃₋₆cycloalkyl;
   each of R⁴ and R⁵ is independently selected from the group consisting of:
(1) hydrogen,
(2) C₁₋₆alkyl, and
(3) halogen; and
   each of R^{a} and R^{b} is independently selected from the group consisting of:
(1) hydrogen,
(2) C₁₋₆alkyl, optionally substituted with one to three groups selected from halogen, hydroxy, C₃₋₆cycloalkyl, and —C(O)NH₂, and
(3) C₃₋₆cycloalkyl, optionally substituted with one to three groups selected from halogen, C₁₋₆alkyl, and C₁₋₆alkoxy.

In one embodiment, aryl is selected from phenyl and naphthyl. In another embodiment, aryl is phenyl.

In one embodiment, HET is a 5- or 6-membered aromatic or non-aromatic monocyclic ring containing 1-3 heteroatoms selected from O, S and N. In another embodiment, HET is a 9- or 10-membered aromatic or partially aromatic bicyclic ring containing 1-3 heteroatoms selected from O, S, and N.

In another embodiment, HET is selected from the group consisting of: benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

In another embodiment, HET is selected from the group consisting of: azetidinyl, 1,4-dioxanyl, furanyl, hexahydroazepinyl, isooxazolyl, morpholinyl, oxazolyl, piperazinyl, piperidinyl, pyrrolidinyl, pyridazinyl, pyridyl, pyrimidyl, pyrrolyl, quinazolinyl, quinolyl, quinoxalinyl, thiadiazolyl, thiazolyl, thiophenyl, thiazolyl, and thienyl.

In another embodiment, HET is selected from the group consisting of: pyridazinyl, pyridyl, pyrimidyl, furanyl, thiazolyl, and oxazolyl.

In another embodiment, HET is selected from the group consisting of: pyridazinyl, pyridyl, and pyrimidyl. In yet another embodiment, HET is pyridyl or pyrimidyl. In another embodiment, HET is pyridyl.

In one embodiment, X is —CH═. In another embodiment, X is —N═.

In one embodiment, R¹ is selected from the group consisting of:
(1) phenyl,
(2) naphthyl,
(3) pyridyl, and
(4) pyrimidyl;
wherein each of items (1) to (4) is optionally substituted with one to three groups independently selected from:
   (a) halogen,
   (b) —OR^{a},
   (c) —C(O)—R^{a},
   (d)—C(O)—OR^{a},
   (e)—C(O)NR^{a}R^{b},
   (f) C₁₋₄alkyl, optionally substituted with one to three groups selected from halogen, hydroxy, and —C(O)NR^{a}R^{b},
   (g) C₃₋₆cycloalkyl,
   (h)—SO₂R^{a}, and
   (i) nitrile;

In another embodiment, R¹ is selected from the group consisting of:
(1) phenyl, and
(2) pyridyl;
wherein each of items (1) and (2) is optionally substituted with one to three groups independently selected from:
   (a) halogen,
   (b) —OR^{a},
   (c)—C(O)—R^{a},
   (d) —C(O)—OR^{a},
   (e)—C(O)NR^{a}R^{b},
   (f) C₁₋₄alkyl, optionally substituted with one to three groups selected from halogen, hydroxy, and —C(O)NR^{a}R^{b},
   (g) C₃₋₆cycloalkyl,
   (h)—SO₂R^{a}, and
   (i) nitrile.

In another embodiment, R¹ is selected from the group consisting of:
(1) phenyl, and
(2) pyridyl;
wherein each of items (1) and (2) is optionally substituted with one to three groups independently selected from:
   (a) halogen,
   (b)—OR^{a},
   (c)—C(O)—R^{a},
   (d) —C(O)—OR^{a},
   (e)—C(O)NR^{a}R^{b},
   (f) C₁₋₄alkyl, optionally substituted with one to three groups selected from halogen, hydroxy, and —C(O)NH₂,
   (g) cyclopropyl,
   (h)—SO₂R^{a}, and
   (i) nitrile.

In one embodiment, R² is selected from the group consisting of:
(1) hydrogen,
(2) C₁₋₄alkyl, and
(3) hydroxy.

In another embodiment, R² is hydrogen, methyl or hydroxy. In another embodiment, R² is hydrogen or hydroxy. In yet another embodiment, R² is hydrogen.

In one embodiment, R³ is selected from the group consisting of
(1) C₁₋₄alkyl, optionally substituted with one to three halogens, and
(2) C₃₋₆cycloalkyl.

In another embodiment, R³ is selected from the group consisting of:
(1) methyl,
(2) ethyl,
(3) —CF₃,
(4) —CH₂CF₃, and
(5) cyclopropyl.

In another embodiment, R³ is selected from the group consisting of:
(1) ethyl,
(2) —CF₃,
(3) —CH₂CF₃, and
(4) cyclopropyl.

In yet another embodiment, R³ is ethyl or cyclopropyl.

In one embodiment, each of R⁴ and R⁵ is independently selected from the group consisting of:
(1) hydrogen,
(2) C₁₋₄alkyl, and
(3) halogen.

In another embodiment, each of R⁴ and R⁵ is independently selected from the group consisting of:
(1) hydrogen, and
(2) halogen.

In another embodiment, each of R⁴ and R⁵ is independently selected from the group consisting of:
(1) hydrogen,
(2) chloro,
(3) fluoro, and
(4) bromo.

In another embodiment, R⁴ is fluoro and R⁵ is hydrogen.

In another embodiment, R⁴ is hydrogen and R⁵ is fluoro.

In another embodiment, R⁴ is hydrogen and R⁵ is hydrogen.

In one embodiment, each of R^{a} and R^{b} is independently selected from the group consisting of
(1) hydrogen,
(2) C₁₋₄alkyl, optionally substituted with one to three groups selected from halogen, hydroxy, C₃₋₆cycloalkyl, and —C(O)NH₂, and
(3) C₃₋₆cycloalkyl, optionally substituted with one to three groups selected from halogen, C₁₋₄alkyl, and C₁₋₄alkoxy.

In another embodiment, each of R^{a} and R^{b} is independently selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from halogen, methyl, ethyl, propyl, methoxy, and ethoxy.

In another embodiment, each of R^{a} and R^{b} is independently selected from the group consisting of:
(1) hydrogen,
(2) methyl, optionally substituted with one to three groups selected from halogen, hydroxy, cyclopropyl, and —C(O)NH₂,
(3) ethyl, optionally substituted with one to three groups selected from halogen, hydroxy, cyclopropyl, and —C(O)NH₂, and
(4) cyclopropyl, optionally substituted with one to three groups selected from halogen, methyl, ethyl, propyl, methoxy, and ethoxy.

In yet another embodiment, R^{a} is selected from the group consisting of:
(1) methyl, optionally substituted with one to three groups selected from halogen, hydroxy, cyclopropyl, and —C(O)NH₂,
(2) ethyl, optionally substituted with one to three groups selected from halogen, hydroxy, cyclopropyl, and —C(O)NH₂, and
(3) cyclopropyl, optionally substituted with one to three groups selected from halogen, methyl, ethyl, propyl, methoxy, and ethoxy; and
R^{b} is hydrogen.

In still another embodiment, R^{a} is hydrogen and R^{b} is hydrogen.

In one embodiment, compounds disclosed herein have Formula Ia:

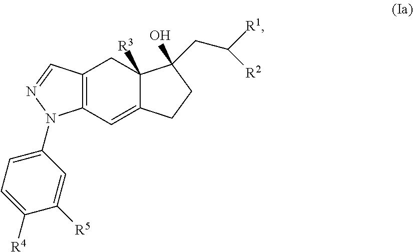

wherein
R¹ is selected from the group consisting of:
(1) phenyl, and
(2) pyridyl;
wherein each of items (1) and (2) is optionally substituted with one to three groups independently selected from:
   (a) halogen,
   (b) —OR^{a},
   (c) —C(O)—R^{a},
   (d) —C(O)—OR^{a},
   (e) —C(O)NR^{a}R^{b},
   (f) C₁₋₆alkyl, optionally substituted with one to three groups selected from halogen, hydroxy, and —C(O)NR^{a}R^{b},
   (g) C₃₋₆cycloalkyl,
   (h) —SO₂R^{a}, and
   (i) nitrile;
      R² is hydrogen or hydroxy;
      R³ is selected from the group consisting of:
(1) ethyl, and
(2) cyclopropyl;
   each of R⁴ and R⁵ is independently selected from the group consisting of:
(1) hydrogen, and
(2) halogen; and
   each of R^{a} and R^{b} is independently selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from halogen, methyl, ethyl, propyl, methoxy, and ethoxy.

In another embodiment, compounds disclosed herein have Formula Ib:

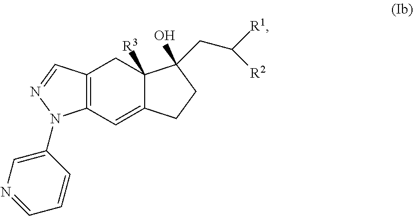

wherein
R¹ is selected from the group consisting of:
(1) phenyl, and
(2) pyridyl;
wherein each of items (1) and (2) is optionally substituted with one to three groups independently selected from:
   (a) halogen,
   (b) —OR^{a},
   (c) —C(O)—R^{a},
   (d) —C(O)—OR^{a},
   (e) —C(O)NR^{a}R^{b},
   (f) C₁₋₆alkyl, optionally substituted with one to three groups selected from halogen, hydroxy, and —C(O)NR^{a}R^{b},
   (g) C₃₋₆cycloalkyl,
   (h) —SO₂R^{a}, and
   (i) nitrile;
      R² is hydrogen or hydroxy;
      R³ is selected from the group consisting of
(1) ethyl, and
(2) cyclopropyl; and
   each of R^{a} and R^{b} is independently selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from halogen, methyl, ethyl, propyl, methoxy, and ethoxy.

In one embodiment of Formula Ia or Ib, R¹ is selected from the group consisting of
(1) phenyl, and
(2) pyridyl;
wherein each of items (1) and (2) is optionally substituted with one to three groups independently selected from:
   (a) halogen,
   (b)—OR^{a},
   (c) —C(O)—R^{a},
   (d) —C(O)—OR^{a},
   (e) —C(O)NH₂,
   (f) C₁₋₄alkyl, optionally substituted with one to three groups selected from halogen, hydroxy, and —C(O)NH₂,
   (g) cyclopropyl,
   (h) —SO₂R^{a}, and
   (i) nitrile; and
      R^{a} is selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from methyl, ethyl, propyl, and methoxy

In another embodiment of compounds of Formula Ia or Ib, R¹ is selected from the group consisting of:
(1) phenyl, and
(2) pyridyl;
wherein each of items (1) and (2) is optionally substituted with one to three groups independently selected from:
   (a) hydrogen,
   (b) halogen,
   (c) C₁₋₄alkyl, optionally substituted with one to three halogen atoms,
   (d) C₃₋₆cycloalkyl,
   (e) C₁₋₄alkoxy, and
   (f) nitrile.

In another embodiment of compounds of Formula Ia or Ib, R¹ is selected from the group consisting of:
(1) phenyl, and
(2) pyridyl;
wherein each of items (1) and (2) is optionally substituted with one to three groups independently selected from:
   (a) hydrogen,
   (b) halogen,
   (c) C₁₋₄alkyl, optionally substituted with one to three halogen atoms,
   (d) C₁₋₄alkoxy, and
   (e) nitrile;

In yet another embodiment of compounds of Formula Ia or Ib, R¹ is selected from the group consisting of:
(1) phenyl, and
(2) pyridyl;
wherein each of items (1) and (2) is optionally substituted with one to three groups independently selected from:
   (a) hydrogen,
   (b) fluoro,
   (c) chloro,
   (d) bromo,
   (e) methyl,
   (f) ethyl,
   (g) propyl,
   (h) trifluoromethyl,
   (i) cyclopropyl,
   (j) methoxy,
   (k) ethoxy, and
   (l) nitrile.

In one embodiment of Formula Ia or Ib, R² is hydrogen or hydroxy. In another embodiment, R² is hydrogen. In yet another embodiment, R² is hydroxy.

In one embodiment of Formula Ia, R⁴ is hydrogen or fluoro, and R⁵ is hydrogen or fluoro.

In another embodiment of Formula Ia, R⁴ is hydrogen and R⁵ is hydrogen.

The present invention also encompasses a pharmaceutical composition comprising a compound disclosed herein in combination with a pharmaceutically acceptable carrier.

The present invention further encompasses a method for treating a glucocorticoid receptor mediated disease or condition in a mammalian patient in need thereof. In one embodiment, the method comprises administering to the patient a compound disclosed herein in an amount that is effective for treating the glucocorticoid receptor mediated disease or condition. It has surprising been found that compounds disclosed herein possess superior properties as compared to known compounds. For example, the instant compounds provide good potencies in GITAR and improved selectivity as evidenced by good Emax values in GITAR assays.

In one embodiment of the method described above, the glucocorticoid receptor mediated disease or condition is selected from the group consisting of tissue rejection, leukemias, lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity, metabolic syndrome, inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, cirrhosis, inflammatory scalp alopecia, panniculitis, psoriasis, discoid lupus erythematosus, inflamed cysts, atopic dermatitis, pyoderma gangrenosum, pemphigus vulgaris, buflous pemphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma, Human Immunodeficiency Virus (HIV), cell apoptosis, cancer, Kaposi's sarcoma, retinitis pigmentosa, cognitive performance, memory and learning enhancement, depression, addiction, mood disorders, chronic fatigue syndrome, schizophrenia, sleep disorders, and anxiety.

Specifically, compounds disclosed herein may be used for treating or preventing the following exemplary, non-limiting diseases or conditions.

### 1. Allergic States

For the control of severe or incapacitating allergic conditions not responsive to adequate trials of conventional treatment; seasonal or perennial allergic rhinitis; bronchial asthma; contact dermatitis; atopic dermatitis; serum sickness; and drug hypersensitivity reactions.

### 2. Rheumatic Disorders

For short-term administration during an acute episode or exacerbation of: psoriatic arthritis; rheumatoid arthritis including juvenile rheumatoid arthritis; ankylosing spondylitis; acute and subacute bursitis; acute nonspecific tenosynovitis; acute gouty arthritis; post-traumatic osteoarthritis; synovitis of osteoarthritis; and epicondylitis

### 3. Dermatologic Diseases

For treating or preventing pemphigus; bullous dermatitis herpetiformis; severe erythema multiforme (Stevens-Johnson syndrome); exfoliative dermatitis; mycosis fungo'ides; severe psoriasis; and severe seborrheic dermatitis.

### 4. Ophthalmic Diseases

For treating or preventing severe acute and chronic allergic and inflammatory processes involving the eye and its adnexa such as: allergic conjunctivitis; keratitis; allergic corneal marginal ulcers; herpes zoster ophthalmicus; iritis and iridocyclitis; chorioretinitis; anterior segment inflammation; diffuse posterior uveitis and choroiditis; optic neuritis; and sympathetic ophthalmia.

### 5. Endocrine Disorders

For treating or preventing primary or secondary adrenocortical insufficiency; congenital adrenal hyperplasia; nonsuppurative thyroiditis; and hypercalcemia associated with cancer.

### 6. Respiratory Diseases

For treating or preventing symptomatic sarcoidosis; Loffler's syndrome not manageable by other means; berylliosis; fulminating or disseminated pulmonary tuberculosis when concurrently accompanied by appropriate antituberculous chemotherapy; and aspiration pneumonitis.

### 7. Hematologic Disorders

For treating or preventing idiopathic thrombocytopenic purpura in adults; secondary thrombocytopenia in adults; acquired (autoimmune) hemolytic anemia; erythroblastopenia WC anemia); and congenital (erythroid) hypoplastic anemia.

### 8. Neoplastic Diseases

For palliative management of leukemias and lymphomas in adults; and acute leukemia of childhood. For the treatment of diverse neoplastic diseases such as brain cancer, bone cancer, basal cell carcinoma, adenocarcinoma, lip cancer, mouth cancer, esophogeal cancer, small bowel cancer, stomach cancer, colon cancer, rectal cancer, liver cancer, bladder cancer, pancreas cancer, ovary cancer, cervical cancer, lung cancer, breast cancer, head and neck cancer, skin cancer, prostate cancer, gall bladder cancer, thyroid cancer and renal cell carcinoma.

### 9. Edematous States

For inducing a diuresis or remission of proteinuria in the nephrotic syndrome without uremia, of the idiopathic type or that due to lupus erythematosus. Compounds of the invention may be used to treat patients with cerebral edema from various causes. It may be used also in the preoperative preparation of patients with increased intracranial pressure secondary to brain tumors, and also for palliation of patients with inoperable or recurrent brain neoplasms, and in the management of cerebral edema associated with neurosurgery. Some patients with cerebral edema due to head injury or pseudotumor cerebri also may benefit from therapy with compounds of the invention.

### 10. Gastrointestinal Diseases

For treating or preventing ulcerative colitis and regional enteritis.

### 11. Miscellaneous

For treating tuberculous meningitis with subarachnoid block or impending block when concurrently accompanied by appropriate antituberculous chemotherapy; trichinosis with neurologic or myocardial involvement; during an exacerbation or as maintenance therapy in selected cases of: systemic lupus erythematosus and acute rheumatic carditis; in combination with ondansetron for the management of nausea and vomiting associated with cisplatin and non-cisplatin emetogenic chemotherapy. The compounds of the invention are also useful for treating or preventing hypertension, vascular inflammation, urinary incontinence and multiple sclerosis.

### 12. CNS Diseases

For the treatment of HPA axis dysregulation in psychiatric disease, schizophrenia, bipolar disorder, psychotic major depression and posttraumatic syndrome.

Another embodiment of the invention encompasses a method of selectively modulating the activation, repression, agonism and antagonism effects of the glucocorticoid receptor in a mammal. In one embodiment, the method comprises administering to the mammal a compound disclosed herein in an amount that is effective to modulate the glucocorticoid receptor.

The invention is described using the following definitions unless otherwise indicated.

The term “halogen” or “halo” includes F, Cl, Br, and I.

The term “alkyl” means linear or branched structures and combinations thereof, having the indicated number of carbon atoms. For example, C₁₋₆alkyl includes, but is not limited to, methyl, ethyl, propyl, 2-propyl, s- and t-butyl, butyl, pentyl, hexyl, and 1,1-dimethylethyl.

The term “cycloalkyl” means mono-, bi- or tri-cyclic structures, optionally combined with linear or branched structures, having the indicated number of carbon atoms. Non-limiting examples of C₃₋₆cycloalkyl include cyclopropyl, cyclobutyl, cyclopentyl and cyclohexyl.

The term “alkoxy” means alkoxy groups of a straight, branched or cyclic configuration having the indicated number of carbon atoms. For example, C₁₋₄alkoxy includes, but is not limited to, methoxy, ethoxy, propoxy, isopropoxy, butoxy.

The term “optionally substituted” means “unsubstituted or substituted,” and therefore, the generic structural formulas described herein encompass compounds containing the specified optional substituent as well as compounds that do not contain the optional substituent. Each variable is independently defined each time it occurs within the generic structural formula definitions.

For all of the above definitions, each reference to a group is independent of all other references to the same group when referred to in the Specification. For example, if both R₁ and R₂ are C₁₋₄alkyl groups, the definitions of C₁₋₄alkyl are independent of each other and R₁ and R₂ may be different C₁₋₄alkyl groups, for example, methyl and ethyl.

The term “treating” encompasses not only treating a patient to relieve the patient of the signs and symptoms of the disease or condition but also prophylactically treating an asymptomatic patient to prevent the onset of the disease or condition or preventing, slowing or reversing the progression of the disease or condition.

The term “amount effective for treating” is intended to mean that amount of a compound that will elicit the biological or medical response of a tissue, a system, animal or human that is being sought.

### Optical Isomers-Diastereomers-Geometric Isomers-Tautomers

Compounds described herein may contain an asymmetric center and may thus exist as enantiomers. Where the compounds according to the invention possess two or more asymmetric centers, they may additionally exist as diastereomers. When bonds to the chiral carbon are depicted as straight lines in the formulas of the invention, it is understood that both the (R) and (S) configurations of the chiral carbon, and hence both enantiomers and mixtures thereof, are embraced within the formulas. The present invention includes all such possible stereoisomers as substantially pure resolved enantiomers, racemic mixtures thereof, as well as mixtures of diastereomers. The present invention includes all stereoisomers of the compounds disclosed herein and pharmaceutically acceptable salts thereof.

Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound disclosed herein may be obtained by stereospecific synthesis using optically pure starting materials or reagents of known configuration.

When compounds described herein contain olefinic double bonds, unless specified otherwise, such double bonds are meant to include both E and Z geometric isomers.

Some of the compounds described herein may exist with different points of attachment of hydrogen, referred to as tautomers. For example, compounds including carbonyl —CH2C(O)— groups (keto forms) may undergo tautomerism to form hydroxyl —CH—C(OH)— groups (enol forms). Both keto and enol forms, individually as well as mixtures thereof, are included within the scope of the present invention.

### Isotopes

In the compounds disclosed herein, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds disclosed herein. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds disclosed herein can be prepared without undue experimentation by conventional techniques well known to those skilled in the art or by processes analogous to those described in the Schemes and Examples herein using appropriate isotopically-enriched reagents and/or intermediates.

### Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include, for example, aluminum, ammonium, calcium, copper (ic and ous), ferric, ferrous, lithium, magnesium, manganese (ic and ous), potassium, sodium, and zinc salts.

Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, and tromethamine.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, maleic, malic, mandelic, methanesulfonic, mucic, nitric, pamoic, pantothenic, phosphoric, succinic, sulfuric, tartaric, and p-toluenesulfonic acid.

### Solvates

The present invention includes within its scope solvates of the compounds disclosed herein. As used herein, the term “solvate” refers to a complex of variable stoichiometry formed by a solute (i.e., a compound of Formula I or Ia) or a pharmaceutically acceptable salt thereof and a solvent that does not interfere with the biological activity of the solute. Examples of solvents include, but are not limited to water, ethanol, and acetic acid. When the solvent is water, the solvate is known as hydrate. Hydrates include, but are not limited to, hemi-, mono, sesqui-, di- and trihydrates.

### Prodrugs

The present invention includes within its scope the prodrugs of the compounds disclosed herein. In general, such prodrugs will be functional derivatives of the compounds of this invention which are readily convertible in vivo into the compound disclosed herein. Thus, in the methods of treatment of the present invention, the term “administering” shall encompass the treatment of the various conditions described with a compound of Formula I, Ia, or Ib, or with a compound which may not be a compound of Formula I, Ia, or Ib, but which converts to a compound of Formula I, Ia, or Ib in vivo after administration to the patient. Conventional procedures for the selection and preparation of suitable prodrug derivatives are described, for example, in “Design of Prodrugs,” ed. H. Bundgaard, Elsevier, 1985.

### Combination Therapy

Disclosed herein also includes a method for treating a glucocorticoid receptor mediated disease comprising concomitantly administering to a patient in need thereof a compound of the invention and one or more active agents.

For treating or preventing asthma or chronic obstructive pulmonary disease, the compounds of the invention may be combined with one or more agents selected from the group consisting of: S-agonists (e.g., salmeterol), theophylline, anticholinergics (e.g., atropine and ipratropium bromide), cromolyn, nedocromil and leukotriene modifiers (e.g., montelukast).

For treating or preventing inflammation, the compounds of the invention may be combined with one or the following: a salicylate, including acetylsalicylic acid, a non-steroidal anti-inflammatory drug, including indomethacin, sulindac, mefenamic, meclofenamic, tolfenamic, tolmetin, ketorolac, dicofenac, ibuprofen, naproxen, fenoprofen, ketoprofen, flurbiprofen and oxaprozin, a TNF inhibitor, including etanercept and infliximab, an IL-1 receptor antagonist, a cytotoxic or immunosuppressive drug, including methotrexate, leflunomide, azathioprine and cyclosporine, a gold compound, hydroxychloroquine or sulfasalazine, penicillamine, darbufelone, and a p38 kinase inhibitor.

The compounds of the invention may also be used in combination with bisphonates such as alendronate, SERMs (selective estrogen receptor modulators) or cathepsin K inhibitors to treat a glucocorticoid mediated disease and simultaneously causes ostepenia or osteoporosis.

The compounds of the invention may also be used in combination with bone anabolic agents such as PTH, Androgens, SARMs (selective androgen receptor modulators), to treat a glucocorticoid mediated disease and simultaneously induces bone loss as exhibited by osteopenia or osteoporosis.

The compounds of the invention may further be used in combination with active agents used to treat age-related sarcopenia or cachexia to treat a glucocorticoid mediated diseases and simultaneously inhibit muscle loss, sarcopenia and frailty.

The pharmaceutical composition of the present invention comprises a compound disclosed herein or a pharmaceutically acceptable salt thereof as the active ingredient, and may also contain a pharmaceutically acceptable carrier and optionally other therapeutic ingredients.

The magnitude of prophylactic or therapeutic dose of a compound disclosed herein will vary with the nature and the severity of the condition to be treated and with the particular compound and its route of administration. It will also vary according to a variety of factors including the age, weight, general health, sex, diet, time of administration, rate of excretion, drug combination and response of the individual patient. In general, the daily dose ranges from about 0.001 mg to about 100 mg per kg body weight of a mammal, preferably 0.01 mg to about 10 mg per kg. On the other hand, it may be necessary to use dosages outside these limits in some cases.

The amount of active ingredient that may be combined with the carrier materials to produce a single dosage form will vary depending upon the host treated and the particular mode of administration. For example, a formulation intended for oral administration to humans may contain from about 0.5 mg to about 5 g of active agent compounded with an appropriate and convenient amount of carrier material which may vary from about 5 to about 95 percent of the total composition. Dosage unit forms will generally contain from about 1 mg to about 2 g of an active ingredient, typically 25 mg, 50 mg, 100 mg, 200 mg, 300 mg, 400 mg, 500 mg, 600 mg, 800 mg, or 1000 mg.

For the treatment of glucocorticoid receptor mediated diseases the compound disclosed herein may be administered orally, topically, parenterally, by inhalation spray or rectally in dosage unit formulations containing conventional non-toxic pharmaceutically acceptable carriers, adjuvants and vehicles. The term parenteral as used herein includes subcutaneous, intravenous, intramuscular, intrasternal injection or infusion techniques. In addition to the treatment of warm-blooded animals such as mice, rats, horses, cattle, sheep, dogs, cats, etc., the compound of the invention is effective in the treatment of humans.

The pharmaceutical compositions containing the active ingredient may be in a form suitable for oral use, for example, as tablets, troches, lozenges, solutions, aqueous or oily suspensions, dispersible powders or granules, emulsions, hard or soft capsules, syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions contain the active compound in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more colouring agents, one or more flavouring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

The pharmaceutical compositions of the invention may also be in the form of an oil-in-water emulsion. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

For topical use, creams, ointments-, gels, solutions or suspensions, etc., containing a compound disclosed herein are employed. (For purposes of this application, topical application shall include mouth washes and gargles.) Topical formulations may generally be comprised of a pharmaceutical carrier, cosolvent, emulsifier, penetration enhancer, preservative system, and emollient.

The ability of the compounds disclosed herein to selectively modulate glucocorticoid receptors makes them useful for treating, preventing or reversing the progression of a variety of inflammatory and autoimmune diseases and conditions. Thus, the compounds of the present invention are useful to treat, prevent or ameliorate the following diseases or conditions: inflammation, tissue rejection, auto-immunity, various malignancies, such as leukemias and lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity and metabolic syndrome.

The compounds disclosed herein are also useful for treating, preventing or reversing the progression of disease states involving systemic inflammation such as inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, and cirrhosis.

The compounds disclosed herein are useful for treating, preventing or reversing the progression of a variety of topical diseases such as inflammatory scalp alopecia, panniculitis, psoriasis, discoid lupus erythematosus, inflamed cysts, atopic dermatitis, pyoderma gangrenosum, pemphigus vulgaris, buflous pemphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitis, erythema nodosum, acne, hirsutism, toxic epidermal necrolysis, erythema multiform, cutaneous T-cell lymphoma.

The compounds disclosed herein are also useful in treating, preventing or reversing the progression of disease states associated with Human Immunodeficiency Virus (HIV), cell apoptosis, and cancer including, but not limited to, Kaposi's sarcoma, immune system activation and modulation, desensitization of inflammatory responses, IIL-1 expression, natural killer cell development, lymphocytic leukemia, and treatment of retinitis pigmentosa. Cognitive and behavioral processes are also susceptible to glucocorticoid therapy where antagonists would potentially be useful in the treatment of processes such as cognitive performance, memory and learning enhancement, depression, addiction, mood disorders, chronic fatigue syndrome, schizophrenia, stroke, sleep disorders, and anxiety.

The invention also encompasses a method for treating a glucocorticoid receptor mediated disease comprising concomitantly administering to a patient in need of such treatment a compound disclosed herein and one or additional more agents. For treating or preventing asthma or chronic obstructive pulmonary disease, the compounds disclosed herein may be combined with one or more agents selected from the group consisting of Θ-agonists (e.g., salmeterol), theophylline, anticholinergics (e.g., atropine and ipratropium bromide), cromolyn, nedocromil and leukotriene modifiers (e.g., montelukast). For treating or preventing inflammation, the compounds disclosed herein may be combined with one or the following: a salicylate, including acetylsalicylic acid, a non-steroidal antiinflammatory drug, including indomethacin, sulindac, mefenamic, meclofenamic, tolfenamic, tolmetin, ketorolac, dicofenac, ibuprofen, naproxen, fenoprofen, ketoprofen, flurbiprofin and oxaprozin, a TNF inhibitor, including etanercept and infliximab, an IL-1 receptor antagonist, a cytotoxic or immunosuppressive drug, including methotrexate, leflunomide, azathioprine and cyclosporine, a gold compound, hydroxychloroquine or sulfasalazine, penicillamine, darbufelone, and a p38 kinase inhibitor. The compound disclosed herein may also be used in combination with bisphonates such as alendronate to treat a glucocorticoid mediated disease and simultaneously inhibit osteoclast-mediated bone resorption.

Unless noted otherwise, the following abbreviations have the indicated meanings:

DMF=N,N-dimethylformamide

DMSO=dimethyl sulfoxide

e.q.=equivalents)

EtOAc=ethyl acetate

g=gram(s)

h=hour(s)

L=liter(s)

mg=milligram(s)

min=minute(s)

mL=milliliter(s)

mol.=mole(s)

mmol.=millimole(s)

MW=molecular weight

Ph=phenyl

1,2-Ph=1,2-benzenediyl

r.t.=room temperature

sat.=saturated

THF=tetrahydrofuran

v=volume

w=weight

### Alkyl Group Abbreviations

Me=methyl

Et=ethyl

n-Pr=nonnal propyl

i-Pr=isopropyl

n-Bu=nonnal butyl

i-Bu=isobutyl

s-Bu=secondary butyl

t-Bu=tertiary butyl

^{c}Pr=cyclopropyl

^{c}Bu=cyclobutyl

### METHODS OF SYNTHESIS

Generally, compounds of the present invention may be synthesized by using the following synthetic schemes and examples:

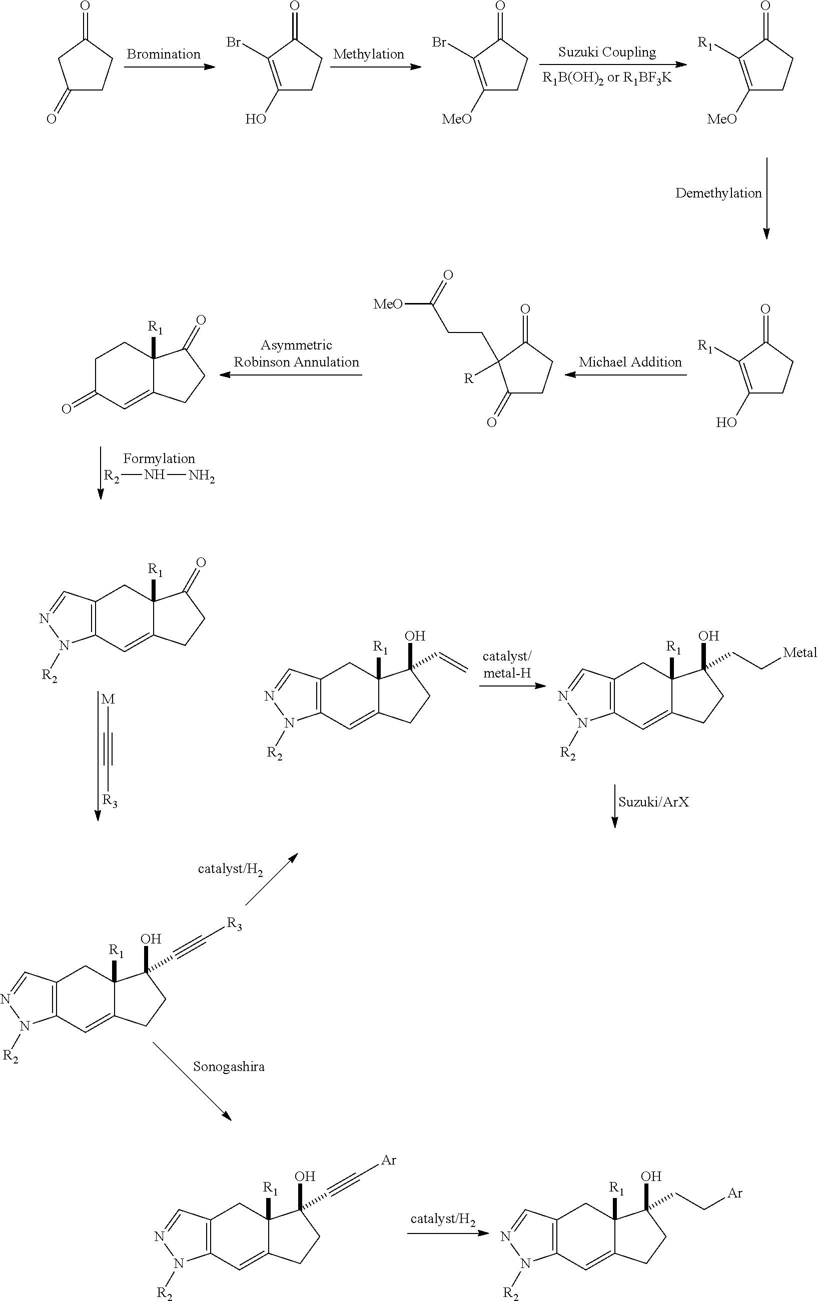

### Intermediate 1 (i-1)

### 2-Cyclopropyl-3-hydroxycyclopent-2-en-1-one (i-1)

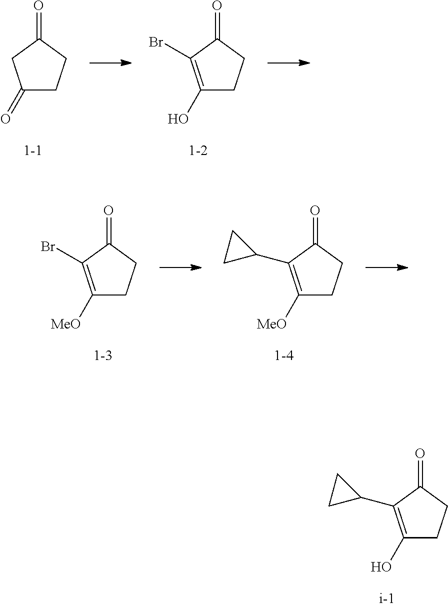

### Step A: 2-Bromo-3-hydroxycyclopent-2-en-1-one (1-2)

48% w/w aqueous HBr (28.8 ml, 255 mmol), and potassium bromate (14.04 g, 84 mmol) in water (127 ml) were added in turn to a suspension of cyclopentane-1,3-dione 1-1 (25 g, 255 mmol) in water (127 ml). The reaction was stirred for 15 min at ambient temperature to afford a white suspension. The desired product was isolated by filtration, affording 1-2 (37.2 g, 82%) as a white solid. MS (ESI): m/z=178.97 (MH⁺).

### Step B: 2-Bromo-3-methoxycyclopent-2-en-1-one (1-3)

2-Bromo-3-hydroxycyclopent-2-en-1-one 1-2 (22.7 g, 128 mmol), trimethyl orthoformate (15.59 ml, 141 mmol), and Amberlite® IR-120 Plus (6.80 g, 6797 mmol) (prewashed with MeOH) in MeOH (650 ml) were heated at 70° C. overnight. The reaction mixture was filtered off and the solvent was evaporated under reduced pressure to afford 1-3 (24.1 g, 98%) as pale yellow solid. MS (ESI): m/z=191.01 (MH⁺).

### Step C: 2-Cyclopropyl-3-methoxycyclopent-2-en-1-one (1-4)

2-Bromo-3-methoxycyclopent-2-en-1-one 1-3 (3.55 g, 18.58 mmol), potassium cycloproyltrifluoroborate (2.89 g, 19.51 mmol), 1,1′-bis(di-tert-butylphosphino)ferrocene palladium dichloride (0.363 g, 0.558 mmol), and Cs₂CO₃ (18.17 g, 55.8 mmol) in Toluene (45.1 ml)/Water (4.51 ml) (degassed with N₂ bubbling) were heated at 100° C. for 2 h. The reaction was diluted with ethyl acetate and dried (MgSO₄), then filtered through a pad of florisil, rinsing with ethyl acetate and the solvent was evaporated under reduced pressure. Purification by Kugelohr distillation (150° C. @ 5 Torr) afforded 1-4 (2.33 g, 82%) as a light yellow oil. MS (ESI): m/z=153.10 (MH⁺).

### Step D: 2-Cyclopropyl-3-hydroxycyclopent-2-en-1-one (i-1)

2-Cyclopropyl-3-methoxycyclopent-2-en-1-one 1-4 (29.0 g, 191 mmol) was dissolved in MeOH (382 ml) and 6M HCl (3 ml) was added. The reaction was stirred at ambient temperature for 3 h, then concentrated and azeotroped with THF (100 ml) to afford i-1 (26.0 g, 100%) as a yellow solid. MS (ESI): m/z=154.1 (M+16).

### Intermediate 2 (i-2)

### (3R,3aR)-3a-Cyclopropyl-3-ethynyl-3-hydroxy-6-oxo-2,3,3a,4,5,6-hexahydro-1H-indene-5-carbaldehyde

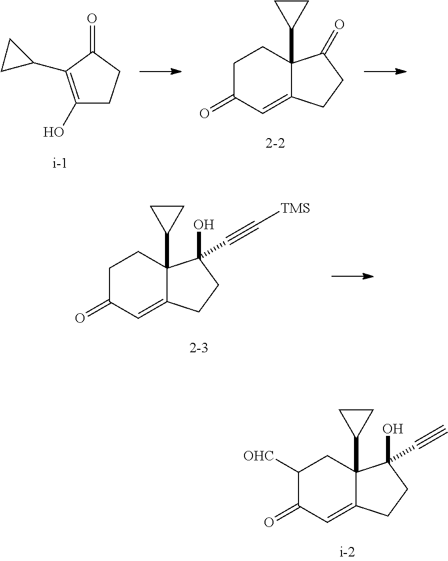

### Step A: (7aR)-7a-Cyclopropyl-2,3,7,7a-tetrahydro-1H-indene-1,5(6H)-dione (2-2)

2-Cyclopropyl-3-hydroxycyclopent-2-en-1-one i-1 (27.0 g, 195 mmol), methyl vinyl ketone (41.1 g, 586 mmol), L-proline (24.75 g, 215 mmol) and AcOH (24.17 g, 391 mmol) in water (390 ml) were heated at 75° C. for 48 h. The reaction mixture was allowed to cool to ambient temperature. Slowly added solid K₂CO₃ until basic and then extracted with CH₂Cl₂. The organic portion was dried (MgSO₄) and the solvent was evaporated under reduced pressure. The triketone was dissolved in DMF (100 ml). L-proline was grounded with a mortar and pestle and suspended in DMF (160 ml) and stirred for 1 h. The triketone solution was added and the dark solution was stirred for 48 h at ambient temperature. The solution was heated to 70° C. and then added H₂SO₄ (6 ml). The solution was heated to 95° C. for 1 h and then allowed to cool to ambient temperature. The solvent was removed under reduced pressure. The residue was dissolved in 300 ml CH₂Cl₂ and then was washed with 1:1 saturated NaHCO₃/brine (150 ml). The organic portion was dried (MgSO₄) and the solvent was removed under reduced pressure. The residue was purified by column chromatography on silica gel eluting with hexanes to EtOAc to give 2-2 (18 g, 73%) as a yellow oil. MS (ESI): m/z=191.10 (MH⁺).

### Step B: (1S,7aR)-7a-Cyclopropyl-1-hydroxy-1-[trimethylsilyl)ethynyl]-1,2,3,6,7,7a-hexahydro-5H-inden-5-one (2-3)

A 2.5 M solution of N-Butyllithium (66.2 ml, 166 mmol) in hexanes was added to a solution of trimethylsilylacetylene (23.56 ml, 170 mmol) in THF (252 ml) at −78° C. and the resulting solution was stirred at this temperature for 30 min. A solution of 2-2 (18.00 g, 95 mmol) in THF (50 ml) was added and the resulting solution stirred for 30 min at −78° C. The reaction was quenched with KH₂PO₄ (saturated) and the mixture was extracted with ethyl acetate. The organic portion was washed with brine, dried (MgSO₄) and the solvent removed under reduced pressure. The residue was purified by column chromatography on silica gel eluting with hexanes to EtOAc to give 2-3 (23.5 g, 86%) as a yellow solid. MS (ESI): m/z 271.10 (M-18).

### Step C: (3R,3aR)-3a-Cyclopropyl-3-ethynyl-3-hydroxy-6-oxo-2,3,3a,4,5,6-hexahydro-1H-indene-5-carbaldehyde (i-2)

2.0 M Lithium diisopropylamide (217 ml, 434 mmol) is added dropwise to a solution of 2-3 in THF (693 ml) over 40 min and the thick, brown mixture is stirred for 1 h. Methyl formate (52 g, 867 mmol), dissolved in 100 ml THF, is added and the mixture is stirred for an additional 2 h. The reaction was quenched with 1 N HCl until acidic followed by the removal of the cooling bath. After reaching ambient temperature, the mixture is extracted with EtOAc. The organic portion was washed with brine, dried (MgSO₄) and the solvent was evaporated under reduced pressure. The residue was dissolved in MeOH (427 ml) and then added K₂CO₃ (23.58 g, 171 mmol). The mixture was stirred for 3 h. The solvent was evaporated under reduced pressure. The mixture was acidified with 1N HCl and then extracted with EtOAc. The organic portion was washed with brine, dried (MgSO₄) and the solvent was removed under reduced pressure to provide i-2 (20.8 g, 100%) as an orange oil. MS (ESI): m/z 245.10 (MH⁺).

### Intermediate 3 (i-3)

### (3R,3aS)-3a-Ethyl-3-ethynyl-3-hydroxy-6-oxo-2,3,3a,4,5,6-hexahydro-1H-indene-5-carbaldehyde

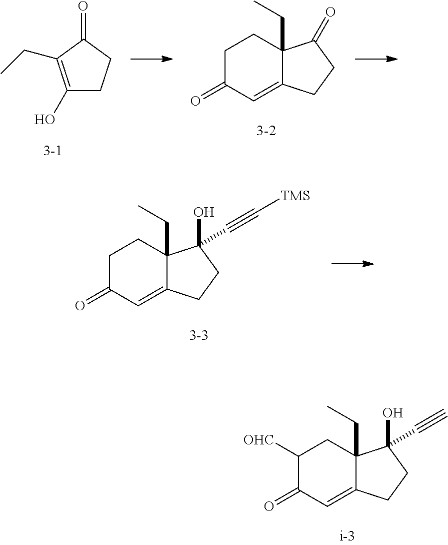

### Step A: (7aS)-7a-Ethyl-2,3,7,7a-tetrahydro-1H-indene-1,5(6H)-dione (3-2)

2-Ethylpentane-1,3-dione 3-1 (25.0 g, 198 mmol), methyl vinyl ketone (23.6 g, 337 mmol), and AcOH (0.714 g, 11.9 mmol) in water (50 ml) were heated at 70° C. for 4 h. Allowed to cool to ambient temperature and then stirred overnight. The mixture was extracted with CH₂Cl₂. The organic portion was washed with brine, dried over MgSO4 and the solvent evaporated under reduced pressure. The triketone was dissolved in DMF (25 ml). L-proline (2.35 g, 20.38 mmol) was grounded with a mortar and pestle and suspended in DMF (100 ml) and stirred for 1 h. The triketone solution was added and the dark solution was stirred for 48 h. The solution was heated to 70° C. In a 200 ml RBF is added DMF (30 ml) and then cooled to −20° C. Added H₂SO₄ (1.6 ml) dropwise maintaining the temperature between −15 and −20° C. Added 20 ml of this solution to the reaction mixture. Heated to 95° C. for 1 h. Added the remaining H₂SO₄/DMF solution and continued to heat at 95° C. for 3 h. The mixture was allowed to cool and then stirred overnight. The solution was concentrated. Dissolved in 1 L of CH₂Cl₂ and then washed with 500 ml of 1:1 brine/sat NaCO₃. The organic portion was dried (MgSO₄) and the solvent was evaporated under reduced pressure. The residue was purified by column chromatography on silica gel eluting with hexane to EtOAc to give 3-1 (21 g, 57.8%) as an orange oil. MS (ESI): m/z=179.08 (MH⁺).

### Step B: (1S,7aS)-7a-Ethyl-1-hydroxy-1-[(trimethylsilyl)ethynyl]-1,2,3,6,7,7a-hexahydro-5H-inden-5-one (3-3)

A 2.5 M solution of N-Butyllithium (39.3 ml, 98.6 mmol) in hexanes was added to a solution of trimethylsilylacetylene (9.64 g, 170 mmol) in THF (100 ml) at −78° C. and the resulting solution was stirred at this temperature for 30 min. A solution of 3-2 (10.00 g, 56.1 mmol) in THF (25 ml) was added and the resulting solution stirred for 30 min at −78° C. The reaction was quenched with KH₂PO₄ (saturated) and the mixture was extracted with ethyl acetate. The organic portion was washed with brine, dried (MgSO₄) and the solvent removed under reduced pressure. The residue was purified by column chromatography on silica gel eluting with hexanes to 55% EtOAc/hexanes to give 3-3 (13 g, 84%) as an orange oil. MS (ESI): m/z=277.03 (MH⁺).

### Step C: (3R,3aS)-3a-Ethyl-3-ethynyl-3-hydroxy-6-oxo-2,3,3a,4,5,6-hexahydro-1H-indene-5-carbaldehyde (i-3)

2.0 M Lithium diisopropylamide (18.1 ml, 36.2 mmol) is added dropwise to a solution of 3-3 in 1:1 THF/Et₂O (50 ml) at −78° C. over 20 min. An additional 25 ml THF was added and the thick, brown mixture is stirred for 1 h at −78° C. Methyl formate (4.34 g, 72.3 mmol) is added and the mixture is stirred for an additional 3 h at −78° C. The reaction was quenched with 1 N HCl until acidic followed by the removal of the cooling bath. After reaching ambient temperature, the mixture is extracted with EtOAc. The organic portion was washed with brine, dried (MgSO₄) and the solvent was evaporated under reduced pressure. The residue was dissolved in MeOH (50 ml) and then added K₂CO₃ (2.2 g, 16.1 mmol). The mixture was stirred for 3 h. The solvent was evaporated under reduced pressure. The mixture was acidified with 1 N HCl and then extracted with EtOAc. The organic portion was washed with brine, dried (MgSO₄) and the solvent was removed under reduced pressure to provide i-3 (1.87 g, 100%) as an orange oil. MS (ESI): m/z=233.14 (MH⁺).

### Intermediates 4-11 (i-4-i-11)

### General Procedure for the Synthesis of Intermediates i-4 to i-11

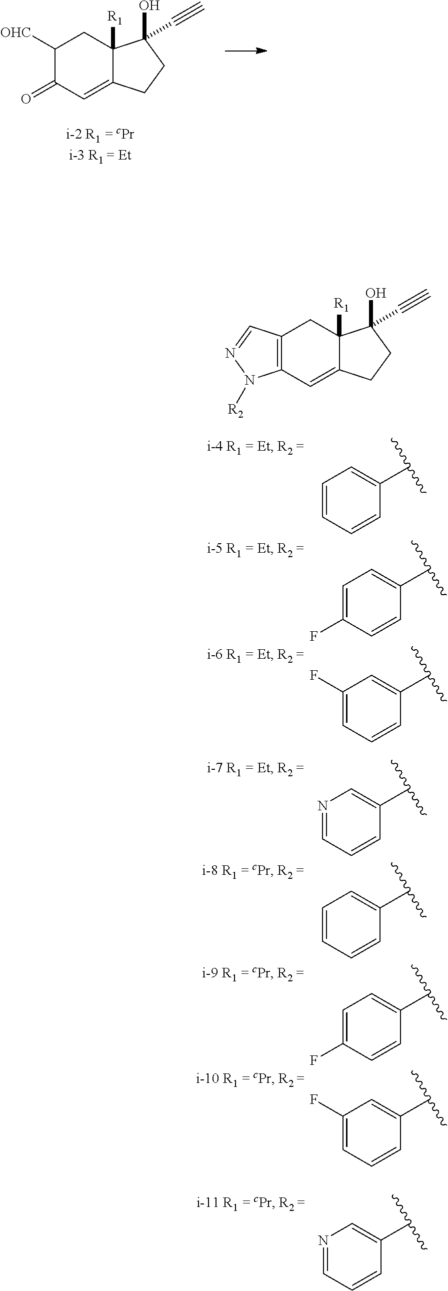

### (4aS,5R)-4a-Ethyl-5-ethynyl-1-phenyl-1,4,4a,5,6,7-hexahydrocyclopenta[f]indazol-5-ol (i-4)

To a stirred solution of i-3 (3.0 g, 12.92 mmol) in AcOH (50 ml) was added phenylhydrazine hydrochloride (2.24 g, 15.5 mmol) and sodium acetate (1.27 g, 15.5 mmol). The mixture was stirred for 4 h. The mixture was slowly poured into a cold saturated NaHCO₃ solution. The mixture was extracted with EtOAc. The organic portion was washed with saturated NaHCO3, brine, dried (MgSO₄) and the solvent was evaporated under reduced pressure. The residue was purified by column chromatography on silica gel eluting with hexane to EtOAc to give i-4 (3.05 g, 78%) as an orange foam. MS (ESI): m/z=305.16 (MH⁺).

Similarly, i-5-i-11 can be prepared from the appropriate starting materials using the procedure similar to that for i-4:
Intermediate i-5—(4aS,5R)-4a-Ethyl-5-ethynyl-1-(4-fluorophenyl)1-1,4,4a,5,6,7-hexahydrocyclopenta[f]indazol-5-ol (i-5) MS (ESI): m/z=323.00 (MH⁺).
Intermediate i-6—(4aS,5R)-4a-Ethyl-5-ethynyl-1-(3-fluorophenyl)1-1,4,4a,5,6,7-hexahydrocyclopenta[f]indazol-5-ol 0-6) MS (EST): m/z=323.11 (MH⁺).
Intermediate i-7—(4aS,5R)-4a-Ethyl-5-ethynyl-1-(pyridin-3-yl)-1,4,4a,5,6,7-hexahydrocyclopenta[f]indazol-5-ol (i-7) MS (ESI): m/z=306.12 (MH⁺).
Intermediate i-8—(4aR,5R)-4a-Cyclopropyl-5-ethynyl-1-phenyl-1,4,4a,5,6,7-hexahydrocyclopenta[f]indazol-5-ol (i-8) MS (ESI): m/z=317.10 (MH⁺).
Intermediate i-9—(4aR,5R)-4a-Cyclopropyl-5-ethynyl-1-(4-fluorophenyl)-1,4,4a,5,6,7-hexahydrocyclopenta[f]indazol-5-ol (i-9) MS (EST): m/z=335.04 (MH⁺).
Intermediate i-10—(4aR,5R)-4a-Cyclopropyl-5-ethynyl-1-(3-fluorophenyl)-1,4,4a,5,6,7-hexahydrocyclopenta[f]indazol-5-ol i-10 MS (ESI): m/z=335.10 (MH⁺).
Intermediate i-11—(4aR,5R)-4a-cyclopropyl-5-Ethynyl-1-(pyridin-3-yl)-1,4,4a,5,6,7-hexahydrocyclopenta[f]indazol-5-ol (i-11) MS (ESI): m/z=318.20 (MH⁺).

### Intermediate 12

### 2-Iodo-6-methoxybenzamide (i-12)

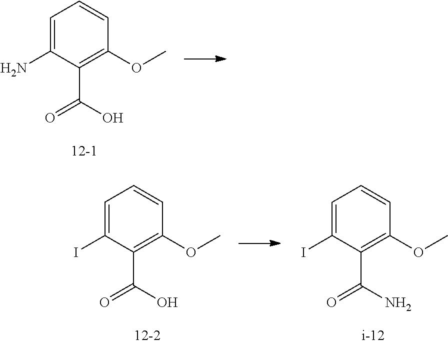

### Step A: 2-Iodo-6-methoxybenzoic acid (12-2)

Sulfuric acid (417 μl, 7.50 mmol) was added to a suspension of 2-amino-6-methoxybenzoic acid (12-1, 209 mg, 1.250 mmol) in water (2501 μl) at 0° C. A solution of sodium nitrite (86 mg, 1.250 mmol) in water (2 mL) was added and stirred for 10 min at 0° C. to afford a yellow solution. Then, a solution of potassium iodide (623 mg, 3.75 mmol) in water (2 mL) was added at 0° C. and the resulting solution immediately turned orange in color as it warmed to ambient temperature. After stirring overnight (15 hrs), the reaction was quenched with 10% w/v aqueous sodium thiosulfate solution and the mixture was extracted with ethyl acetate (3×25 mL). The combined organic fractions were dried (MgSO₄) and filtered and the solvent was evaporated under reduced pressure to give 12-2 (324 mg, 77%) as a crude (83% pure by HPLC) orange solid. MS (ESI): m/z=260.99 (M-18).

### Step B: 2-Iodo-6-methoxybenzamide (i-12)

Dissolved 2-iodo-6-methoxybenzoic acid (12-2) (324 mg, 1.165 mmol), HATU (487 mg, 1.282 mmol), Hunig's base (0.611 mL, 3.50 mmol) and ammonia (2M in dioxane, 4.66 mL, 2.331 mmol) in DMF (8 ml) and let stir for 2 h at ambient temperature. Evaporated off dioxane and DMF and extracted between EtOAc (3×30 mL) and saturated aqueous sodium bicarbonate solution. Dried organic layers (MgSO₄) and then filtered and removed solvent under reduced pressure. The residue was purified by column chromatography on silica gel, eluting 50-100% EtOAc in hexanes to give i-12 (314 mg, 97%) as a white solid. MS (ESI): m/z=260.92 (M-18).

### Intermediates 13-15 (i-13 i-15)

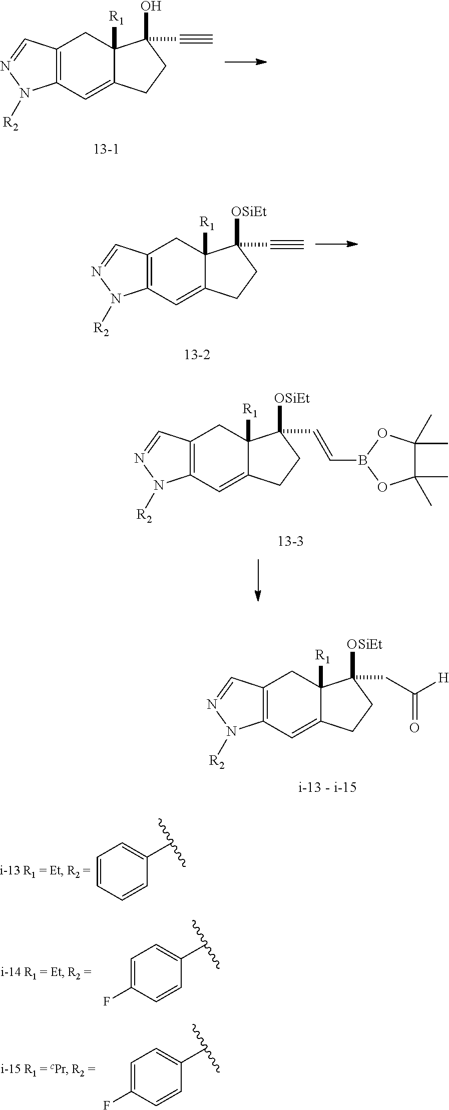

### Step A: (4aS,5R)-4a-Ethyl-5-ethynyl-1-phenyl-5-[(triethylsilyl)oxy]-1,4,4a,5,6,7-hexahydrocyclopenta[f]indazole (13-2)

Triethylsilyltrifluoromethanesulfonate (11.46 g, 43.4 mmol) was added to a stirring solution of 13-1 (12 g, 39.4 mmol) and triethylamine (10.99 ml, 79 mmol) in dichloromethane (79 ml) at room temp. Reaction was complete after addition. Concentrated in vacuo. The residue was purified by column chromatography on silica gel, eluted with 15% ethyl acetate in hexanes to give 13-2 (14 g, 85%) as an orange oil. MS (ESI): m/z=419.3 (MH⁺).

### Step B: (4aS,5R)-4a-Ethyl-1-phenyl-5-[(E)-2-(4,4,5,5-tetramethyl-1,3,2-dioxaborolan-2-yl)ethenyl]-5-[(triethylsilyl)oxy]-1,4,4a,5,6,7-hexahydrocyclopenta[f]indazole 13-3

Pinacolborane (28.7 ml, 28.7 mmol) was added to a solution mixture under nitrogen containing chloro(1,5-cyclooctadiene)rhodium(I) dimer (0.848 g, 1.720 mmol), triisopropylphosphine (0.551 g, 3.44 mmol) and triethylamine (19.98 ml, 143 mmol) in tetrahydrofuran (143 ml). Intermediate 13-2 was added to the reaction mixture (12 g, 28.7 mmol) (2 mL) and heated to 70° C. in an oil bath left stirring for 2 days. 50% conversion. Concentrated in vacuo. The residue was purified by column chromatography on silica gel, eluted with 10% ethyl acetate in hexanes to give 13-3 (6.2 g, 40%) as a yellow oil. MS (EST): m/z=547.6 (MH⁺).

### Step C: (4aS,5R)-4a-Ethyl-1-phenyl-5-[(triethylsilyl)oxy]-1,4,4a,5,6,7-hexahydrocyclopenta[f]indazol-5-yl)acetaldehyde (i-13)

Sodium perborate tetrahydrate (6.98 g, 45.4 mmol) was added to a solution of 13-3 (6.2 g, 11.34 mmol) in THF/Water (1:1) at room temp. Upon completion the reaction mixture was poured into a solution of brine (100 mL.) Extracted with ethyl acetate (100 mL 3×). The combined organic fractions were dried over magnesium sulfate, filtered and concentrated in vacuo to give i-13 (4 g, 81%) as an orange oil. MS (ESI): m/z=437.5 (MH⁺).

Intermediates i-14 and i-15 can be similarly obtained using the appropriate starting materials.

### Intermediates 16-18 (i-16-i-18)

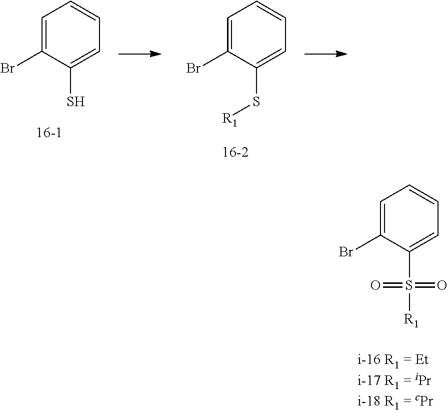

### Step A: 1-Bromo-2-(ethylthio)benzene (16-2)

Added iodoethane (4.95 ml, 61.2 mmol) to a solution of 2-bromobenzenethiol (16-1) (4.63 g, 24.49 mmol) and cesium carbonate (7.98 g, 24.49 mmol) in DMF (5 ml) and let stir at ambient temperature, resulting in considerable exotherm and immediate precipitation of white solid. Reaction was monitored by HPLC and shown to be complete after 2 h. Extracted contents between EtOAc (2×100 mL) and water. The combined organic layers were dried (MgSO₄) and filtered and the solvent was evaporated under reduced pressure to give 16-2 (5.49 g, 100%) as a yellow oil. Used directly in the next reaction

### Step B: 1-Bromo-2-(ethylsulfonyl)benzene (i-16)

Dissolved periodic acid (21.00 g, 92 mmol) in acetonitrile (100 ml) with vigorous stirring for 20 min. Then added chromium(VI) oxide (0.085 ml, 2.303 mmol) and let stir for 5 min until the solution was clear and orange. Finally added 1-bromo-2-(ethylthio)benzene (16-2) (5 g, 23.03 mmol) in acetonitrile (3 mL) slowly via pipette and noticed immediate formation of a white precipitant and significant exotherm. Cooled to 0° C. before adding remainder of (16-2) and then warmed to room temperature after addition was complete. No starting material remained after 2 h of stirring when checked by LC/MS. Filtered off solids through a pad of celite, washed filter cake with acetonitrile (100 mL) and removed solvent with reduced pressure. The residue was purified by column chromatography after first being pre-absorbed onto silica gel (25 g). The contents were then dry-loaded onto a 120 g ISCO cartridge, eluting 0-100% EtOAc in hexanes to afford (i-20) as an orange oil (3.42 g, 60%). MS (ESI): m/z=250.98 (MIT).

Similarly, i-17-i-18 can be prepared from the appropriate starting materials using the procedure similar to that for i-16:
1-Bromo-2-(isopropylsulfonyl)benzene (i-17) MS (ESI): m/z=264.09 (MH⁺).
1-Bromo-2-(cyclopropylsulfonyl)benzene (i-18) MS (ESI): m/z=263.09 (MH⁺).
   These intermediates were used to make final compounds in a manner analogous to Example 2.

### Intermediates 19-21 (i-19-i-21)

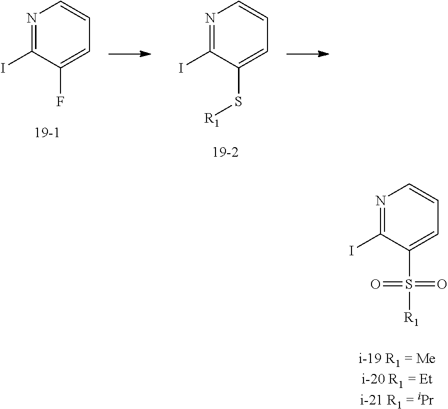

### Step A: 2-Iodo-3-(methylthio)pyridine (19-2)

Added 3-fluoro-2-iodopyridine (102 mg, 0.457 mmol) and sodium methanethiolate (35.3 mg, 0.503 mmol) to a microwave vial and then dissolved contents in DMF (1 ml). Capped the vial and let stir for an hour at room temperature. 95% of starting material was consumed at this time and a little bit of iodide displacement was seen by LC/MS. The contents were partitioned between EtOAc (2×50 mL) and water. The combined organic layers were dried (MgSO₄) and filtered and the solvent was evaporated under reduced pressure. The residue was purified by column chromatography on silica gel, eluting 0-100% EtOAc in hexanes to give 19-2 (54 mg, 47%) as white crystals. MS (ESI): m/z=251.69 (MH⁺).

### Step B: 2-Iodo-3-(methylsulfonyl)pyridine (i-19)

2-Iodo-3-(methylthio)pyridine (19-2) (54 mg, 0.215 mmol) was dissolved in CHCl₃ (1075 μl) at room temperature and to this solution, m-CPBA (101 mg, 0.452 mmol) was added portionwise. Contents were a little cloudy, so more CHCl₃ (1075 μl) was added. Let solution stir overnight. Noticed that it had turned light pink in color after stirring for 10 minutes and then a white precipitate had formed after about 30 minutes. The contents were poured into a saturated solution of aqueous sodium bicarbonate. Extracted with CHCl₃ (2×25 mL). The combined organic layers were dried (MgSO₄) and filtered and the solvent was evaporated under reduced pressure. The residue was purified by column chromatography on silica gel, eluting 10-100% EtOAc in hexanes to give i-19 (47 mg, 68%) as a white solid. MS (ESI): m/z=283.84 (MH⁺).

Similarly, i-20-i-21 can be prepared from the appropriate starting materials using the procedure similar to that for i-19:
3-(Ethylsulfonyl)-2-iodopyridine (i-20) MS (ESI): m/z=297.79 (MH⁺).
2-Iodo-3-(isopropylsulfonyl)pyridine (i-21) MS (ESI): m/z=311.81 (MH⁺).

These intermediates were used to make final compounds in a manner analogous to Example 2, with the exception of Compound 81.

The invention will now be illustrated by the following non-limiting examples in which, unless stated otherwise:

(i) all operations were carried out at room or ambient temperature, that is, at a temperature in the range 18-25° C.,

(ii) evaporation of solvent was carried out using a rotary evaporator under reduced pressure (600-4000 pascals: 4.5-30 mm. Hg) with a bath temperature of up to 60° C.,

(iii) the course of reactions was followed by thin layer chromatography (TLC) and reaction times are given for illustration only;

(iv) melting points are uncorrected and “d” indicates decomposition; the melting points given are those obtained for the materials prepared as described; polymorphism may result in isolation of materials with different melting points in some preparations;

(v) the structure and purity of all final products were assured by at least one of the following techniques: TLC, mass spectrometry, nuclear magnetic resonance (NMR) spectrometry or microanalytical data;

(vi) yields are given for illustration only;

(vii) when given, NMR data is in the form of delta (D) values for major diagnostic protons, given in parts per million (ppm) relative to tetramethylsilane (TMS) as internal standard, determined at 500 MHz or 600 MHz using the indicated solvent; conventional abbreviations used for signal shape are: s. singlet; d. doublet; 1. triplet; m. multiplet; br. broad; etc.: in addition “Ar” signifies an aromatic signal; and

(viii) chemical symbols have their usual meanings.

### Example 1

### 2-{2-[((4aR,5R)-4a-Cyclopropyl-1-(4-fluorophenyl)-5-hydroxy-1,4,4a,5,6,7-hexahydrocyclopenta[f]indazol-5-yl}ethyl}pyridine-3-carboxamide (Ex. 1)

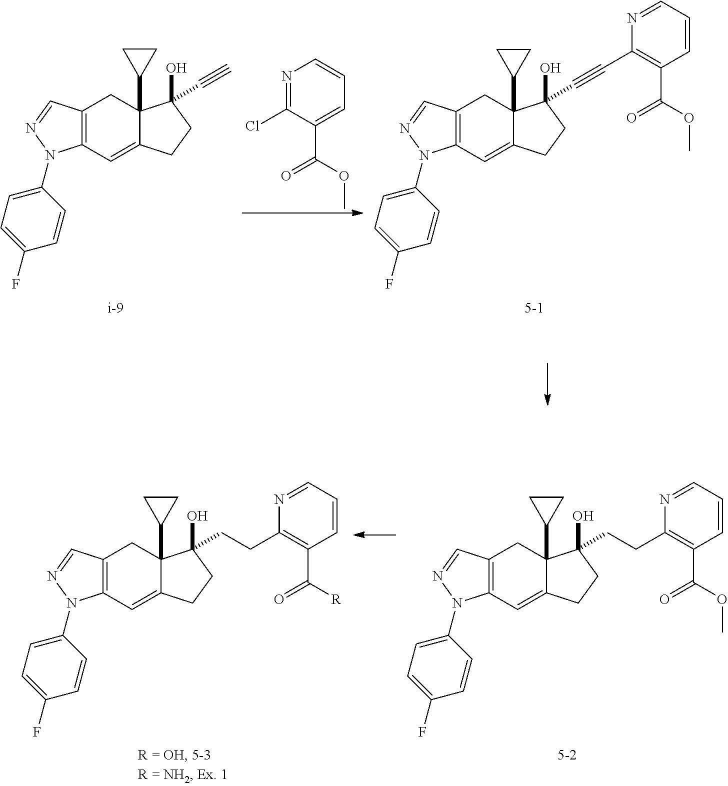

### Step A: Methyl 2-{[((4aR,5R)-4a-cyclopropyl-1-(4-fluorophenyl)-5-hydroxy-1,4,4a,5,6,7-hexahydrocyclopenta[f]indazol-5-yl}ethynyl}pyridine-3-carboxylate (5-1)

Intermediate i-9 (200 mg, 0.598 mmol), methyl 2-chloropyridine-3-carboxylate (123 mg, 0.718 mmol), CuI (5.70 mg, 0.030 mmol) were combined in diisopropylamine (2.99 ml) and then nitrogen was bubbled through the mixture for 10 min. Added tetrakis(triphenylphosphine) palladium (34.6 mg, 0.030 mmol) and then sealed the flask and stirred at 90° C. for 2 h. The reaction was allowed to cool to ambient temperature and then diluted with EtOAc and washed with H₂O, brine, dried (MgSO₄) and then the solvent was evaporated under reduced pressure. The residue was purified by column chromatography on silica gel eluting with hexanes to EtOAc to give 5-1 (223 mg, 79%) as a yellow foam. MS (ESI): m/z=469.9 (MH⁺).

### Step B: 3-Pyridinecarboxylic acid, 2-{2-[((4aR,5R)-4a-cyclopropyl-1-(4-fluorophenyl)-1,4,4a,5,6,7-hexahydro-5-hydroxycyclopenta[f]indazol-5-yl}ethyl]-methyl ester (5-2)

Intermediate 5-1 (220 mg, 0.469 mmol) was dissolved in EtOAc (3 ml), added EtOH (1 ml) followed by 10% Pd/C (200 mg). The mixture was stirred under 1 atm H₂ for 2 h. The reaction was filtered through a celite pad and the solvent was evaporated under reduced pressure to provide 5-2 (210 mg, 95%) of as a yellow foam. MS (ESI): m/z=474.0 (MH⁺).

### Step C: 2-{2-[((4aR,5R)-4a-Cyclopropyl-1-(4-fluorophenyl)-5-hydroxy-1,4,4a,5,6,7-hexahydrocyclopenta[f]indazol-5-yl}ethyl}pyridine-3-carboxylic acid (5-3)

Intermediate 5-2 (200 mg, 0.422 mmol) was dissolved in EtOH (3 ml) and then treated with 1N NaOH (1.0 ml, 1.0 mmol). The mixture was stirred for 2 h. The mixture was concentrated and the residue was dissolved in 1N HCl (1.2 ml, 1.2 mmol) and then concentrated. The residue was azeotroped with THF (3×5 ml) to give 5-3 (194 mg, 100%) as a yellow foam. MS (ESI): m/z=460.1 (MH⁺).

### Step D: 2-{2-[((4aR,5R)-4a-Cyclopropyl-1-(4-fluorophenyl)-5-hydroxy-1,4,4a,5,6,7-hexahydrocyclopenta[f]indazol-5-yl]ethyl}pyridine-3-carboxamide (Ex. 1)

To a stirred solution of 5-3 (100 mg, 0.218 mmol), N-methyl morpholine (88 mg, 0.870 mmol), 0.5 M ammonia in dioxane (0.870 ml, 0.435 mmol) and DMF (2 ml) was added HATU (103 mg, 0.272 mmol). The mixture was stirred for 2 h. The mixture was diluted with EtOAc and then washed with H₂O, sat NaHCO₃, brine, dried (MgSO₄), filtered and the solvent was evaporated under reduced pressure. The residue was purified by column chromatography on silica gel eluting with CHCl₃ to 70:20:10 CHCl₃/EtOAc/MeOH to give Ex. 1 (52 mg, 52.1%) as a yellow foam. MS (ESI): m/z=458.9 (MH⁺). HRMS (ESI): m/z=459.2189 (MH⁺).

### Example 2

### (4aR,5R)-4a-Cyclopropyl-1-(4-fluorophenyl)-5-{2-[2(hydroxymethyl)phenyl]ethyl}-1,4,4a,5,6,7-hexahydrocyclopenta[f]indazol-5-ol (EX. 2)

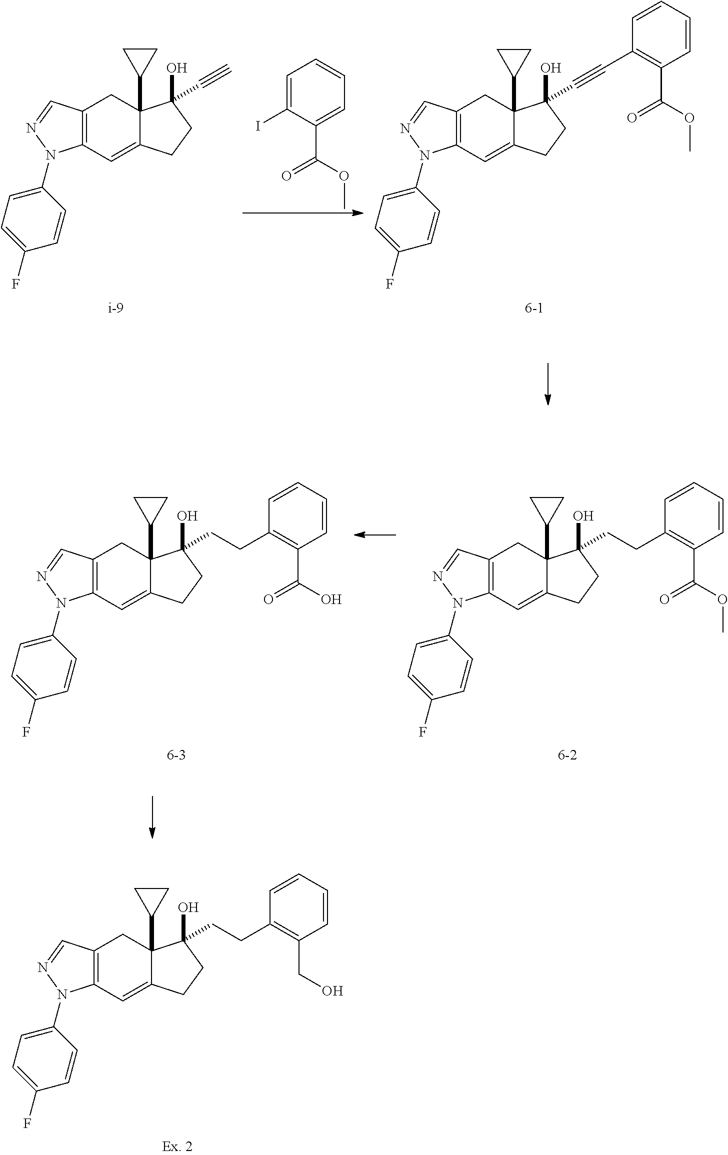

### Step A: Methyl 2-{[((4aR,5R)-4a-cyclopropyl-1-(4-fluorophenyl)-5-hydroxy-1,4,4a,5,6,7-hexahydrocyclopenta[f]indazol-5-yl}ethynyl}benzoate (6-1)

Intermediate i-9 (400 mg, 1.20 mmol), 2-iodobenzoate (376 mg, 1.44 mmol), CuI (11.39 mg, 0.060 mmol), diisopropylamine (0.179 ml, 1.26 mmol) were combined in THF (4 ml) and then nitrogen gas was bubbled through the mixture for 10 min. Added bis(triphenylphosphine)palladium(II) chloride (42.0 mg, 0.060 mmol) and then sealed the flask and stirred at 70° C. for 2 h. The reaction was allowed to cool to ambient temperature and then diluted with Et₂O, filtered through a celite pad and then the solvent was evaporated under reduced pressure. The residue was purified by column chromatography on silica gel eluting with hexanes to EtOAc to give 6-1 (440 mg, 79%) as a yellow foam. MS (ESI): m/z=468.9 (MH⁺).

### Step B: Methyl 2-{2-[((4aR,5R)-4a-cyclopropyl-1-(4-fluorophenyl)-5-hydroxy-1,4,4a,5,6,7-hexahydrocyclopenta[f]indazol-5-yl}ethyl}benzoate (6-1)

Intermediate 6-1 (440 mg, 0.939 mmol) was dissolved in 1:1 EtOAc/EtOH (6 ml), added EtOH (1 ml) added 10% Pd/C (300 mg). The mixture was stirred under 1 atm H₂ for 1 h. The reaction was filtered through a celite pad and the solvent was evaporated under reduced pressure to provide 6-2 (425 mg, 96%) as a yellow foam. MS (ESI): m/z=473.1 (MH⁺).

### Step C: 2-{2-[((4aR,5R)-4a-Cyclopropyl-1-(4-fluorophenyl)-5-hydroxy-1,4,4a,5,6,7-hexahydrocyclopenta[f]indazol-5-yl}ethyl}benzoic acid (6-3)

Intermediate 6-2 (400 mg, 0.846 mmol) was dissolved in EtOH (6 ml) and then treated with 1M NaOH (3.0 ml, 3.0 mmol). The mixture was heated to 60° C. for 2 h. The mixture was allowed to cool to ambient temperature and then was acidified with the addition of 1 N HCl. The mixture was extracted with EtOAc. The organic portion was washed with brine, dried (MgSO₄) and then the solvent was evaporated under reduced pressure to give 6-3 (388 mg, 100%) as a yellow foam. MS (ESI): m/z=459.2 (MH⁺).

### Step D: (4aR,5R)-4a-Cyclopropyl-1-(4-fluorophenyl)-5-{2-[2(hydroxymethyl)phenyl]ethyl}-1,4,4a,5,6,7-hexahydrocyclopenta[f]indazol-5-ol (Ex. 2)

1 M lithium aluminum hydride in THF was added to a stirred, cooled 0° C. mixture of 6-3 (100 mg, 0.218 mmol) in THF (1 mL) and the mixture was allowed to warm to ambient temperature and then for 30 min. The mixture was heated at reflux for 1 h and then the mixture was allowed to cool to ambient temperature. The mixture was diluted with aqueous ammonium chloride (saturated), and extracted with ethyl acetate. The combined organic fractions were washed with brine, dried (MgSO₄), filtered and the solvent was evaporated under reduced pressure. The residue was triturated with EtOAc, the solid was collected, washed with EtOAc (2 ml) and then dried in vacuo to give Ex. 2 (45 mg, 46.4%) as a white solid. MS (ESI): m/z=445.1 (MH⁺).

HRMS (ESI): m/z=445.2285 (MH⁺).

### Example 3a & 3b

### (2S or 2R){2-[2-(2-(4aR,5R)-4a-Cyclopropyl-1-(4-fluorophenyl)-5-hydroxy-1,4,4a,5,6,7-hexahydrocyclopenta[f]indazol-5-yl}ethyl}phenyl}-2-hydroxyethanamide (EX. 3a & 3b)

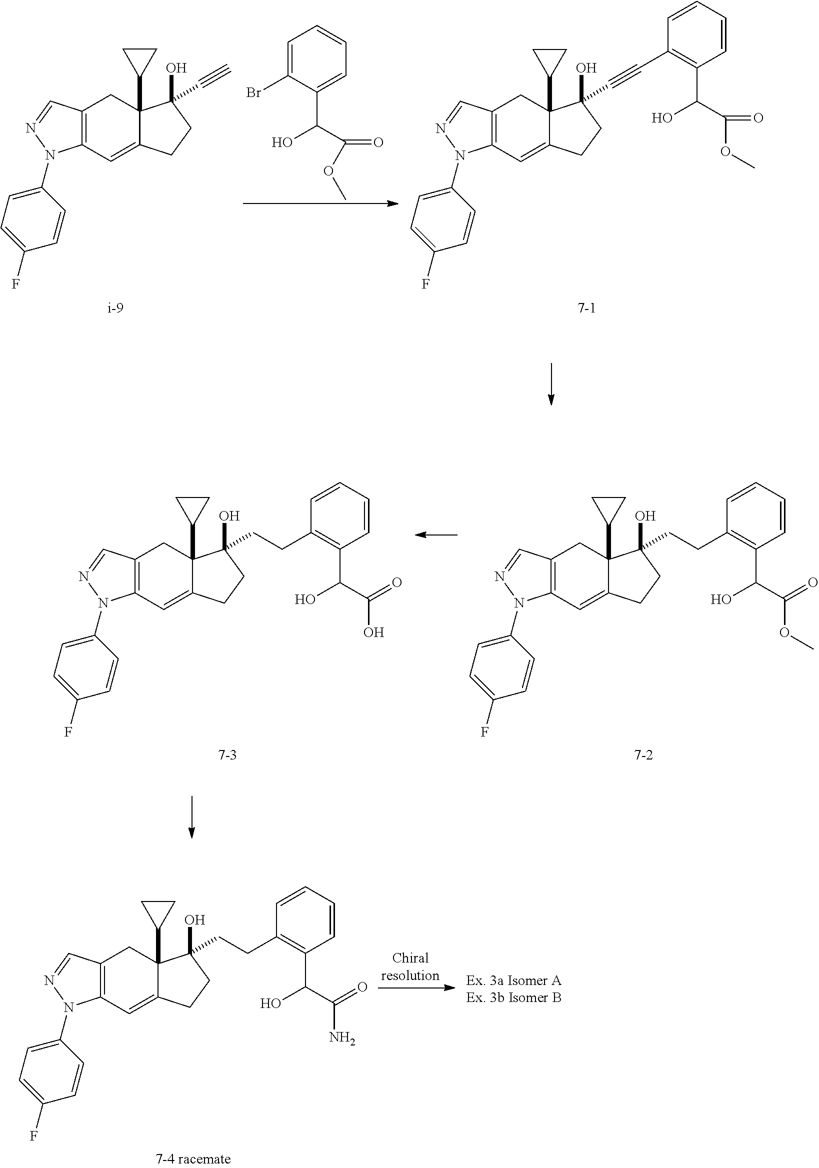

### Step A: Methyl {2-[((4aR,5R)-4a-cyclopropyl-1-(4-fluorophenyl)-5-hydroxy-1,4,4a,5,6,7-hexahydrocyclopenta[f]indazol-5-yl}ethynyl]phenyl}(hydroxy)acetate (7-1)

Intermediate i-9 (400 mg, 1.196 mmol), bromide (366 mg, 1.495 mmol), CuI (11.39 mg, 0.060 mmol) were combined in diisopropylamine (3.99 ml) and then nitrogen was bubbled through the mixture for 10 min. Added tetrakis (69.1 mg, 0.060 mmol) and then sealed the flask and stirred at 90° C. for 2 h. The reaction was allowed to cool to ambient temperature and then the solvent was evaporated under reduced pressure. The residue was purified by column chromatography on silica gel eluting with hexanes to EtOAc to give 7-1 (400 mg, 67.1%) as an orange foam. MS (ESI): m/z=498.9 (MH⁺).

### Step B: Methyl {2-[2-((4aR,5S)-4a-cyclopropyl-1-(4-fluorophenyl)-5-hydroxy-1,4,4a,5,6,7-hexahydrocyclopenta[f]indazol-5-yl}ethyl}phenyl}(hydroxy)acetate (7-2)

7-1 (400 mg, 0.802 mmol) was dissolved in 1:1 EtOAc/EtOH (6.0 ml) followed by 10% Pd/C (300 mg). The mixture was stirred under 1 atm H₂ for 2 h. The reaction was filtered through a celite pad and the solvent was evaporated under reduced pressure to provide 7-2 (390 mg, 97%) of as a yellow foam. MS (ESI): m/z=503.2 (MH⁺).

### Step C: Methyl {2-[2-((4aR,5S)-4a-cyclopropyl-1-(4-fluorophenyl)-5-hydroxy-1,4,4a,5,6,7-hexahydrocyclopenta[f]indazol-5-yl}ethyl}phenyl}(hydroxy)acetic acid (7-3)

7-2 (400 mg, 0.796 mmol) was dissolved in EtOH (3 ml) and then treated with 1N NaOH (1.0 ml, 1.0 mmol). The mixture was heated at 60° C. for 2 h. The mixture was concentrated and the residue was dissolved in 1N HCl (1.1 ml, 1.1 mmol) and then concentrated. The residue was azeotroped with THF (3×10 ml) to give 7-3 (380 mg, 98%) as a yellow foam. MS (ESI): m/z=489.0 (MH⁺).

### Step D: (2S or 2R){2-[2-(2-(4aR,5R)-4a-cyclopropyl-1-(4-fluorophenyl)-5-hydroxy-1,4,4a,5,6,7-hexahydrocyclopenta[f]indazol-5-yl}ethyl]phenyl}-2-hydroxyethanamide (Ex. 3a & Ex. 3b)

To a stirred solution of 7-3 (380 mg, 0.778 mmol), N-methyl morpholine (315 mg, 3.11 mmol), 0.5M ammonia in dioxane (3.11 ml, 1.56 mmol) and DMF (3.9 ml) was added HATU (370 mg, 0.972 mmol). The mixture was stirred for 2 h. The mixture was diluted with EtOAc and then washed with H₂O, sat NaHCO₃, brine, dried (MgSO₄), filtered and the solvent was evaporated under reduced pressure to provide 7-4 racemate. The residue was purified using preparative HPLC Chiralpak AD, eluting with 20% isopropanol/hexanes+0.1% Et₂NH, to provide Ex. 3a (142 mg, 74.9%) as a colorless foam and Ex. 3b (123 mg, 64.9%) as a colorless foam.

MS (ESI): m/z=487.9 (MH⁺). HRMS (ESI): m/z=488.2348 (MH⁺).

MS (ESI): m/z=487.9 (MH⁺). HRMS (ESI): m/z=488.2348 (MH⁺).

### Example 4

### {2-(2(4aR,5R)-4a-Cyclopropyl-1-(4-fluorophenyl)-5-hydroxy-1,4,4a,5,6,7-hexahydrocyclopenta[f]indazol-5-yl}ethyl}-5-fluorobenzamide (Ex. 4)

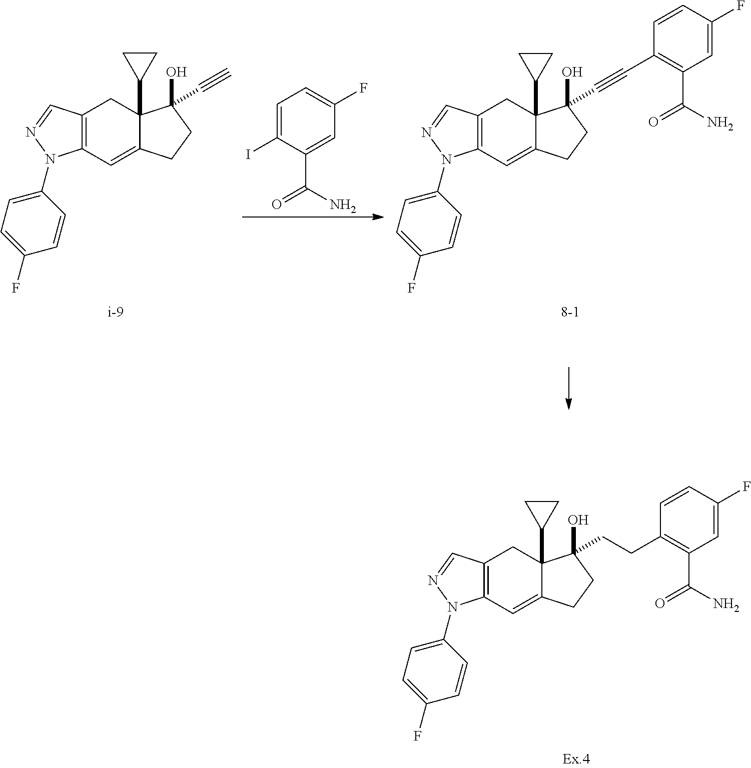

### Step A: {2-((4aR,5R)-4a-Cyclopropyl-1-(4-fluorophenyl)-5-hydroxy-1,4,4a,5,6,7-hexahydrocyclopenta[f]indazol-5-yl}ethynyl}-5-fluorobenzamide (8-1)

Intermediate i-9 (100 mg, 0.299 mmol), 2-iodo-5-fluorobenzamide (103 mg, 0.389 mmol), and piperidine (59.2 μl, 0.598 mmol) were dissolved in acetonitrile (1495 μl) and the solution degassed with vac/nitrogen cycles (×3). Allylpalladium chloride dimer (4.38 mg, 0.012 mmol) and tri-(t-butyl)phosphonium hydrogen HBF₄ salt (17.35 mg, 0.060 mmol) were added and the degassing cycle repeated. The resulting solution was heated at 80° C. for 1 h. The solution was allowed to cool to ambient temperature and then the solvent was evaporated under reduced pressure. The residue was purified by column chromatography on silica gel eluting with hexanes to EtOAc to give 8-1 (123 mg, 87%) as a colorless foam. MS (ESI): m/z=471.9 (MH⁺).

### Step B: {2-(2(4aR,5R)-4a-Cyclopropyl-1-(4-fluorophenyl)-5-hydroxy-1,4,4a,5,6,7-hexahydrocyclopenta[f]indazol-5-yl}ethyl}-5-fluorobenzamide (Ex. 4)

Intermediate 8.4 (115 mg, 0.244 mmol) was dissolved in 1:1 EtOAc/EtOH (2.4 ml) followed by 10% Pd/C (100 mg). The mixture was stirred under 1 atm H₂ for 2 h. The reaction was filtered through a celite pad and the solvent was evaporated under reduced pressure. The residue was purified by column chromatography on silica gel ISCO 12 gram, eluting with hexanes to EtOAc to give Ex. 4 (93 mg, 80%) as a colorless foam. MS (ESI): m/z=475.9 (MH⁺).

HRMS (ESI): m/z=476.2133 (MH⁺).

### Example 5

### {2-(2(4aS,5R)-4a-Ethyl-5-hydroxy-1-(pyridin-3-yl)-1,4,4a,5,6,7-hexahydrocyclopenta[f]indazol-5-yl}ethyl}-benzonitrile (Ex. 5)

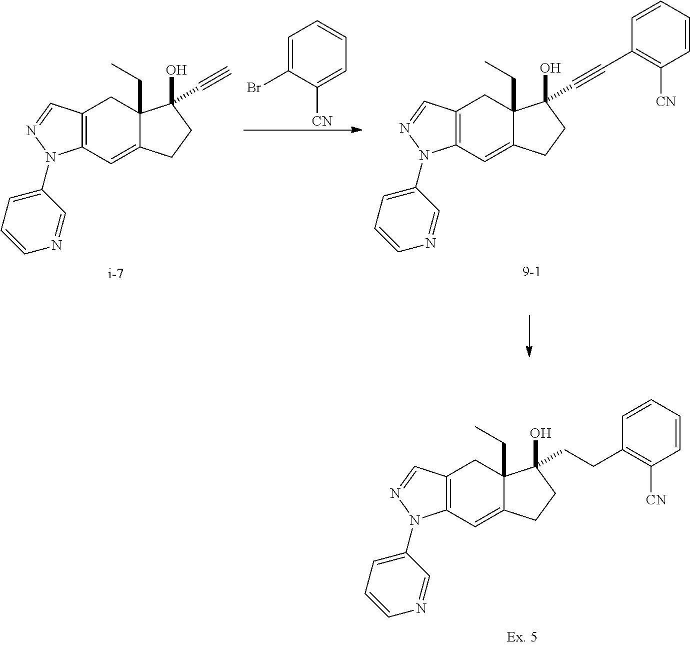

### Step A: {2-((4aS,5R)-4a-Ethyl-5-hydroxy-1-(pyridin-3-yl)-1,4,4a,5,6,7-hexahydrocyclopenta[f]indazol-5-yl}ethynyl}-benzonitrile (9-1)

Intermediate i-7 (250 mg, 0.819 mmol), 2-bromobenzonitrile (224 mg, 1.23 mmol), CuI (3.9 mg, 0.020 mmol), diisopropylamine (0.123 ml, 0.860 mmol) were combined in THF (2 ml) and then nitrogen gas was bubbled through the mixture for 10 min. Added bis(triphenylphosphine)palladium(II) chloride (14.4 mg, 0.020 mmol) and then sealed the flask and stirred at 70° C. for 2 h. The reaction was allowed to cool to ambient temperature and then was diluted with Et2O, filtered through a celite pad and then the solvent was evaporated under reduced pressure. The residue was triturated with CH₂Cl₂, solid collected and dried in vacuo to give 9-1 (201 mg, 60.4%) as a yellow solid. MS (ESI): m/z=407.06 (MH⁺).

### Step B: {2-(2-(4aS,5R)-4a-Ethyl-5-hydroxy-1-(pyridin-3-yl)-1,4,4a,5,6,7-hexahydrocyclopenta[f]indazol-5-yl}ethyl}-benzonitrile (Ex. 5)

Intermediate 9-1 (200 mg, 0.492 mmol) was dissolved in EtOAc (5.0 ml) followed by 10% Pd/C (200 mg). The mixture was stirred under 1 atm H₂ at 45° C. for 2 h. The mixture was allowed to cool to ambient temperature and then was filtered through a celite pad and the solvent was evaporated under reduced pressure. The residue was purified by column chromatography on silica gel eluting with hexanes to EtOAc to give Ex. 5 (120 mg, 59.4%) as a colorless foam. MS (ESI): m/z=411.19 (MH⁺). HRMS (ESI): m/z=411.2186 (MH⁺).

### Example 6

### 2-(2-(4aS,5R)-4a-Ethyl-5-hydroxy-1-phenyl-1,4,4a,5,6,7-hexahydrocyclopenta[f]indazol-5-yl}ethyl}-5-fluorobenzamide (Ex. 6)

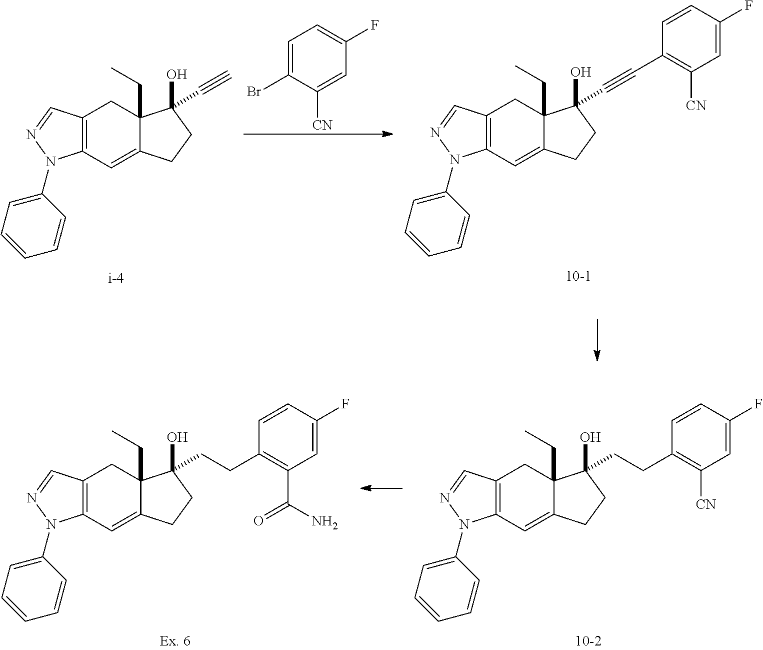

### Step A: 2-((4aS,5R)-4a-Ethyl-5-hydroxy-1-phenyl-1,4,4a,5,6,7-hexahydrocyclopenta[f]indazol-5-yl}ethynyl}-5-fluorobenzonitrile (10-1)

Intermediate i-4 (300 mg, 0.986 mmol), 2-bromo-5-fluorobenzonitrile (296 mg, 1.48 mmol), CuI (4.69 mg, 0.025 mmol), diisopropylamine (0.147 ml, 1.035 mmol) were combined in THF (2 ml) and then nitrogen gas was bubbled through the mixture for 10 min. Added bis(triphenylphosphine)palladium(II) chloride (17.29 mg, 0.025 mmol) and then sealed the flask and stirred at 70° C. for 2 h. The reaction was allowed to cool to ambient temperature and then was diluted with Et2O, filtered through a celite pad and then the solvent was evaporated under reduced pressure. The residue was purified by column chromatography on silica gel ISCO 12 gram, eluting with hexanes to EtOAc to give 10-1 (220 mg, 52.7%) as an orange oil. MS (ESI): m/z=423.9 (MH⁺).

### Step B: 2-(2-(4aS,5R)-4a-Ethyl-5-hydroxy-1-phenyl-1,4,4a,5,6,7-hexahydrocyclopenta[f]indazol-5-yl}ethyl}-5-fluorobenzonitrile (10-2)

Intermediate 10-1 (100 mg, 0.235 mmol) was suspended in EtOAc (4.0 ml), added EtOH (1.0 ml) for solubility, followed by 10% Pd/C (100 mg). The mixture was stirred under 1 atm H2 for 2 h. The mixture was filtered through a celite pad and the solvent was evaporated under reduced pressure. The residue was purified by column chromatography on silica gel ISCO 12 gram, eluting with hexanes to EtOAc to give 10-2 (95 mg, 94%) as a colorless foam. MS (ESI): m/z=428.15 (MH⁺).

### Step C: 2-(2-(4aS,5R)-4a-Ethyl-5-hydroxy-1-phenyl1,4,4a,5,6,7-hexahydrocyclopenta[f]indazol-5-yl}ethyl}-5-fluorobenzamide (Ex. 6)

To a solution of 10-2 (75 mg, 0.175 mmol) in IPA (3 ml), was added 25% NaOH (1 ml). The mixture was heated at 150° C. for 45 min on a microwave reactor. The reaction was quenched with the addition of 1 M HCl and then the mixture was extracted with ethyl acetate. The organic portion was washed with brine, dried (MgSO₄), filtered and the solvent was evaporated under reduced pressure. The residue was purified by column chromatography on silica gel ISCO 12 gram, eluting with hexane to EtOAc to give Ex. 6 (48 mg, 61.4%) as a colorless foam.

MS (ESI): m/z=446.21 (MH⁺). HRMS (ESI): m/z=446.2241 (MH⁺).

### Example 7

### {2-(2-(4aR,5R)-4a-Cyclopropyl-1-(3-fluorophenyl)-5-hydroxy-1,4,4a,5,6,7-hexahydrocyclopenta[f]indazol-5-yl}ethyl}-benzamide (Ex. 7)

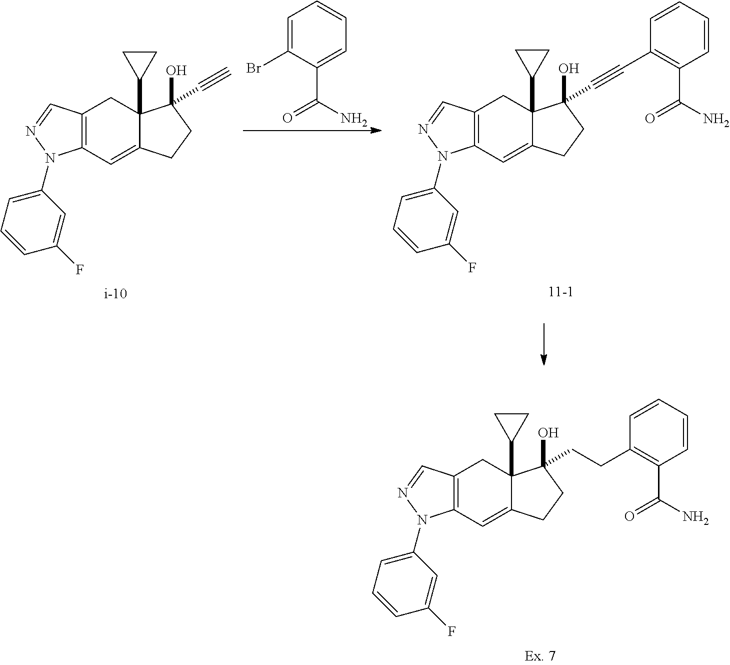

### Step A: {2-((4aR,5R)-4a-Cyclopropyl-1-(3-fluorophenyl)-5-hydroxy-1,4,4a,5,6,7-hexahydrocyclopenta indazol-5-yl}ethynyl}-benzamide (11-1)

Intermediate i-10 (100 mg, 0.299 mmol), Reactant 2 (78 mg, 0.389 mmol), and piperidine (59.2 μl, 0.598 mmol) were dissolved in acetonitrile (1495 μl) and the solution degassed with vac/nitrogen cycles (×3). allylpalladium chloride dimer (4.38 mg, 0.012 mmol) and tri-(t-butyl)phosphonium hydrogen HBF₄ salt (17.35 mg, 0.060 mmol) were added and the degassing cycle repeated. The resulting solution was heated at 80° C. for 1 h. The solution was allowed to cool to ambient temperature and then the solvent was evaporated under reduced pressure. The residue was purified by column chromatography on silica gel ISCO 12 gram, eluting with hexanes to EtOAc to give 11-1 (65 mg, 47.9%) as a yellow foam.

MS (ESI): m/z=454.2 (MH⁺).

### Step B: {2-(2-(4aR,5R)-4a-Cyclopropyl-1-(3-fluorophenyl)-5-hydroxy-1,4,4a,5,6,7-hexahydrocyclopenta[f]indazol-5-yl}ethyl}-benzamide (Ex. 7)

Intermediate 11-1 (75 mg, 0.165 mmol) was dissolved in 1:1 EtOAc/EtOH (2.0 ml) followed by 10% Pd/C (100 mg). The mixture was stirred under 1 atm H₂ for 2 h. The reaction was filtered through a celite pad and the solvent was evaporated under reduced pressure. The residue was purified by column chromatography on silica gel ISCO 12 gram, eluting with hexanes to EtOAc to give Ex. 7 (70 mg, 93%) as a colorless foam. MS (ESI): m/z=458.3 (MH⁺).

HRMS (ESI): m/z=458.2245 (MH⁺).

### Examples 37 and 38 (Ex. 37 and Ex. 38)

Examples 37 and 38 were prepared following procedures similar to Example 4 using intermediate 2-iodo-6-methoxybenzamide (i-12):
2-{2-[(4aS,5R)-4a-Ethyl-1-(4-fluorophenyl)-5-hydroxy-1,4,4a,5,6,7hexahydrocyclopenta[f]indazol-5-yl]ethyl}-6-methoxybenzamide (Ex. 37) MS (ESI): m/z=475.9 (MH⁺). HRMS (ESI): m/z=476.2339 (MH⁺).
2-{2-[(4aR,5R)-4a-Cyclopropyl-1-(4-fluorophenyl)-5-hydroxy-1,4,4a,5,6,7-hexahydrocyclopenta[f]indazol-5-yl]ethyl}-6-methoxybenzamide (Ex. 38) MS (ESI): m/z 487.9 (MH⁺). HRMS (EST): m/z=488.2341 (MH⁺).

### Example 57

### (2R)-2-(2-{2-[(4aR,5R)-4a-Cyclopropyl-1-(4-fluorophenyl)-5-hydroxy-1,4,4a,5,6,7-hexahydrocyclopenta[f]indazol-5-yl]ethyl}-6-fluorophenyl)-2-fluoroethanamide (Ex. 57)

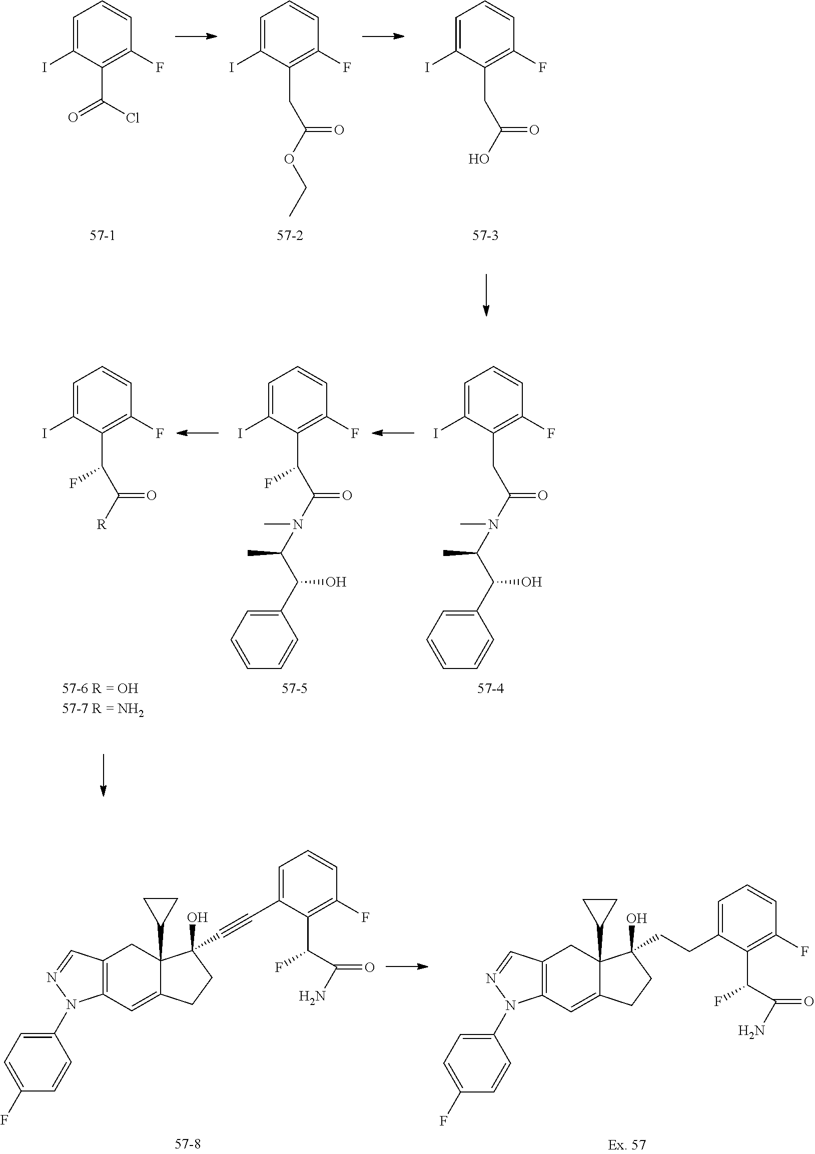

### Step A: Ethyl (2-fluoro-6-iodophenyl)acetate (57-2)

TMS-diazomethane solution (65.9 ml, 2.0 M in diethyl ether, 132 mmol) was added to 200 ml 1:1 THF/CH₃CN and then cooled to 0° C. NEt₃ (18.4 ml, 132 mmol) was added and then 2-fluoro-6-iodobenzoyl chloride was added dropwise over 20 minutes. Stirred for 1 hour at 0° C. and then stored in freezer (−4° C.) overnight. Diluted with EtOAc and then washed with H₂O, 0.1 N HCl, brine, dried (MgSO₄) and concentrated. Dissolved residue in EtOH (100 ml), added NEt₃ (14.7 ml, 106 mmol) followed by portionwise addition of silver benzoate (3.02 g, 13.2 mmol, gas evolution). Heated to 80° C. for 10 minutes and then allowed to cool to ambient. The mixture was filtered through a celite pad and then the solution was concentrated. The residue was purified by column chromatography on silica gel, eluting with hexanes to 20% EtOAc/hexanes to give 57-2 (21.0 g, 78%) as a colorless oil. MS (ESI): m/z=308.8 (MH⁺).

### Step B: (2-Fluoro-6-iodophenyl)acetic acid (57-3)

Intermediate 57-2 (20 g, 64.9 mmol) was dissolved in EtOH (200 ml), added 100 ml 1N NaOH and then heated to 50° C. for 1 hour. The reaction was allowed to ambient temp, acidified with 1 N HCl and then the mixture was extracted with EtOAc. The organic portion was washed with brine, dried (MgSO₄) and concentrated to give 57-3 (18.5 g, quant) as a yellow solid. Used directly in the next step.

### Step C: 2-(2-Fluoro-6-iodophenyl)-N-[(1R,2R)-1-hydroxy-1-phenylpropan-2-yl]-N-methylacetamide (57-4)

To a stirred solution of 57-3 (18.5 g, 66.1 mmol), (1R,2R)-(−)-pseudoephedrine (16.37 g, 99 mmol), N-methylmorpholine (29.1 ml, 264 mmol) and DMF (50 ml) was added HATU (26.4 g, 69.4 mmol). The reaction was stirred for 16 hours. The mixture was diluted with EtOAc and then washed with H₂O, sat NaHCO₃, brine, dried (MgSO₄), filtered and the solvent was evaporated under reduced pressure. The residue was purified by column chromatography on silica gel, eluting with hexanes to EtOAc to give 57-4 (18.0 g, 63.8%) as a colorless solid. MS (ESI): m/z=428.0 (MH⁺).

### Step D: (2R)-2-Fluoro-2-(2-fluoro-6-iodophenyl)-N-[(1R,2R)-1-hydroxy-1-phenylpropan-2-yl]-N-methylethanamide (57-5)

LiCl (7.94 g, 187 mmol) was dried at 120° C. for 20 minutes. LiCl was suspended in THF (117 ml). Added LDA solution (38.6 ml, 2.0M) and stirred 5 minutes. Cooled to −78° C. Intermediate 57-4 (10.0 g, 23.41 mmol) was dissolved in 50 ml THF and then was added dropwise over 5 minutes to the LDA solution. Stirred for 30 minutes. Allowed to warm to −20° C. for 5 minutes and then recooled to −78° C. N-Fluorobenzenesulfonimide (8.12 g, 25.7 mmol), dissolved in 50 ml THF was added, the mixture was stirred for 15 minutes and then quenched with sat. NH₄Cl and then extracted with EtOAc. The organic portion was washed with brine, dried (MgSO₄) and concentrated. The residue was purified by column chromatography on silica gel, eluting with hexanes to EtOAc to give 57-5 (4.25 g, 40.8%) as a yellow solid. MS (ESI): m/z=445.9 (MH⁺).

### Step E: (2R)-Fluoro(2-fluoro-6-iodophenyl)ethanoic acid (57-6)

Intermediate 57-5 (5.0 g, 11.23 mmol) was dissolved in dioxane (56.1 ml), added 30 ml 18N H₂SO₄ and then heated to 120° C. for 30 minutes. The reaction was allowed to cool to ambient temp. Diluted with 100 ml H₂O and then extracted with CH₂Cl₂. The organic portion was washed with brine, dried (MgSO₄) and concentrated to give 57-6 (3.4 g, quant) as a yellow oil. Used directly in the next reaction.

### Step F: (2R)-2-Fluoro-2-(2-fluoro-6-iodophenyl)ethanamide (57-7)

To a stirred solution of 57-6 (3.25 g, 10.91 mmol), NH₃ (26.2 ml, 0.5 M dioxane), N-methylmorpholine (2.40 ml, 21.81 mmol) and DMF (54.5 ml) was added HATU (4.35 g, 11.45 mmol). The reaction was stirred for 16 hours. The mixture was diluted with EtOAc and then washed with H₂O, sat NaHCO₃, brine, dried (MgSO₄), filtered and the solvent was evaporated under reduced pressure. The residue was purified by column chromatography on silica gel, eluting with hexanes to EtOAc to give 57-7 (2.25 g, 69.5%) as a yellow solid. MS (ESI): m/z=297.9 (MH⁺).

### Step G: (2R)-2-(2-{[(4aR,5R)-4-Cyclopropyl-1-(4-fluorophenyl)-5-hydroxy-1,4,4a,5,6,7-hexahydrocyclopenta[f]indazol-5-yl]ethynyl}-6-fluorophenyl)-2-fluoroethanamide (57-8)

Intermediate i-5 (0.150 g, 0.449 mmol), intermediate 57-7 (0.147 g, 0.493 mmol), diisopropyl amine (0.079 ml, 0.561 mmol), CuI (8.54 mg, 0.045 mmol) and THF (1.1 ml) was combined and then degassed with N₂ for 10 minutes. Added Bis(triphenylphosphine)palladium(II) chloride (31.5 mg, 0.045 mmol), sealed flask and heated to 70° C. for 1 hour. The mixture was allowed to cool to ambient temperature and was diluted with EtOAc and then washed with H₂O, brine, dried (MgSO₄) and concentrated. The residue was purified by column chromatography on silica gel, eluting with hexanes to EtOAc to give 57-8 (0.190 g, 84%) as a yellow solid. MS (ESI): m/z=503.9 (MH⁺).

### Step H: (2R)-2-(2-{2-[(4aR,5R)-4a-Cyclopropyl-1-(4-fluorophenyl)-5-hydroxy-1,4,4a,5,6,7-hexahydrocyclopenta[f]indazol-5-yl]ethyl}-6-fluorophenyl)-2-fluoroethanamide (Ex. 57)

Intermediate 57-8 (0.175 g, 0.348 mmol) was dissolved in 1:1 EtOAc/EtOH (4 ml), added 0.2 gram of 10% Pd/C and then stirred under 2 atm H₂ for 1 hour. The reaction mixture was filtered through a celite pad and concentrated. The residue was purified by column chromatography on silica gel, eluting with CHCl₃ to 70:20:10 CHCl₃/EtOAc/MeOH to give Ex. 57 (0.094 g, 53.3%) as a colorless foam. MS (ESI): m/z=508.2 (MH⁺). HRMS (ESI): m/z=508.2194 (MH⁺).

### Example 64

### 2-(2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)acetamide (Ex. 64)

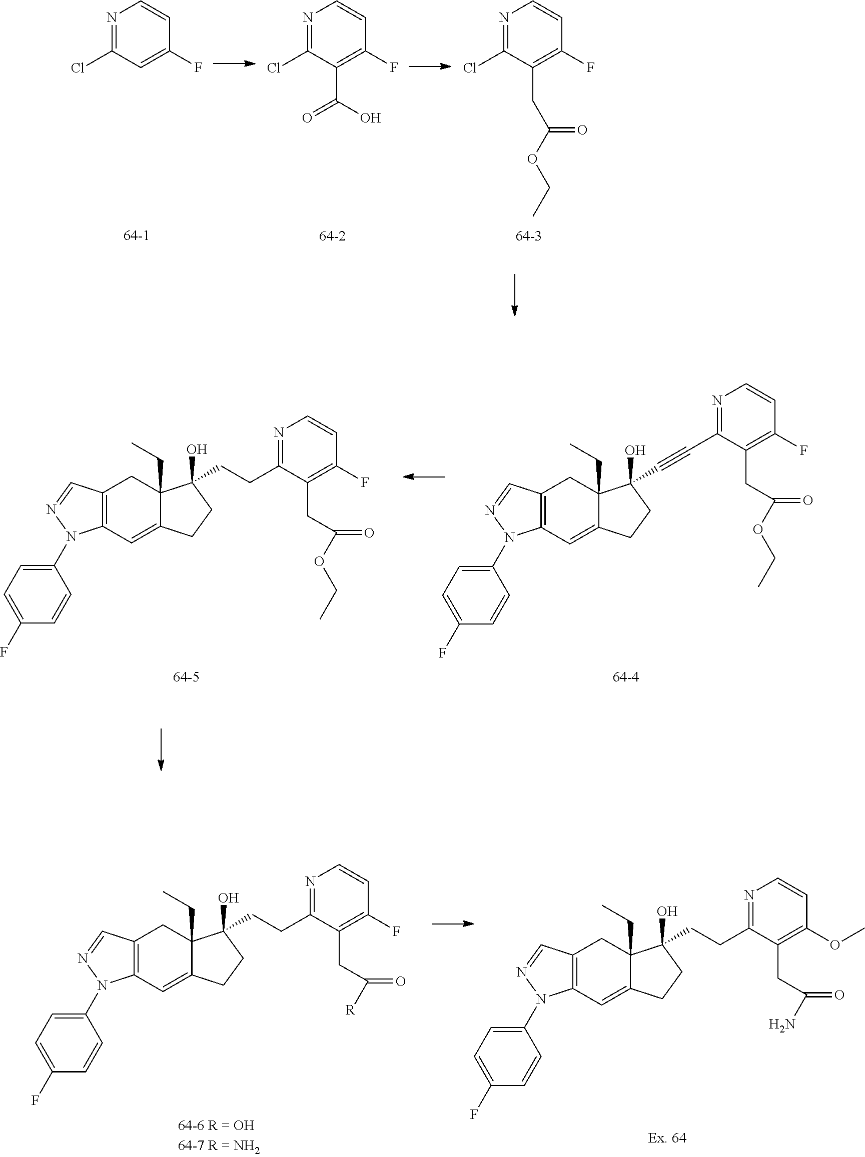

### Step A: 2-Chloro-4-fluoropyridine-3-carboxylic acid (64-2)

A solution of LDA (97 ml, 1.8 M heptane/THF/ethylbenzene, 175 mmol) and THF (304 ml) were combined and cooled to −78° C. 2-Chloro-4-fluoropyridine (20.0 g, 152 mmol) was dissolved in 50 ml THF and then added dropwise over 20 minutes and then the solution was stirred for 1 hour. The reaction was poured onto dry ice and the dry ice was allowed to evaporate. Water and 1 N NaOH were added to the residue. The mixture was extracted with Et₂O and the organic layer discarded. The aqueous was acidified with 1 N HCl and then extracted with EtOAc. The organic portion was washed with brine, dried (MgSO₄) and concentrated to give 64-2 (19 g, 71.2%) as an orange solid. MS (ESI): m/z=176.0 (MH⁺).

### Step B: Ethyl (2-chloro-4-fluoropyridin-3-yl)acetate (64-3)

Intermediate 64-2 (19.9 g, 108 mmol) was dissolved in CH₂Cl₂ (100 ml), added 1 ml DMF and then added oxalyl chloride (17.2 g, 135 mmol) dropwise over 10 minutes. When gas evolution subsided (about an hour), the reaction was concentrated and azeotroped with THF (3×50 ml) to give the crude acid chloride. TMS-diazomethane solution (97 ml, 2.0 M, 195 mmol) was added to 300 ml 1:1 THF/CH₃CN and then cooled to 0° C. NEt₃ (27.2 ml, 195 mmol) was added and then the acid chloride was added dropwise over 20 minutes. Stirred for 1 hour at 0° C. and then stored in freezer (−4° C.) overnight. Diluted with EtOAc and then washed with H₂O; added 0.5 N HCl to organic portion, stirred 5 minutes, basified with 1 N NaOH. Organic portion separated, washed with brine, dried (MgSO₄) and concentrated. Dissolved residue in EtOH (300 ml), added NEt₃ (18.1 ml, 130 mmol) followed by portionwise addition of silver benzoate (3.72 g, 16.24 mmol, gas evolution). Heated to 80° C. for 10 minutes and then allowed to cool to ambient. The mixture was concentrated. Diluted with CH₂Cl₂, filtered and concentrated. The residue was purified by column chromatography on silica gel, eluting with hexanes to EtOAc to give 64-3 (8.05 g, 34.2%) as a colorless oil. MS (ESI): m/z=218.0 (MH⁺).

### Step C: Ethyl (2-{[(4aS,5R)-4a-ethyl-1-(4-fluorophenyl)-5-hydroxy-1,4,4a,5,6,7-hexahydrocyclopenta[f]indazol-5-yl]ethynyl}-4-fluoropyridin-3-yl)acetate (64.4)

Intermediate i-5 (1.0 g, 3.1 mmol), intermediate 64-3 (0.709 g, 3.26 mmol), diisopropyl amine (12.5 ml), and CuI (5.91 mg, 0.031 mmol) was combined and then degassed with N₂ for 10 minutes. Added tetrakis(triphenylphosphine) palladium (36 mg, 0.031 mmol), sealed flask and heated to 90° C. for 2 hours. The mixture was allowed to cool to ambient temperature and was diluted with CH₂Cl₂ and then washed with H₂O, brine, dried (MgSO₄) and concentrated. The residue was purified by column chromatography on silica gel, eluting with hexanes to EtOAc to give 64-4 (1.37 g, 88%) as a yellow foam. MS (ESI): m/z=504.4 (MH⁺).

### Step D: Ethyl (2-{2-[(4aS,5R)-4a-ethyl-1-(4-fluorophenyl)-5-hydroxy-1,4,4a,5,6,7-hexahydrocyclopenta[f]indazol-5-yl]ethyl}-4-fluoropyridin-3-yl)acetate (64-5)

Intermediate 64-4 (1.35 g, 2.68 mmol) was dissolved in 1:1 EtOAc/EtOH (10.7 ml), added 1 gram of 10% Pd/C and then stirred under 1 atm H₂ for 1 hour. The reaction was filtered through a celite pad and concentrated to give 64-5 (1.25 g, 92%) as a yellow foam. MS (ESI): m/z=508.4 (MH⁺).

### Step E: (2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-fluoropyridin-3-yl)acetic acid (64-6)

Intermediate 64-5 (1.25 g, 2.46 mmol) was dissolved in EtOH (9.85 ml), added 3 ml 1N NaOH and then stirred for 2 hours. The reaction was concentrated, added 3.1 ml 1N HCl, concentrated and then azeotroped with THF (3×50 ml) to give 64-6 (1.2 g, quant) as a yellow foam. MS (EST): m/z=480.4 (MH⁺).

### Step F: 2-(2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-fluoropyridin-3-yl)acetamide (64-7)

To a stirred solution of 64-6 (250 mg, 0.521 mmol), ammonia (2.09 ml, 0.5 M dioxane), N-methylmorpholine (0.229 ml, 2.09 mmol) and DMF (2 ml) was added HATU (238 mg, 0.626 mmol). The reaction was stirred for 16 hours. The mixture was diluted with EtOAc and then washed with H₂O, sat NaHCO₃, brine, dried (MgSO₄), filtered and the solvent was evaporated under reduced pressure. The residue was purified by column chromatography on silica gel, eluting with CHCl₃ to 70:20:10 CHCl₃/EtOAc/MeOH to give 64-7 (180 mg, 72.1%) as a colorless foam. MS (EST): m/z=479.3 (MH⁺).

### Step G: 2-(2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)acetamide (Ex. 64)

Intermediate 64-7 (125 mg, 0.261 mmol) was dissolved in anhydrous MeOH (1.3 ml), added sodium methoxide (70.6 mg, 1.31 mmol), and then heated to 60° C. for 3 hours. Quenched with H₂O and then extracted with CH₂Cl₂. The organic portion was dried (MgSO₄) and concentrated. The residue was purified by column chromatography on silica gel, eluting with CHCl₃ to 70:20:10 CHCl₃/EtOAc/MeOH to give Ex. 64 (49 mg, 38.2%) as a colorless foam. MS (ESI): m/z=491.3 (MH⁺). HRMS (ESI): m/z=491.2440 (MH⁺).

### Examples 65a and 65b

### 2-(2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)-2-fluoroacetamide (Ex. 65a and Ex. 65b)

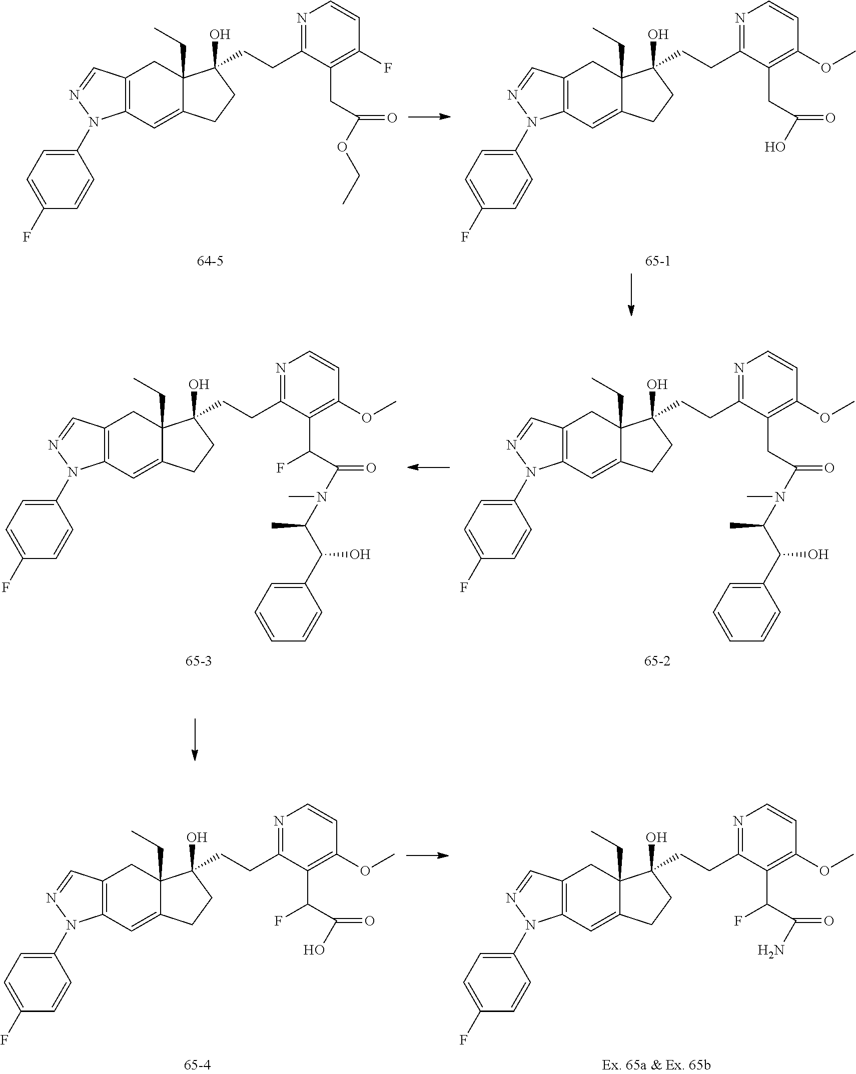

### Step A: (2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)acetic acid (65-1)

Intermediate 64-5 (1.0 g, 1.97 mmol) dissolved in anhydrous MeOH (26.3 ml), added sodium methoxide (1.06 g, 19.7 mmol), and then heated to 60° C. overnight. The reaction was allowed to cool to ambient temp. Added 10 ml H₂O and then acidified with addition of cone HCl. The mixture was concentrated and then azeotroped with THF (3×50 ml) to give 65-1 (1.2 g, quant) as a yellow foam. MS (EST): m/z=492.7 (MH⁺).

### Step B: 2-(2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)-N-[(1R,2R)-1-hydroxy-1-phenylpropan-2-yl]-N-methylacetamide (65-2)

To a stiffed solution of 65-1 (0.96 g, 1.95 mmol), (1R,2R)-(−)-pseudoephedrine (0.484 g, 2.93 mmol), N-methylmorpholine (0.859 ml, 7.81 mmol) and DMF (9.8 ml) was added HATU (1.11 g, 2.93 mmol). The reaction was stirred for 16 hours. The mixture was diluted with EtOAc and then washed with H₂O, sat NaHCO₃, brine, dried (MgSO₄), filtered and the solvent was evaporated under reduced pressure. The residue was purified by column chromatography on silica gel, eluting with CHCl₃ to 70:20:10 CHCl₃/EtOAc/MeOH to give 65-2 (0.83 g, 66.5%) as a colorless foam. MS (ESI): m/z=639.4 (MH⁺).

### Step C: 2-(2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)-2-fluoro-N-[(1R,2R)-1-hydroxy-1-phenylpropan-2-yl]-N-methylacetamide (65-3)

LiCl (0.435 g, 10.27 mmol) was dried at 120° C. for 20 minutes. LiCl was suspended in THF (7.8 ml). Added a solution of LDA (2.5 ml, 1.8M in heptane/THF/ethylbenzene) and stirred 5 minutes. Cooled to −78° C. Intermediate 65-2 (0.820 g, 1.28 mmol) was dissolved in 5 ml THF and then was added dropwise over 5 minutes to the LDA solution. Stirred for 30 minutes. Allowed to warm to −20° C. for 5 minutes and then recooled to −78° C. N-fluorobenzenesulfonimide (0.445 g, 1.41 mmol), dissolved in 5 ml THF was added, the mixture was stirred for 15 minutes and then quenched with sat. NH₄Cl and then extracted with EtOAc. The organic portion was washed with brine, dried (MgSO₄) and concentrated. The residue was purified by column chromatography on silica gel, eluting with CHCl₃ to 70:20:10 CHCl₃/EtOAc/MeOH to give 65-3 (298 mg, 35.3%) as a colorless foam (dr 9:1). MS (ESI): m/z=657.4 (MH⁺).

### Step D: (2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)fluoroacetic acid (65-4)

Intermediate 65-3 (0.298 g, 0.454 mmol) was dissolved in EtOH (2.3 ml), added 2 ml 1N NaOH and then heated to 60° C. for 3 hours, Diluted with H₂O and then extracted with EtOAc. The aqueous layer was collected, acidified with 1 N HCl and then concentrated and azeotroped with THF (3×20 ml) to give 65-4 (235 mg, quant) as a colorless foam (1:1 mixture of isomers). MS (ESI): m/z=510.3 (MH⁺).

### Step E: 2-(2-{2-[(4aS,5R)-4a-Ethyl-1-(4-fluorophenyl)-5-hydroxy-1,4,4a,5,6,7-hexahydrocyclopenta[f]indazol-5-yl]ethyl}-4-methoxypyridin-3-yl)-2-fluoroacetamide (Ex. 65)

To a stirred solution of 65-4 (0.232 g, 0.455 mmol), NH₃ (1.82 ml, 0.5M in dioxane), N-methylmorpholine (0.200 ml, 1.82 mmol) and DMF (5 ml) was added HATU (0.208 g, 0.546 mmol). The reaction was stirred for 16 hours. The mixture was diluted with EtOAc and then washed with H₂O, sat NaHCO₃, brine, dried (MgSO₄), filtered and the solvent was evaporated under reduced pressure. The residue was purified by column chromatography on silica gel, eluting with CHCl₃ to 70:20:10 CHCl₃/EtOAc/MeOH to give the racemic amide (190 mg) as a colorless oil. The isomers were resolved on Chiral HPLC, 5 cm chiralpak AD column, 60 ml/min, eluting with 100% EtOH provided Ex. 65a (Isomer a) (53 mg, 45.8%, retention time of 18.43 minutes) as a colorless foam m/z=509.3 (MH⁺). HRMS (ESI): m/z=509.2347 (MH⁺). Also obtained Ex. 65b (Isomer b) (51 mg, 44.1%, retention time of 23.53 minutes) as a colorless foam.

### Examples 71a and 71b

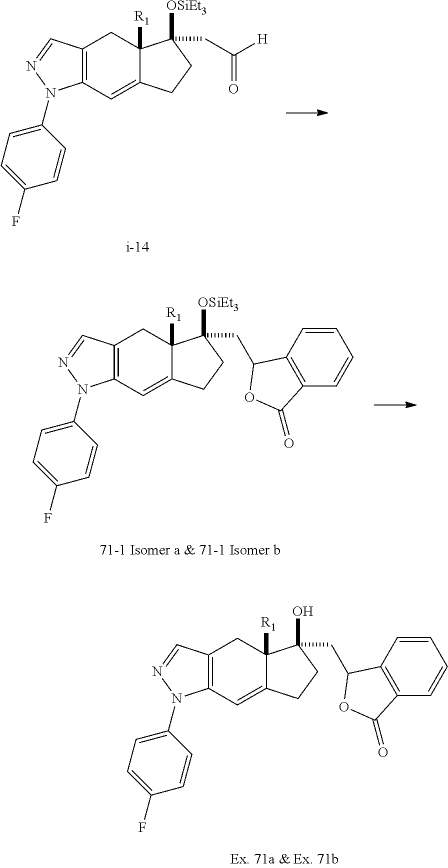

### Step A: 3-({(4aS,5R)-4a-Ethyl-1-(4-fluorophenyl)-5-[(triethylsilyl)oxy]-1,4,4a,5,6,7-hexahydrocyclopenta[f]indazol-5-yl}methyl)-2-benzofuran-1(3H)-one (71-1 Isomer a and 71-1 Isomer b)

A 2.0 M solution of isopropylmagnesium chloride (173 μl, 0.346 mmol) in tetrahydrofuran was added slowly to a solution of methyl-2-Iodobenzoate (86 mg, 0.330 mmol) in Et₂O (1650 μl) at −10° C. (ice bath=sat. NaCl/ice). Monitored formation of grignard by low mass LCMS. After about 30 min, added solution of i-14 (150 mg, 0.330 mmol) in 300 μL of THF. Reaction was allowed to warm to room temperature. Quenched reaction with sat. ammonium chloride (50 mL), extracted with ethyl acetate (25 mL, 3×). The combined organic fractions were dried over magnesium sulfate and filtered. Concentrated in vacuo. The residue was purified by column chromatography on silica gel, eluted with 30% ethyl acetate in hexanes to give 71-1 isomer a and 71-1 isomer b (50 mg, 27%, an inseparable mixture of diastereoisomers) as an orange oil. MS (ESI): m/z=559.3 (MH⁺).

### Step B: 3-{[(4aS,5R)-4a-ethyl-1-(4-fluorophenyl)-5-hydroxy-1,4,4a,5,6,7-hexahydrocyclopenta[f]indazol-5-yl]methyl}-2-benzofuran-1(3H)-one (Ex. 71a and Ex. 71b)

A 2.0 M solution of Tetra-n-butylammoniumfluoride (179 μl, 0.179 mmol) was added to a stirring solution of 71-1 (50 mg, 0.089 mmol) (mixture of diastereoisomers isomer and an isomer b) in dichloromethane (179 μL) at room temperature left stirring overnight. Upon completion the reaction mixture was concentrated in vacuo. Took up in dichloromethane, poured into brine, extracted with dichloromethane (25 mL, 3×). The combined organic fractions were dried over magnesium sulfate and filtered. Concentrated in vacuo. Diastereoisomers were purified and separated by column chromatography on silica gel, eluted with 40% ethyl acetate in hexanes to give Ex. 71a (Isomer a) (15 mg, 38%, first to elute) and Ex. 71b (Isomer b) (7 mg, 18%, second to elute) as off-white solid.

### Example 81

### (4aS,5R)-4a-Ethyl-5-{2-[3-(isopropylsulfonyl)pyridin-2-yl]ethyl}-1-phenyl-1,4,4a,5,6,7-hexahydrocyclopenta[f]indazol-5-ol (Ex. 81)

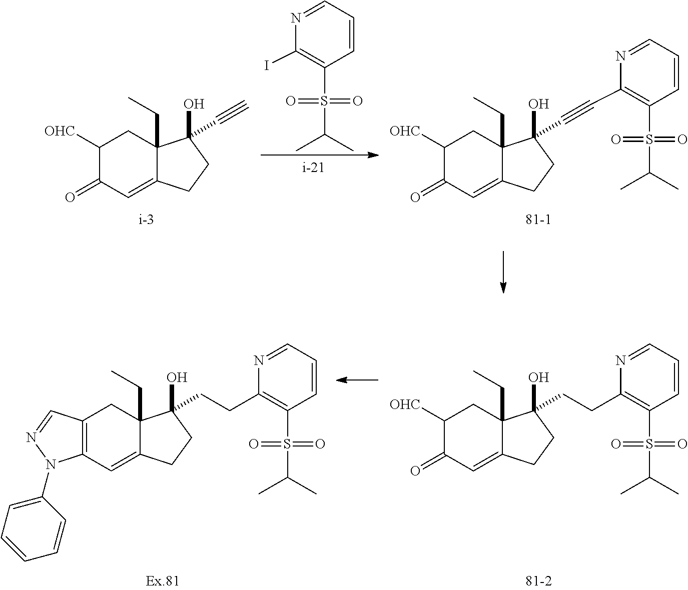

### Step A: (3R,3aS)-3a-Ethyl-3-hydroxy-3-{[3-(methylsulfonyl)pyridin-2-yl]ethynyl}-6-oxo-2,3,3a,4,5,6-hexahydro-1H-indene-5-carbaldehyde (81-1)

(3R,3aS)-3a-Ethyl-3-ethynyl-3-hydroxy-6-oxo-2,3,3a,4,5,6-hexahydro-1H-indene-5-carbaldehyde (i-3) (310 mg, 1.335 mmol), 2-iodo-3-(isopropylsulfonyl)pyridine (i-21) (457 mg, 1.468 mmol) and diisopropylamine (0.190 ml, 1.335 mmol) were dissolved in THF (1 ml) and were degassed by bubbling nitrogen through the solution for 10 minutes. Then copper (I) iodide (5.08 mg, 0.027 mmol) and bis(triphenylphosphine)palladium(II) chloride (18.74 mg, 0.027 mmol) were added and the mixture was stirred thermally at 75° C. for 2 hours. LC/MS shows the reaction was complete. Diluted mixture with ether and filtered off solid through a pad of celite and then evaporated off solvent with reduced pressure. The residue was purified by column chromatography on silica gel eluting 0-80% (7:2:1) {CHCl₃:EtOAc:MeOH) in CHCl₃ to give 81-1 (260 mg, 34%) as a brown foam. MS (ESI): m/z=416.12 (MH⁺).

### Step B: (3R,3aS)-3a-Ethyl-3-hydroxy-3-{2-[3-(isopropylsulfonyl)pyridin-2-yl]ethyl}-6-oxo-2,3,3a,4,5,6-hexahydro-1H-indene-5-carbaldehyde (81-2)

Carefully added palladium on carbon (10% Pd) (200 mg, 0.188 mmol) to a solution of (3R,3aS)-3a-ethyl-3-hydroxy-3-{[3-(methylsulfonyl)pyridin-2-yl]ethynyl}-6-oxo-2,3,3a,4,5,6-hexahydro-1H-indene-5-carbaldehyde (20-1) in EtOH (8 ml). After attaching a hydrogen balloon the flask was evacuated and backfilled with hydrogen several times. Stirred mildly at 35° C. and the reaction was complete after 3 hours. Filtered off catalyst and washed with EtOH (50 mL). Evaporated off solvent under reduced pressure and purified the residue by column chromatography on silica gel, eluting 0-70% (7:2:1) {CHCl₃:EtOAc:MeOH) in CHCl₃ to give 81-2 (135 mg, 36%) as a yellow solid. MS (ESI): m/z=420.10 (MH⁺).

### Step C: (4aS,5R)-4a-Ethyl-5-{2-[3-(isopropylsulfonyl)pyridin-2-yl]ethyl}-1-phenyl-1,4,4a,5,6,7-hexahydrocyclopenta[f]indazol-5-ol (Ex. 81)

This reaction was performed in a manner similar to that involved in making Intermediate i-4.

### Examples 82a and 82b

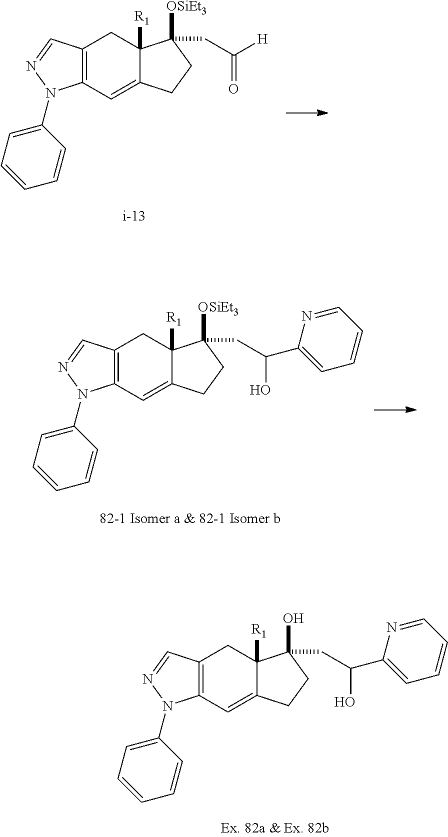

### Step A: 2-{(4aS,5R)-4a-Ethyl-1-phenyl-5-[(triethysilyl)oxy]-1,4,4a,5,6,7-hexahydrocyclopenta[f]indazol-5-yl}-1-(pyridin-2-yl)ethanol (82-1 IsomerA and 82-1 IsomerB)

n-Butyllithium (1008 μl, 2.52 mmol) was added under nitrogen to a solution of 2-bromopyridine (283 μl, 2.86 mmol) in Et₂O (2863 μl)/THF (2863 μl) at −78° C. The mixture was stirred for 20 min, followed by addition of i-13 (500 mg, 1.145 mmol) in THF (1 mL). The reaction was warmed to room temp, quenched with ammonium chloride, extracted with ethyl acetate, dried over magnesium sulfate, filtered, and concentrated in vacuo. Diastereoisomers were purified and separated by column chromatography on silica gel, eluted with 75% ethyl acetate in hexanes to give 82-1 Isomer a (200 mg, 34%, first to elute) and 82-1 Isomer b (200 mg, 34%, second to elute) as clear colorless oils. MS (ESI): m/z=516.5 (MH⁺).

### Step B: (4aS,5R)-4a-Ethyl-5-[2-hydroxy-2-(pyridin-2-yl)ethyl]-1-phenyl-1,4,4a,5,6,7-hexahydrocyclopenta[f]indazol-5-ol (Ex. 82 Isomer a or Ex. 82 Isomer b)

A 2.0 M solution of Tetra-n-butylammoniumfluoride (776 μl, 0.776 mmol) in tetrahydrofuran was added to a stirring solution of 82-1 Isomer a or 82-1 Isomer b (200 mg, 0.388 mmol) in dichloromethane at room temperature left stirring for 2 h. Upon completion the reaction mixture was concentrated in vacuo. Took up in dichloromethane, poured into brine, extracted with dichloromethane (25 mL, 3×). The combined organic fractions were dried over magnesium sulfate and filtered. Concentrated in vacuo. The residue was purified by column chromatography on silica gel, eluting with 0 to 4% methanol in dichloromethane to give Ex. 82a (Isomer a) (100 mg, 64%) or Ex. 82b (Isomer a) (110 mg, 71%) as a white crystalline solid. MS (ESI): m/z=402.4 (MH⁺).

Compounds in Table 1 were prepared following the general synthetic schemes and procedures as exemplified in the Examples described above.

TABLE 1
Chemical names and structures of Examples.

| Ex. # | STRUCTURE | NAME | HRMS |
|---|---|---|---|
| 1 | 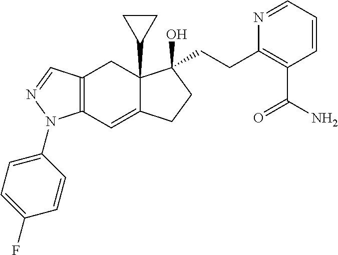 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}pyridine-3-carboxamide | 459.2189 |
| 2 | 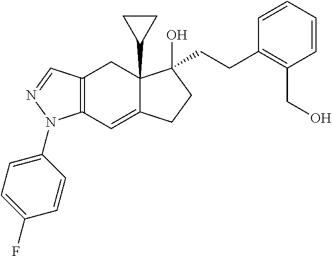 | (4aR,5R)-4a-Cyclopropyl-1-(4- fluorophenyl)-5-{2-[2- (hydroxymethyl)phenyl]ethyl}- 1,4,4a,5,6,7- hexahydrocyclopenta[f]indazol-5-ol | 445.2285 |
| 3a | 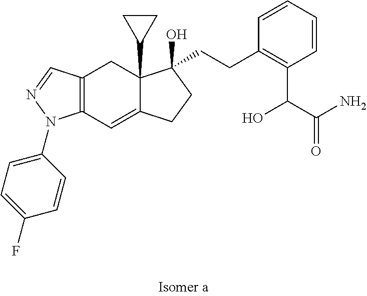 | 2-(2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}phenyl)-2-hydroxyacetamide | 488.2348 |
| 3b | 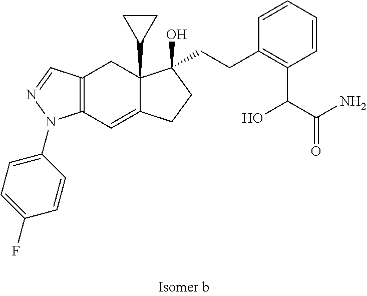 | 2-(2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}phenyl)-2-hydroxyacetamide | 488.2348 |
| 4 | 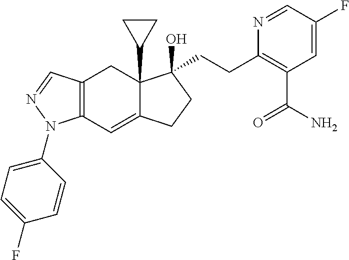 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-5-fluoropyridine-3- carboxamide | 477.2094 |
| 5 | 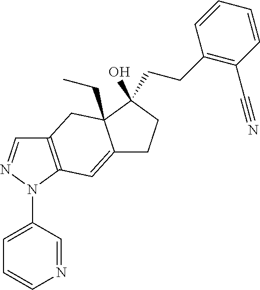 | 2-{2-[(4aS,5R)-4a-Ethyl-5-hydroxy-1- (pyridin-3-yl)-1,4,4a,5,6,7- hexahydrocyclopenta[f]indazol-5- yl]ethyl}benzonitrile | 411.2186 |
| 6 | 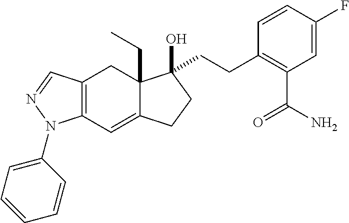 | 2-(2-(4aS,5R)-4a-Ethyl-5-hydroxy-1- phenyl-1,4,4a,5,6,7- hexahydrocyclopenta[f]indazol-5- yl}ethyl}-5-fluorobenzamide | 446.2241 |
| 7 | 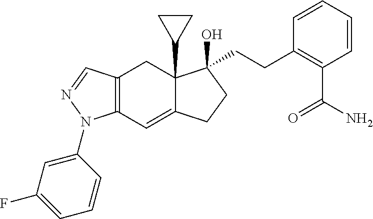 | 2-{2-[(4aR,5R)-4a-Cyclopropyl-1-(3- fluorophenyl)-5-hydroxy-1,4,4a,5,6,7- hexahydrocyclopenta[f]indazol-5- yl]ethyl}benzamide | 458.2245 |
| 8 | 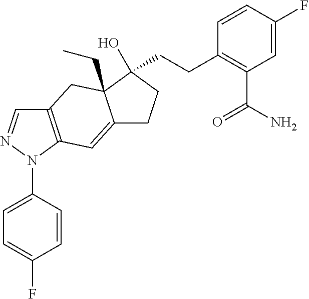 | 2-{2-[(4aS,5R)-4a-Ethyl-1-(4- fluorophenyl)-5-hydroxy-1,4,4a,5,6,7- hexahydrocyclopenta[f]indazol-5- yl]ethyl}-5-fluorobenzamide | 464.2146 |
| 9 | 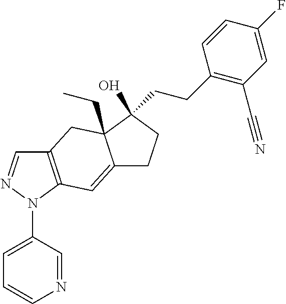 | 2-{2-[(4aS,5R)-4a-Ethyl-5-hydroxy-1- (pyridin-3-yl)-1,4,4a,5,6,7- hexahydrocyclopenta[f]indazol-5- yl]ethyl}-5-fluorobenzonitrile | 429.2087 |
| 10 | 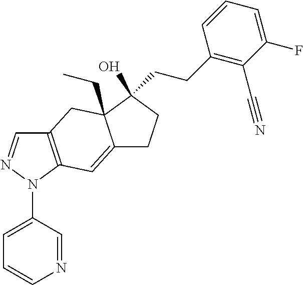 | 2-{2-[(4aS,5R)-4a-Ethyl-5-hydroxy-1- (pyridin-3-yl)-1,4,4a,5,6,7- hexahydrocyclopenta[f]indazol-5- yl]ethyl}-6-fluorobenzonitrile | 429.2090 |
| 11 | 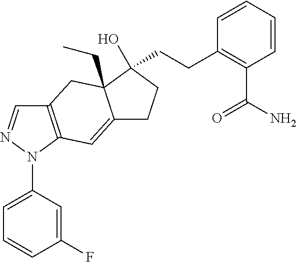 | 2-{2-[(4aS,5R)-4a-Ethyl-1-(3- fluorophenyl)-5-hydroxy-1,4,4a,5,6,7- hexahydrocyclopenta[f]indazol-5- yl]ethyl}benzamide | 446.2240 |
| 12 | 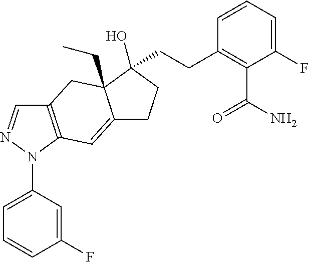 | 2-{2-[(4aS,5R)-4a-Ethyl-1-(3- fluorophenyl)-5-hydroxy-1,4,4a,5,6,7- hexahydrocyclopenta[f]indazol-5- yl]ethyl}-6-fluorobenzamide | 464.2147 |
| 13 | 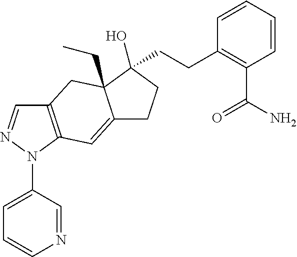 | 2-{2-[(4aS,5R)-4a-Ethyl-5-hydroxy-1- (pyridin-3-yl)-1,4,4a,5,6,7- hexahydrocyclopenta[f]indazol-5- yl]ethyl}benzamide | 429.2289 |
| 14 | 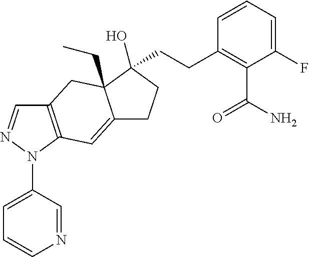 | 2-{2-[(4aS,5R)-4a-Ethyl-5-hydroxy-1- (pyridin-3-yl)-1,4,4a,5,6,7- hexahydrocyclopenta[f]indazol-5- yl]ethyl}-6-fluorobenzamide | 447.2191 |
| 15 | 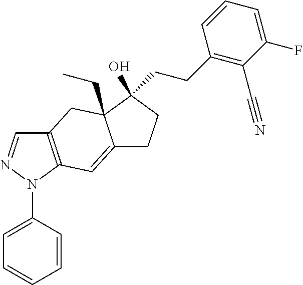 | 2-{2-[(4aS,5R)-4a-Ethyl-5-hydroxy-1- phenyl-1,4,4a,5,6,7- hexahydrocyclopenta[f]indazol-5- yl]ethyl}-6-fluorobenzonitrile | 428.2139 |
| 16 | 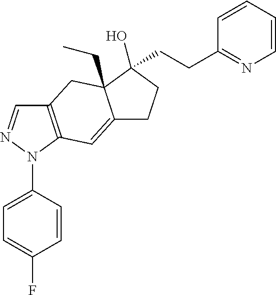 | (4aS,5R)-4a-Ethyl-1-(4-fluorophenyl)-5- [2-(pyridin-2-yl)ethyl]-1,4,4a,5,6,7- hexahydrocyclopenta[f]indazol-5-ol | 404.2144 |
| 17 | 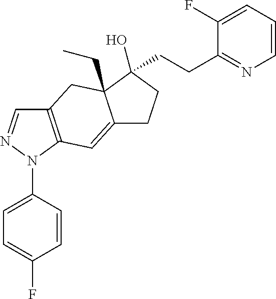 | (4aS,5R)-4a-Ethyl-1-(4-fluorophenyl)-5- [2-(3-fluoropyridin-2-yl)ethyl]- 1,4,4a,5,6,7- hexahydrocyclopenta[f]indazol-5-ol | 422.2046 |
| 18 | 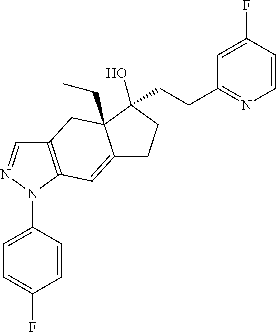 | (4aS,5R)-4a-Ethyl-1-(4-fluorophenyl)-5- [2-(4-fluoropyridin-2-yl)ethyl]- 1,4,4a,5,6,7- hexahydrocyclopenta[f]indazol-5-ol | 422.2045 |
| 19 | 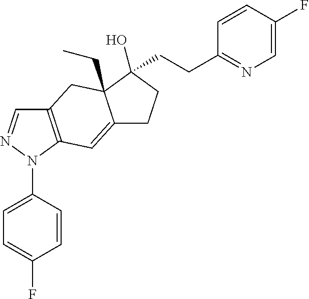 | (4aS,5R)-4a-Ethyl-1-(4-fluorophenyl)-5- [2-(5-fluoropyridin-2-yl)ethyl]- 1,4,4a,5,6,7- hexahydrocyclopenta[f]indazol-5-ol | 422.2045 |
| 20 | 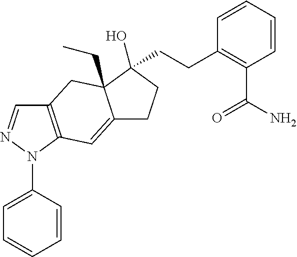 | 2-{2-[(4aS,5R)-4a-Ethyl-5-hydroxy-1- phenyl-1,4,4a,5,6,7- hexahydrocyclopenta[f]indazol-5- yl]ethyl}benzamide | 428.2337 |
| 21 | 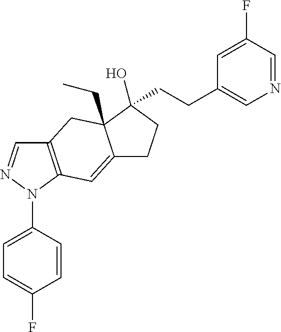 | (4aS,5R)-4a-Ethyl-1-(4-fluorophenyl)-5- [2-(5-fluoropyridin-3-yl)ethyl]- 1,4,4a,5,6,7- hexahydrocyclopenta[f]indazol-5-ol | 422.2045 |
| 22 | 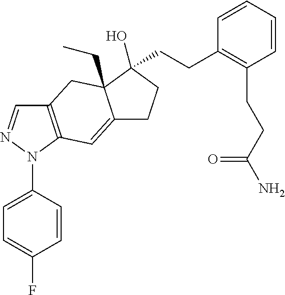 | 3-(2-{2-[(4aS,5R)-4a-Ethyl-1-(4- fluorophenyl)-5-hydroxy-1,4,4a,5,6,7- hexahydrocyclopenta[f]indazol-5- yl]ethyl}phenyl)propanamide | 474.2550 |
| 23 | 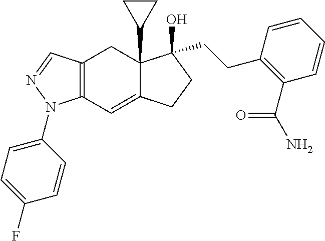 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}benzamide | 458.2238 |
| 24 | 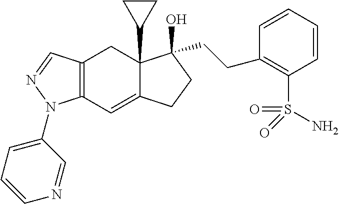 | 2-{2-[(4aR,5R)-4a-Cyclopropyl-5- hydroxy-1-(pyridin-3-yl)-1,4,4a,5,6,7- hexahydrocyclopenta[f]indazol-5- yl]ethyl}benzenesulfonamide | 477.1954 |
| 25 | 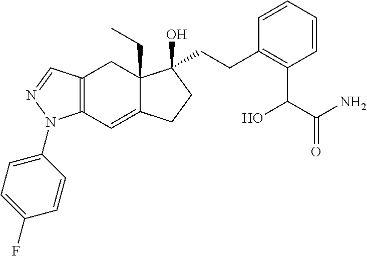 | 2-(2-{2-[(4aS,5R)-4a-Ethyl-1-(4- fluorophenyl)-5-hydroxy-1,4,4a,5,6,7- hexahydrocyclopenta[f]indazol-5- yl]ethyl}phenyl)-2-hydroxyacetamide | 476.2346 |
| 26 | 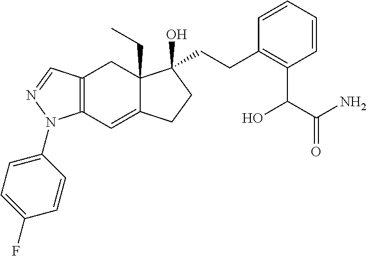 | 2-(2-{2-[(4aS,5R)-4a-Ethyl-1-(4- fluorophenyl)-5-hydroxy-1,4,4a,5,6,7- hexahydrocyclopenta[f]indazol-5- yl]ethyl}phenyl)-2-hydroxyacetamide | 476.2345 |
| 27 | 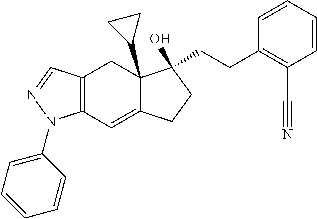 | 2-{2-[(4aR,5R)-4a-Cyclopropyl-5- hydroxy-1-phenyl-1,4,4a,5,6,7- hexahydrocyclopenta[f]indazol-5- yl]ethyl}benzonitrile | 422.2231 |
| 28 | 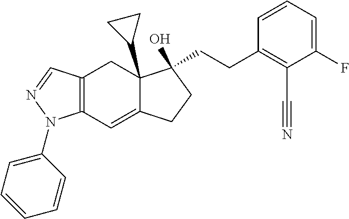 | 2-{2-[(4aR,5R)-4a-Cyclopropyl-5- hydroxy-1-phenyl-1,4,4a,5,6,7- hexahydrocyclopenta[f]indazol-5- yl]ethyl}-6-fluorobenzonitrile | 440.2137 |
| 29 | 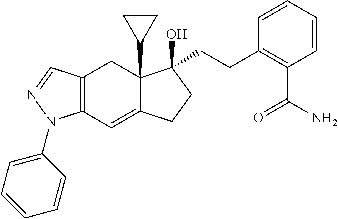 | 2-{2-[(4aR,5R)-4a-Cyclopropyl-5- hydroxy-1-phenyl-1,4,4a,5,6,7- hexahydrocyclopenta[f]indazol-5- yl]ethyl}benzamide | 440.2137 |
| 30 | 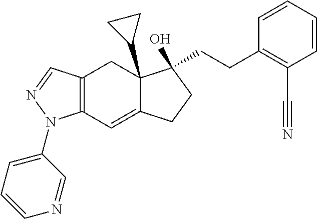 | 2-{2-[(4aR,5R)-4a-Cyclopropyl-5- hydroxy-1-(pyridin-3-yl)-1,4,4a,5,6,7- hexahydrocyclopenta[f]indazol-5- yl]ethyl}benzonitrile | 423.2183 |
| 31 | 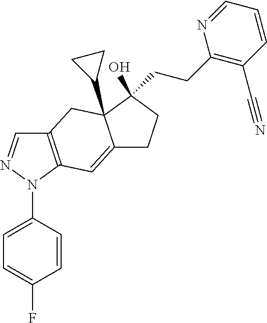 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}pyridine-3-carbonitrile | 441.2087 |
| 32 | 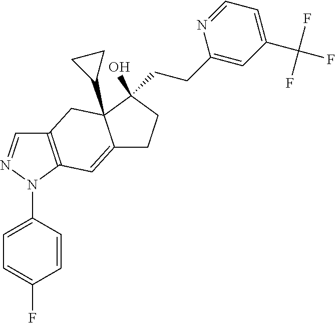 | (4aR,5R)-4a-Cyclopropyl-1-(4- fluorophenyl)-5-{2-[4- (trifluoromethyl)pyridin-2-yl]ethyl}- 1,4,4a,5,6,7- hexahydrocyclopenta[f]indazol-5-ol | 484.2007 |
| 33 | 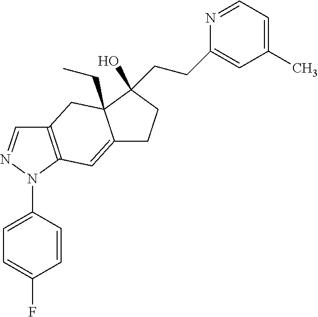 | (4aS,5R)-4a-Ethyl-1-(4-fluorophenyl)-5- [2-(4-methylpyridin-2-yl)ethyl]- 1,4,4a,5,6,7- hexahydrocyclopenta[f]indazol-5-ol | 418.2285 |
| 34 | 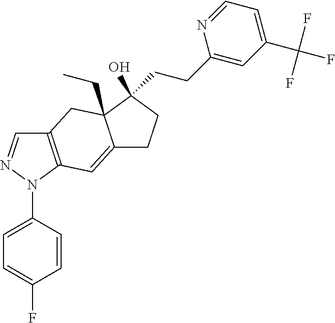 | (4aS,5R)-4a-Ethyl-1-(4-fluorophenyl)-5- {2-[4-(trifluoromethyl)pyridin-2-yl]ethyl}- 1,4,4a,5,6,7- hexahydrocyclopenta[f]indazol-5-ol | 472.2004 |
| 35 | 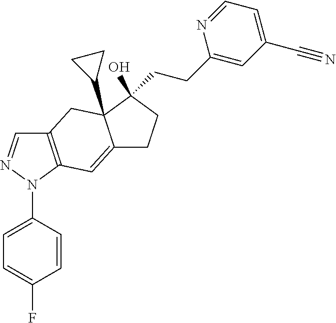 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}pyridine-4-carbonitrile | 441.2088 |
| 36 | 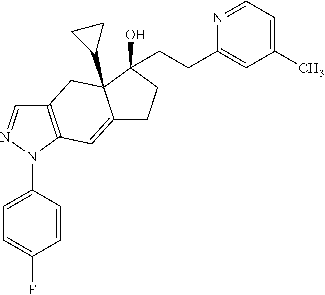 | (4aR,5R)-4a-Cyclopropyl-1-(4- fluorophenyl)-5-[2-(4-methylpyridin-2- yl)ethyl]-1,4,4a,5,6,7- hexahydrocyclopenta[f]indazol-5-ol | 430.2289 |
| 37 | 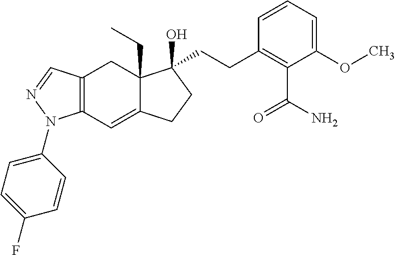 | 2-{2-[(4aS,5R)-4a-Ethyl-1-(4- fluorophenyl)-5-hydroxy-1,4,4a,5,6,7- hexahydrocyclopenta[f]indazol-5- yl]ethyl}-6-methoxybenzamide | 476.2339 |
| 38 | 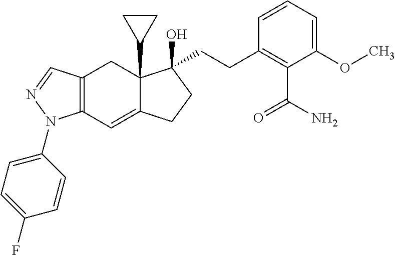 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-6-methoxybenzamide | 488.2341 |
| 39 | 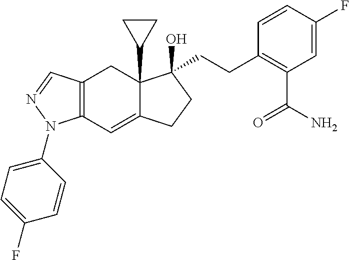 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-5-fluorobenzamide | 476.2133 |
| 40 | 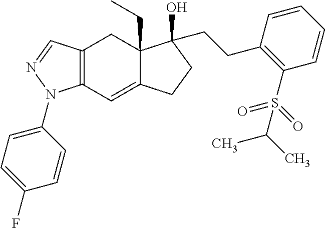 | (4aS,5R)-4a-Ethyl-1-(4-fluorophenyl)-5- {2-[2-(propan-2- ylsulfonyl)phenyl]ethyl}-1,4,4a,5,6,7- hexahydrocyclopenta[f]indazol-5-ol | 509.2280 |
| 41 | 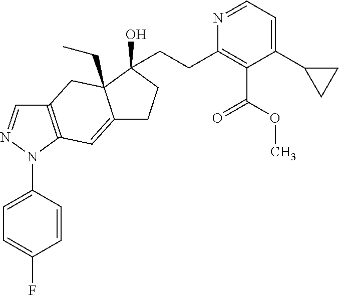 | Methyl 4-cyclopropyl-2-{2-[(4aS,5R)-4a- ethyl-1-(4-fluorophenyl)-5-hydroxy- 1,4,4a,5,6,7- hexahydrocyclopenta[f]indazol-5- yl]ethyl}pyridine-3-carboxylate | 502.2502 |
| 42 | 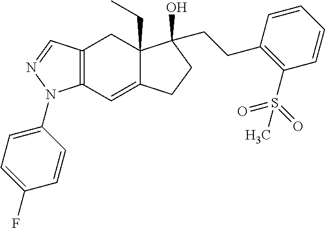 | (4aS,5R)-4a-Ethyl-1-(4-fluorophenyl)-5- {2-[2-(methylsulfonyl)phenyl]ethyl}- 1,4,4a,5,6,7- hexahydrocyclopenta[f]indazol-5-ol | 481.1971 |
| 43 | 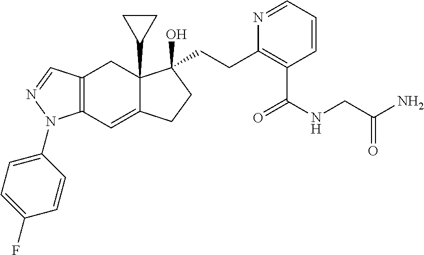 | N-(2-Amino-2-oxoethyl)-2-{2-[(4aR,5R)- 4a-cyclopropyl-1-(4-fluorophenyl)-5- hydroxy-1,4,4a,5,6,7- hexahydrocyclopenta[f]indazol-5- yl]ethyl}pyridine-3-carboxamide | 516.2405 |
| 44 | 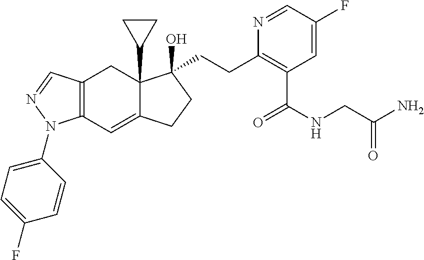 | N-(2-Amino-2-oxoethyl)-2-{2-[(4aR,5R)- 4a-cyclopropyl-1-(4-fluorophenyl)-5- hydroxy-1,4,4a,5,6,7- hexahydrocyclopenta[f]indazol-5- yl]ethyl}-5-fluoropyridine-3- carboxamide | 534.2316 |
| 45 | 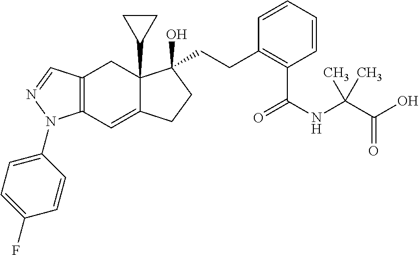 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-N-(1-hydroxy-2-methylpropan- 2-yl)benzamide | 530.2825 |
| 46 | 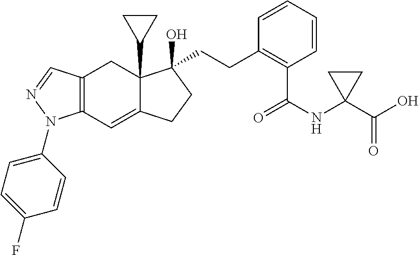 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-N-[1- (hydroxymethyl)cyclopropyl]benzamide | 528.2662 |
| 47 | 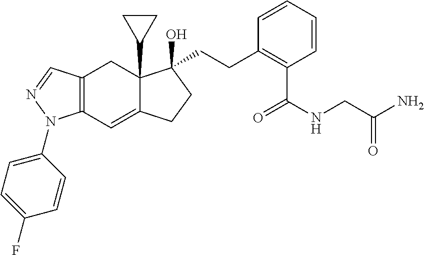 | N-(2-Amino-2-oxoethyl)-2-{2-[(4aR,5R)- 4a-cyclopropyl-1-(4-fluorophenyl)-5- hydroxy-1,4,4a,5,6,7- hexahydrocyclopenta[f]indazol-5- yl]ethyl}benzamide | 515.2460 |
| 48 | 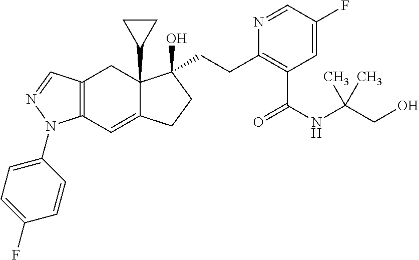 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-5-fluoro-N-(1-hydroxy-2- methylpropan-2-yl)pyridine-3- carboxamide | 549.2671 |
| 49 | 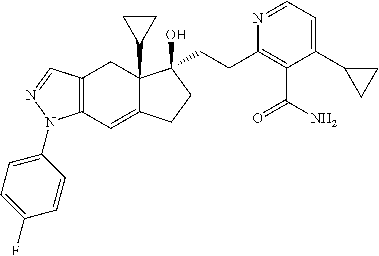 | 4-Cyclopropyl-2-{2-[(4aR,5R)-4a- cyclopropyl-1-(4-fluorophenyl)-5- hydroxy-1,4,4a,5,6,7- hexahydrocyclopenta[f]indazol-5- yl]ethyl}pyridine-3-carboxamide | 499.2506 |
| 50 | 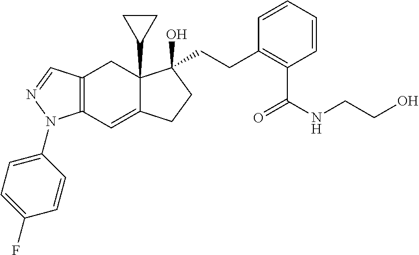 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-N-(2-hydroxyethyl)benzamide | 502.2507 |
| 51 | 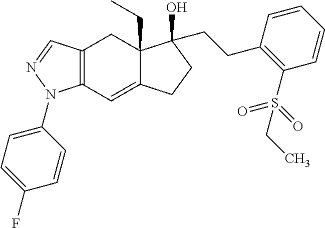 | (4aS,5R)-4a-Ethyl-5-{2-[2- (ethylsulfonyl)phenyl]ethyl}-1-(4- fluorophenyl)-1,4,4a,5,6,7- hexahydrocyclopenta[f]indazol-5-ol | 495.2124 |
| 52 | 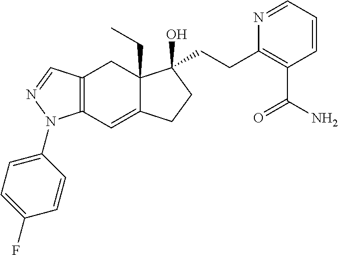 | 2-{2-[(4aS,5R)-4a-Ethyl-1-(4- fluorophenyl)-5-hydroxy-1,4,4a,5,6,7- hexahydrocyclopenta[f]indazol-5- yl]ethyl}pyridine-3-carboxamide | 447.2191 |
| 53 | 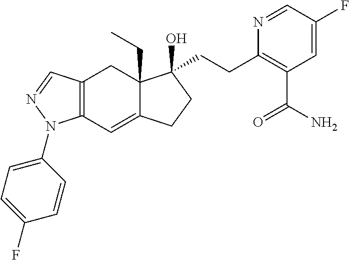 | 2-{2-[(4aS,5R)-4a-Ethyl-1-(4- fluorophenyl)-5-hydroxy-1,4,4a,5,6,7- hexahydrocyclopenta[f]indazol-5- yl]ethyl}-5-fluoropyridine-3- carboxamide | 465.2095 |
| 54 | 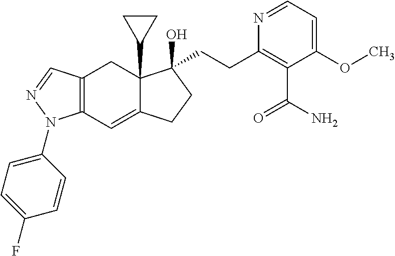 | 2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-4-methoxypyridine-3- carboxamide | 489.2298 |
| 55 | 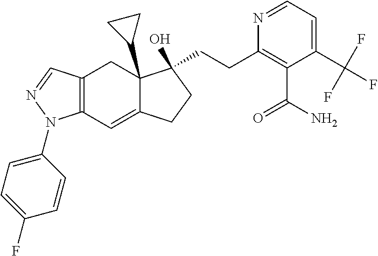 | 2-{2-[(4aR,5R)-4a-Cyclopropyl-1-(4- fluorophenyl)-5-hydroxy-1,4,4a,5,6,7- hexahydrocyclopenta[f]indaozol-5- yl]ethyl}-4-(trifluoromethyl)pyridine-3- carboxamide | 527.2058 |
| 56 | 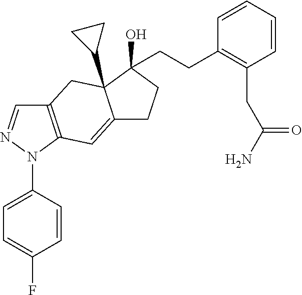 | 2-(2-{2-[(4aR,5S)-4a-Cyclopropyl-1-(4- fluorophenyl)-5-hydroxy-1,4,4a,5,6,7- hexahydrocyclopenta[f]indazol-5- yl]ethyl}phenyl)acetamide | 472.2386 |
| 57 | 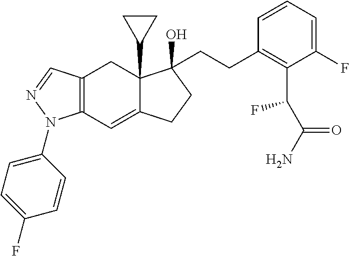 | (2R)-2-(2-{2-[(4aR,5R)-4a-Cyclopropyl- 1-(4-fluorophenyl)-5-hydroxy- 1,4,4a,5,6,7- hexahydrocyclopenta[f]indazol-5- yl]ethyl}-6-fluorophenyl)-2- fluoroethanamide | 508.2194 |
| 58 | 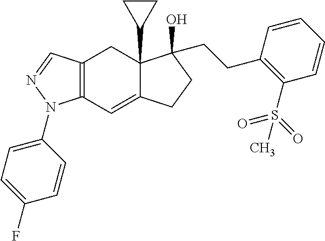 | (4aR,5R)-4a-Cyclopropyl-1-(4- fluorophenyl)-5-{2-[2- (methylsulfonyl)phenyl]ethyl}- 1,4,4a,5,6,7- hexahydrocyclopenta[f]indazol-5-ol | 493.1968 |
| 59 | 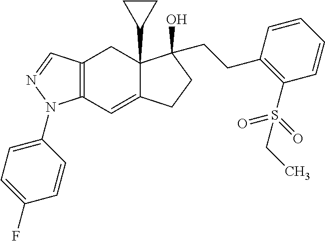 | (4aR,5R)-4a-Cyclopropyl-5-{2-[2- (ethylsulfonyl)phenyl]ethyl}-1-(4- fluorophenyl)-1,4,4a,5,6,7- hexahydrocyclopenta[f]indazol-5-ol | 507.2126 |
| 60 | 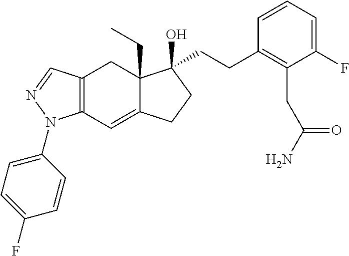 | 2-(2-{2-[(4aS,5R)-4a-Ethyl-1-(4- fluorophenyl)-5-hydroxy-1,4,4a,5,6,7- hexahydrocyclopenta[f]indazol-5- yl]ethyl}-6-fluorophenyl)acetamide | 478.2293 |
| 61 | 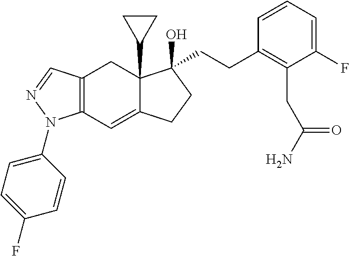 | 2-(2-{2-[(4aR,5R)-4a-Cyclopropyl-1-(4- fluorophenyl)-5-hydroxy-1,4,4a,5,6,7- hexahydrocyclopenta[f]indazol-5- yl]ethyl}-6-fluorophenyl)acetamide | 490.2291 |
| 62 | 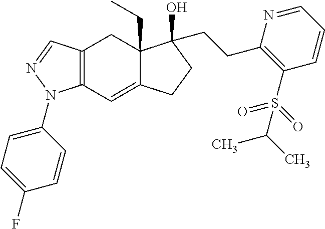 | (4aS,5R)-4a-Ethyl-1-(4-fluorophenyl)-5- {2-[3-(propan-2-ylsulfonyl)pyridin-2- yl]ethyl}-1,4,4a,5,6,7- hexahydrocyclopenta[f]indazol-5-ol | 510.2234 |
| 63 | 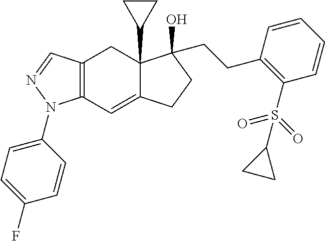 | (4aR,5R)-4a-Cyclopropyl-5-{2-[2- (cyclopropylsulfonyl)phenyl]ethyl}-1-(4- fluorophenyl)-1,4,4a,5,6,7- hexahydrocyclopenta[f]indazol-5-ol | 519.2126 |
| 64 | 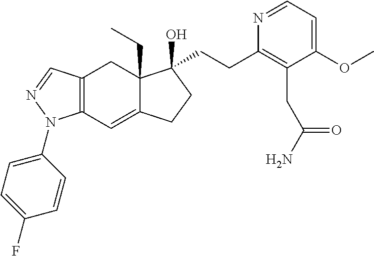 | 2-(2-{2-[(4aS,5R)-4a-Ethyl-1-(4- fluorophenyl)-5-hydroxy-1,4,4a,5,6,7- hexahydrocyclopenta[f]indazol-5- yl]ethyl}-4-methoxypyridin-3- yl)acetamide | 491.2440 |
| 65 | 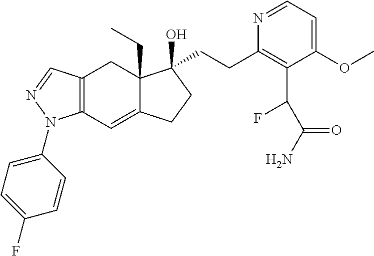 | 2-(2-{2-[(4aS,5R)-4a-Ethyl-1-(4- fluorophenyl)-5-hydroxy-1,4,4a,5,6,7- hexahydrocyclopenta[f]indazol-5- yl]ethyl}-4-methoxypyridin-3-yl)-2- fluoroacetamide | 509.2347 |
| 66 | 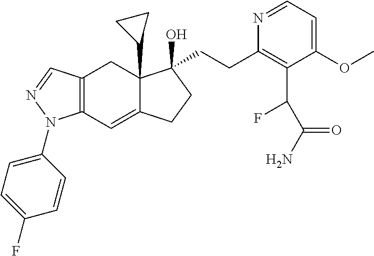 | 2-(2-{2-[(4aR,5R)-4a-Cyclopropyl-1- (4-fluorophenyl)-5-hydroxy- 1,4,4a,5,6,7- hexahydrocyclopenta[f]indazol-5- yl]ethyl}-4-methoxypyridin-3-yl)-2- fluoroacetamide | 521.2350 |
| 67a | 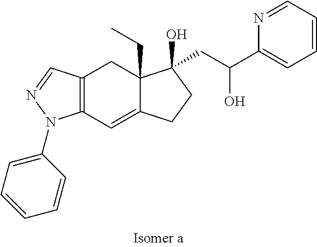 | (4aS,5R)-4a-Ethyl-5-[2-hydroxy-2- (pyridin-2-yl)ethyl]-1-phenyl- 1,4,4a,5,6,7- hexahydrocyclopenta[f]indazol-5-ol | 402.2183 |
| 67b | 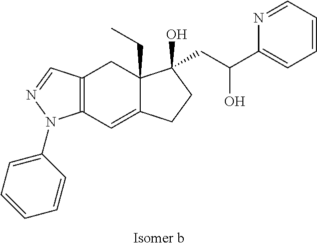 | (4aS,5R)-4a-Ethyl-5-[2-hydroxy-2- (pyridin-2-yl)ethyl]-1-phenyl- 1,4,4a,5,6,7- hexahydrocyclopenta[f]indazol-5-ol | 402.2174 |
| 68 | 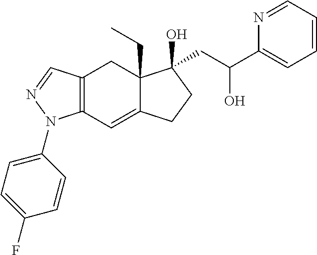 | (4aS,5R)-4a-Ethyl-1-(4-fluorophenyl)- 5-[2-hydroxy-2-(pyridin-2-yl)ethyl]- 1,4,4a,5,6,7- hexahydrocyclopenta[f]indazol-5-ol | 420.2085 |
| 69a | 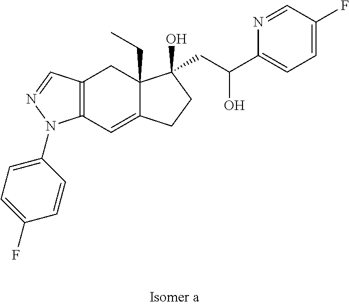 | (4aS,5R)-4a-Ethyl-1-(4-fluorophenyl)- 5-[2-(5-fluoropyridin-2-yl)-2- hydroxyethyl]-1,4,4a,5,6,7- hexahydrocyclopenta[f]indazol-5-ol | 438.3 (MS ESI) |
| 69b | 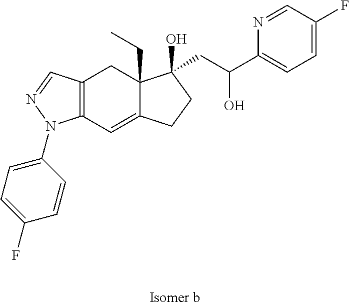 | (4aS,5R)-4a-Ethyl-1-(4-fluorophenyl)- 5-[2-(5-fluoropyridin-2-yl)-2- hydroxyethyl]-1,4,4a,5,6,7- hexahydrocyclopenta[f]indazol-5-ol | 438.4 (MS ESI) |
| 70 | 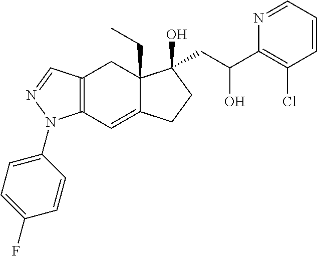 | (4aS,5R)-5-[2-(3-Chloropyridin-2-yl)- 2-hydroxyethyl]-4a-ethyl-1-(4- fluorophenyl)-1,4,4a,5,6,7- hexahydrocyclopenta[f]indazol-5-ol | 454.1694 |
| 71 | 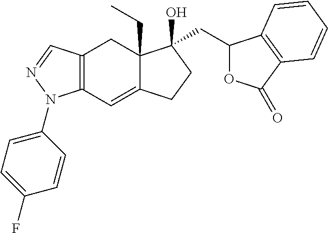 | 3-{[(4aS,5R)-4a-Ethyl-1-(4- fluorophenyl)-5-hydroxy-1,4,4a,5,6,7- hexahydrocyclopenta[f]indazol-5- yl]methyl}-2-benzofuran-1(3H)-one | 445.2 (MS ESI) |
| 72 | 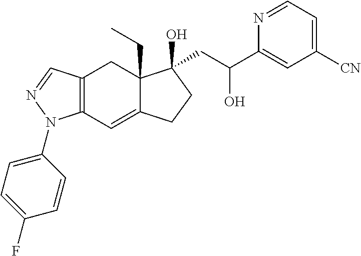 | 2-{2-[(4aS,5R)-4a-Ethyl-1-(4- fluorophenyl)-5-hydroxy-1,4,4a,5,6,7- hexahydrocyclopenta[f]indazol-5-yl]- 1-hydroxyethyl}pyridine-4-carbonitrile | 445.2026 |
| 73 | 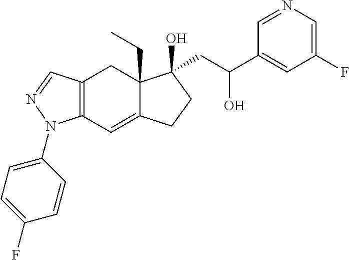 | (4aS,5R)-4a-Ethyl-1-(4-fluorophenyl)- 5-[2-(5-fluoropyridin-3-yl)-2- hydroxyethyl]-1,4,4a,5,6,7- hexahydrocyclopenta[f]indazol-5-ol | 438.1991 |
| 74 | 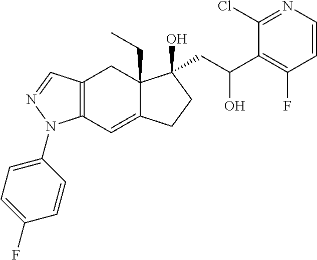 | (4aS,5R)-5-[2-(2-Chloro-4- fluoropyridin-3-yl)-2-hydroxyethyl]- 4a-ethyl-1-(4-fluorophenyl)- 1,4,4a,5,6,7- hexahydrocyclopenta[f]indazol-5-ol | 472.1601 |
| 75 | 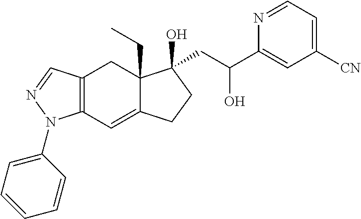 | 2-{2-[(4aS,5R)-4a-Ethyl-5-hydroxy-1- phenyl-1,4,4a,5,6,7- hexahydrocyclopenta[f]indazol-5-yl]- 1-hydroxyethyl}pyridine-4-carbonitrile | 427.2131 |
| 76 | 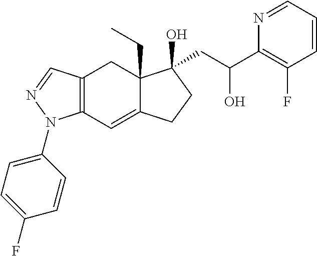 | (4aS,5R)-4a-Ethyl-1-(4-fluorophenyl)- 5-[2-(3-fluoropyridin-2-yl)-2- hydroxyethyl]-1,4,4a,5,6,7- hexahydrocyclopenta[f]indazol-5-ol | 438.1983 |
| 77 | 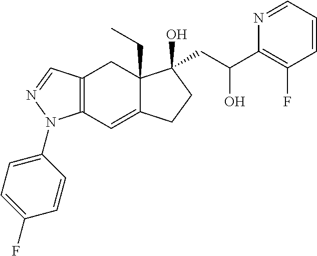 | (4aS,5R)-4a-Ethyl-1-(4-fluorophenyl)- 5-[2-(3-fluoropyridin-2-yl)-2- hydroxyethyl]-1,4,4a,5,6,7- hexahydrocyclopenta[f]indazol-5-ol | 438.1985 |
| 78 | 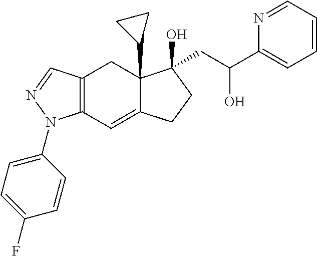 | (4aR,5R)-4a-Cyclopropyl-1-(4- fluorophenyl)-5-[2-hydroxy-2- (pyridin-2-yl)ethyl]-1,4,4a,5,6,7- hexahydrocyclopenta[f]indazol-5-ol | 432.2085 |
| 79a | 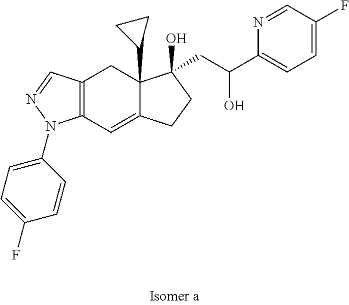 | (4aR,5R)-4a-Cyclopropyl-1-(4- fluorophenyl)-5-[2-(5-fluoropyridin-2- yl)-2-hydroxyethyl]-1,4,4a,5,6,7- hexahydrocyclopenta[f]indazol-5-ol | 450.1985 |
| 79b | 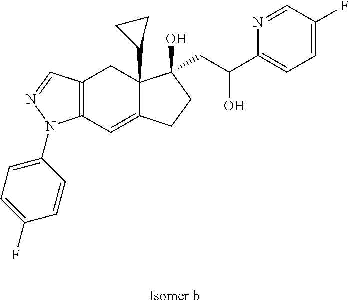 | (4aR,5R)-4a-Cyclopropyl-1-(4- fluorophenyl)-5-[2-(5-fluoropyridin-2- yl)-2-hydroxyethyl]-1,4,4a,5,6,7- hexahydrocyclopenta[f]indazol-5-ol | 450.1986 |
| 80 | 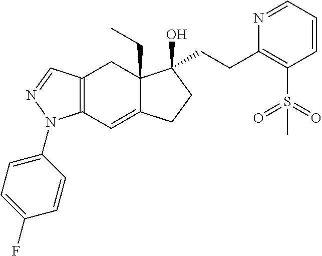 | (4aS,5R)-4a-Ethyl-1-(4-fluorophenyl)- 5-{2-[3-(methylsulfonyl)pyridin-2- yl]ethyl}-1,4,4a,5,6,7- hexahydrocyclopenta[f]indazol-5-ol | 482.1910 |
| 81 | 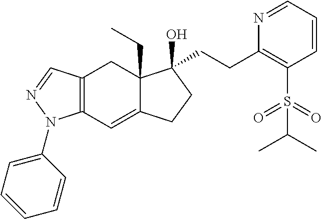 | (4aS,5R)-4a-Ethyl-5-{2-[3-(iso- propylsulfonyl)pyridin-2- yl]ethyl}-1-phenyl-1,4,4a,5,6,7- hexahydrocyclopenta[f]indazol-5-ol | 492.2320 |
| 82a | 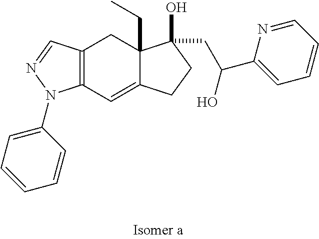 | (4aS,5R)-4a-Ethyl-5-[2-hydroxy-2- (pyridin-2-yl)ethyl]-1-phenyl- 1,4,4a,5,6,7- hexahydrocyclopenta[f]indazol-5-ol | 402.2183 |
| 82b | 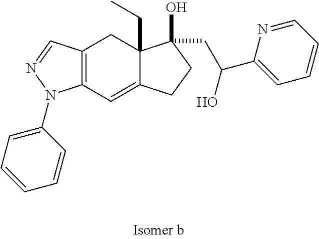 | (4aS,5R)-4a-Ethyl-5-[2-hydroxy-2- (pyridin-2-yl)ethyl]-1-phenyl- 1,4,4a,5,6,7- hexahydrocyclopenta[f]indazol-5-ol | 402.2174 |

### Biological Evaluation

The compounds exemplified above exhibited activity in one or more of the following assays.

### Ligand Binding Assay

### Materials:

Binding Buffer: TEGM (10 mM Tris-HCl, 1 mM EDTA, 10% glycerol, 1 mM betamecaptoethanol, 10 mM Sodium Molybdate, pH 7.2)

### 50% HAP Slurry: Calbiochem Hydroxylapatite, Fast Flow, in 10 mM Tris, pH 8.0 and 1 mM EDTA.

Wash Buffer: 40 mM Tris, pH7.5, 100 mM KCl, 1 mM EDTA and 1 mM EGTA. 95% EtOH Dexmethasone-methyl-3H, (DEXA*); (Amersham cat#TRK645)
Dexamethasone(DEX) (Sigma, cat#01756):

### Hydroxylapatite Fast Flow; Calbiochem Cat#391947

### 25 Molybdate=Molybdic Acid (Sigma, M1651)

### HeLa Cell Culture Media:

RPMI 1640 (Gibco 11835-055) w/23.8 mM NaHC03, 2 mM L-glutamine in 500 mL of complete media Final cone.
30 10 mL (1M Hepes) 20 mM
5 mL (200 mM L-glu) 4 mM
0.5 mL (10 mg/mL human insulin) 10 Ilg/mL in 0.01 N HCl Calbiochem#407694-S)
50 mL FBS (Sigma F2442) 10%
1 mL (10 mglmL Gentamicin 20 μg/mL Gibco#15710-072)

### Cell Passaging

5 Cells (Hall R. E., et al., European Journal of Cancer, 30A: 484-490 (1994)) HeLa (ATCC) cultured in RPMI 1640 (Gibed 11835-055) containing 20 mM Hepes, 4 mM L-glu, 10 ug/ml of human insulin (Sigma, 1-0259), 10% FBS and 20 uglml of Gentamicin (Gibco#15710072) are rinsed twice in PBS. Phenol red-free Trypsin-EDTA is diluted in the same PBS 1:10. The cell layers are rinsed with IX Trypsin, extra Trypsin is poured out, and the cell layers are incubated at 37° C. for 2 min. The flask is tapped and checked for signs of cell detachment. Once the cells begin to slide off the flask, the complete media is added. The cells are counted at this point, then diluted to the appropriate concentration and split into flasks or dishes for further culturing (Usually 1:3 to 1:6 dilution).

### Preparation of HeLa Cell Lysate

When the cells are 70 to 85% confluent, they are detached as described above, and collected by centrifuging at 1000 g for 10 min at 4° C. The cell pellet is washed twice with TEGM (10 mM Tris-HCl, 1 mM EDTA, 10% glycerol, 1 mM beta-mercaptoethanol, 10 mM Sodium Molybdate, pH 7.2). After the final wash, the cells are resuspended in TEGM at a concentration of 107 cells/mL. The cell suspension is snap frozen in liquid nitrogen or ethanol/dry ice bath and transferred to −80° C. freezer on dry ice. Before setting up the binding assay, the frozen samples are left on ice-water to just thaw. Then the samples are centrifuged at 12,500 g to 20,000 g for 30 min at 4° C. The supernatant is used to set-up assay right away. If using 50 μL of supernatant, the test compound can be prepared in 50 μL of the TEGM buffer.

### Procedure for Multiple Compound Screening

1×TEGM buffer is prepared, and the isotope-containing assay mixture is prepared in the following order: EtOH (2% final concentration in reaction), 3H-DEX (Amersham Biosciences) and 1×TEGM. [e.g. For 100 samples, 200 JlL (100×2) of EtOH+4.25 JlL of 1:10 ³H-Dex stock+2300 μL (100×23) 1×TEGM]. The compound is serially diluted, e.g., if starting final conc. is 1 μM, and the compound is in 25 μL of solution, for duplicate samples, 75 μL of 4×1 μM solution is made and 3 μL of 100). 1M is added to 72 μL of buffer, and 1:5 serial dilution.

25 μL of ³H-DEX (6 nM) trace and 25 μL, compound solution are first mixed together, followed by addition of 50 μL receptor solution. The reaction is gently mixed, spun briefly at about 200 rpm and incubated at 4° C. overnight. 100 μL of 50% HAP slurry is prepared and added to the incubated reaction which is then vortexed and incubated on ice for 5 to 10 min. The reaction mixture is vortexed twice more to resuspend HAP while incubating reaction. The samples in 96-well format are then washed in wash buffer using the FilterMate™ Universal Harvester plate washer (Packard). The washing process transfers HAP pellet containing ligand-bound expressed receptor to Unifilter-96 GFIB filter plate (Packard). The HAP pellet on the filter plate is incubated with 50 mL of MICROSCINT (Packard) scintillant for 30 min before being counted on the TopCount microscintillation counter (Packard). IC50s are calculated using DEX as a reference.

### Trans-Activation Modulation of Glucocorticoid Receptor (GRAMMER)

This assay assesses the ability of test compounds to control transcription from the MMTV-LUC reporter gene in lung adenocarcinoma A549 cells or HeLa cells, a human breast cancer cell line that naturally expresses the human GR. The assay measures induction of a modified MMTV LTR/promoter linked to the LUC reporter gene.

The routine transient assay consists of plating 7,000-25,000 cells/well of a white, clear-bottom 96-well plate. Alternatively, 384-well plates can be used at a cell concentration of 10,000/well. The media that the cells are plated in is “exponential growth medium” which consists of phenol red-free RPMI1640 containing 10% FBS, 4 mM L-glutamine, 20 mM HEPES, 10 ug/mL human insulin, and 20 ug/mL gentamicin. Incubator conditions are 37° C. and 5% C0₂. The transfection is done in batch mode. The cells are trypsinized and counted to the right cell number in the proper amount of fresh media. It is then gently mixed with the FuGene6/DNA 25 mix and plated onto the 96 or 384-well plate, all the wells receive 100 uL or 40 uL, respectively, of medium+lipid/DNA complex then incubated 37° C. overnight. The transfection cocktail consists of serum-free OptiMEM, FuGene6 reagent and DNA. The manufacturer's (Roche Biochemical) protocol for cocktail setup is as follows: The lipid to DNA ratio is approximately 2.5:1 and the incubation time is 20 min at room temperature. Sixteen to 24 h after transfection, the cells are treated with dexamethasone to a final concentration of 10 nM as well as the compound of interest, such that final DMSO (vehicle) concentration is equal to or less than 1%. Each plate also contains samples that are treated with 10 nM dexamethasone alone, which is used as the 100% activity control. The cells are exposed to the compounds for 24 h. After 24 h, the cells are lysed by a Promega cell culture lysis buffer for approximately 30 min and then the luciferase activity in the extracts is assayed in the 96-well format luminometer. In 384 well format, Steady-Glo (Promega) or Steady-Lite (PerkinElmer) can be used by adding an equal volume of reagent to the media present in each well. Activity induced by 10 nM dexamethasone alone is set at 100% activity. Antagonist activity is calculated by determining the decrease in dexamethasone-induced activity in response to compound treatment relative to samples that were treated with dexamethasone alone. Results are expressed as % inhibition of 10 nM dexamethasone activity or as fold of 10 nM dexamethasone activity. This transactivation assay can be performed in an agonist and antagonist mode to identify these different activities.

Activity of test compounds is calculated as the Emax relative to the activity obtained with 300 nM dexamethasone. Activity of test compounds is calculated as the Emax relative to the activity obtained with 300 nM DEX. The exemplified tissue selective glucocorticoid receptor modulators of the present invention display agonist activity in this assay of greater than 5% and less than 100%, and maximal transactivation activity less then maximal transrepression activity. The action of compounds is also tested in an antagonist mode (Anti-GRAMMER) in which the cells are treated with medium containing an agonist such as 10 nM DEX and the ability of agents to inhibit the activation by an agonist is measured.

### Transrepression Assay (GITAR)

This assay assesses the ability of test compounds to control transcription from the TNFα-β-lactamase reporter gene in U937 cells, a human myelomonocytic leukemia cell line that naturally expresses the human GR. The assay measures compound dependent-repression of the TNFa promoter linked to a reporter gene.

The human U937 cells that had been stablely transfected with the TNF-a promoter driving ˜-lactamase are used for this assay. U937 cells contain an endogenous glucocorticoid receptor (GR). Cells are maintained in RPMI 1640 Growth medium (GibcoCat#11875-093) containing 25 mM HEPES, 10% FBS, 2 mM L-Glutamine, 1 mM Sodium pyruvate, 25 μg/ml Gentamicin (Gibed Cat#1571 0-064), 1:1000 2-Mercaptoethanol (Gibco Cat#21985-023) and 0.8 mg/ml G418 (Gibco Cat#10131-027). The density of the cells in the flask needs to be about 1×10⁶-3×10⁶/ml at the time of harvest. Usually, the cells are split to 1.2˜1.4×105/ml (1:10) 3 days prior to the assay. 50,000 cells/well are plated in 96 well black-walled plates the day of assay. Test compounds are added 10 J.lL/well, and cells are incubated at 37° C. for 30˜45 min. For assaying compounds, first dilute 1:10 in DMSO to make 1 mM, then further dilute 1:100 in medium to make 10× stock prior to adding to the cells. Add 50 ng/ml PMA (Sigma, cat#P8139) 10 μL/well to a final concentration 5 ng/ml, and 1 μg/ml LPS (Sigma, cat#L4130) 10 μL/well to a final concentration 100 ng/ml. Incubate cells at 370 C overnight for −18 hr. PMA is stored frozen as 10 μg/ml stock in DMSO. Dilute 1:10 in DMSO for a working stock of 10 μg/ml and store at −20° C. For assaying, dilute the 10 μg/ml working stock 1:200 in medium to make a 10× solution (50 ng/ml). Store frozen LPS at 1 mg/ml in PBS, dilute 1:1000 in medium to make 10×μg/ml) for the assay. Add 6× loading buffer (CCF2-AM) 20 μL/well, and incubate at room temperature for 70-90 min. Read plates on CytoFluor II Plate Reader according to manufacture suggested protocols. The activity repressed by 100 nM dexamethasone alone is set as 100% activity.

### Microarray Analysis

This assay assesses the ability of test compounds to modulate the transcription of endogenously expressed genes in a variety of cell types including but not limited to A549, HeLa or U937 cells. All cell culture reagents were purchased from Invitrogen Life Tech, Carlsbad Calif. A549 cells were grown in phenol red-free DMEM1F12 medium supplemented with 10% PBS. Cells were grown at 370 C with 5% C02. Using the RNeasy Kit (Qiagen Corp, Valencia Calif.), total RNA was extracted and purified from A549 cells treated with different GC compounds for 24 h, at a fully active dose. These cells express large amount of the GR and are very responsive to GC treatment. All samples were compared against cells treated with vehicle. Expression levels of 23000 genes were measured using oligonucleotide microarrays purchased from Agilent Technologies, Inc. Each comparison was done on a pair of microarrays with reversed fluorophores. Raw image intensity data were processed according to the method described in U.S. Pat. No. 6,351,712. The method was used to remove dye bias and to derive a Rosetta probability (P) and fold change value for each gene and each sample pair. Furthermore, for each gene an ANOVA model was constructed across all treatments to derive error estimates. P values for evaluating expression differences were computed using a Bayesian adjusted t-test that was developed by Lonnstedt and Speed (2002) and extended by Smyth (2003). A gene was declared differentially expressed in any particular comparison if it satisfied two criteria:

1. The Rosetta p value had to be less than 0.1 and the Rosetta fold change value 30 had to be greater than 1.4 in at least one of the treatments.

2. The ANOVA p value had to be less than 0.01 and the fold change greater than 2 in the comparison under consideration.

### In Vivo Inflammation Assay

Intact adult (6 month old) female Sprague-Dawley rats are used in the oxazolone (OX) contact dermatitis model. Rats were sensitized on the ventral abdomen with OX on Day 0. On Days 7 and 9, a randomly-selected ear was challenged (same ear each time) with OX; the other was treated with vehicle. Daily treatment begun on. Day 7 and continued for 7 d with test compounds at different doses and 1.3 mpk 6-methylprednisolone or 0.1 mpk DEX as positive controls. The thickness of both ears are measured on days 11 and 14. Necropsy occurred on Day 14. The rat is first weighed, then anesthetized in a CO₂ chamber until near death. Approximately 5 ml whole blood is obtained by cardiac puncture. The rat is then examined for certain signs of death and completeness. Tissues are dissected in a highly stylized fashion. Then the following endpoints were evaluated: a) inhibiting ear inflammation induced by oxazalone, b) raising serum insulin, c) reducing serum ACTH, d) reducing spleen weight, e) reducing skin thickness, f) reducing body weight, g) increasing expression of bone-related genes with potential relationship to negative glucocorticoid effects on bone; h) changes in molecular markers that correlate with skin inflammation, skin thinning, muscle atrophy and glucose metabolism in liver. All blood samples were collected between 1330-1530 h, −4-5 hrs after the last compound treatment.

Primary data for this assay are left and right ear thickness. Inter-ear thickness difference (etd) is used for the estimating the level of inflammation and effectiveness of the compounds is determined by their ability to reduce the increase the thickness of the inflamed ear. Back of the rat skin thickness, spleen weight, serum insulin as well as the effects of gcs on the expression of molecular markers in skin inflammation, skin atrophy, muscle atrophy and glucose metabolism in liver are measured. Data are analyzed by anova plus fisher plsd post-hoc test to identify intergroup differences.

### Results

The compounds shown in Table 1 were tested in the binding, GRAMMER and GITAR assays and the results are shown in Table 2. These compounds demonstrated a superior activity profile, for example, these compounds have potencies in the GRAMMER and GITAR assays (as measured by inflection points, IP) of less than 300 nM concomitant with maximum activity in the GRAMMER assay of less than 60% and maximum activity in the GITAR assay of between 40 and 90%.

Compounds in the range of activities described above offer potential improvements over fuller agonists (higher Emaxes) as they may have less side effects as demonstrated in preclinical models. Among the compounds with the described range of activities, different selectivity profiles may be observed in different animal models, which model the side effects of diabetes and glucose intolerance, skin and muscle atrophy, intraocular pressure, bone degradation, and hypertension.

TABLE 2
Biological assay results of Examples.

| | | Transactivation | | Transrepression | |
|---|---|---|---|---|---|
| Example | GRBind | A549 Cells Grammer | | U937 Cells GITAR | |
| # | KI (nM) | IP (nM) | Emax (%) | IP (nM) | Emax (%) |
| 1 | 4.51 | 61 | 27 | 55 | 77 |
| 2 | 1.32 | 22 | 24 | 8 | 79 |
| 3a | 1.80 | 125 | 7 | 85 | 72 |
| 3b | 1.79 | 108 | 19 | 68 | 85 |
| 4 | 1.98 | 116 | 14 | 83 | 71 |
| 5 | 2.01 | 47 | 29 | 31 | 80 |
| 6 | 3.17 | 178 | 26 | 143 | 75 |
| 7 | 3.22 | 143 | 7 | 143 | 67 |
| 8 | 1.27 | 106 | 47 | 90 | 88 |
| 9 | 1.99 | 76 | 15 | 120 | 71 |
| 10 | 2.59 | 116 | 20 | 113 | 64 |
| 11 | 3.22 | 83 | 41 | 80 | 82 |
| 12 | 1.85 | 23 | 58 | 25 | 87 |
| 13 | 28.38 | 224 | 34 | 163 | 76 |
| 14 | 8.68 | 88 | 51 | 43 | 88 |
| 15 | 1.28 | 62 | 29 | 114 | 89 |
| 16 | 2.22 | 80 | 31 | 93 | 79 |
| 17 | 1.44 | 30 | 44 | 44 | 89 |
| 18 | 1.73 | 52 | 32 | 74 | 80 |
| 19 | 2.52 | 156 | 18 | 263 | 77 |
| 20 | 2.49 | 45 | 51 | 28 | 86 |
| 21 | 1.04 | 76 | 31 | 95 | 87 |
| 22 | 1.48 | 123 | 28 | 148 | 89 |
| 23 | 1.80 | 37 | 21 | 22 | 77 |
| 24 | 2.19 | 24 | 35 | 7 | 83 |
| 25 | 3.23 | 195 | 22 | 118 | 74 |
| 26 | 3.82 | 161 | 58 | 51 | 86 |
| 27 | 1.00 | 23 | 13 | 51 | 86 |
| 28 | 1.24 | 32 | 8 | 122 | 88 |
| 29 | 2.29 | 74 | 12 | 47 | 69 |
| 30 | 1.36 | 126 | 4 | 178 | 63 |
| 31 | 1.58 | 9 | 24 | 13 | 89 |
| 32 | 2.26 | 65 | 11 | 266 | 83 |
| 33 | 2.33 | 72 | 27 | 84 | 82 |
| 34 | 2.23 | 91 | 24 | 125 | 85 |
| 35 | 1.99 | 25 | 17 | 73 | 87 |
| 36 | 1.63 | 59 | 11 | 114 | 72 |
| 37 | 2.02 | 10 | 49 | 8 | 90 |
| 38 | 1.25 | 10 | 11 | 11 | 74 |
| 39 | 1.98 | 116 | 14 | 83 | 71 |
| 40 | 2.45 | 101 | 36 | 38 | 83 |
| 41 | 2.63 | 138 | 28 | 262 | 79 |
| 42 | 1.97 | 16 | 34 | 11 | 89 |
| 43 | 5.47 | 260 | 47 | 52 | 86 |
| 44 | 4.60 | 154 | 39 | 38 | 86 |
| 45 | 2.09 | 22 | 39 | 9 | 85 |
| 46 | 1.99 | 20 | 34 | 7 | 90 |
| 47 | 1.43 | 17 | 46 | 9 | 83 |
| 48 | 3.55 | 26 | 50 | 9 | 87 |
| 49 | 2.70 | 9 | 30 | 12 | 78 |
| 50 | 2.09 | 12 | 57 | 9 | 88 |
| 51 | 2.45 | 92 | 28 | 65 | 83 |
| 52 | 9.83 | 64 | 59 | 53 | 85 |
| 53 | 5.97 | 77 | 49 | 75 | 86 |
| 54 | 2.44 | 35 | 23 | 48 | 77 |
| 55 | 2.24 | 7 | 38 | 2 | 87 |
| 56 | 1.61 | 14 | 9 | 95 | 78 |
| 57 | 1.50 | 31 | 11 | 106 | 86 |
| 58 | 0.88 | 9 | 11 | 5 | 88 |
| 59 | 1.36 | 35 | 7 | 74 | 88 |
| 60 | 2.33 | 38 | 39 | 24 | 84 |
| 61 | 2.08 | 21 | 13 | 16 | 72 |
| 62 | 4.06 | 35 | 43 | 12 | 85 |
| 63 | 1.69 | 42 | 5 | 127 | 75 |
| 64 | 11.52 | 142 | 37 | 72 | 77 |
| 65 | 9.20 | 175 | 34 | 195 | 63 |
| 66 | 1.82 | 7 | 45 | 12 | 76 |
| 67a | 11.31 | 284 | 20 | 217 | 54 |
| 67b | 5.46 | 174 | 10 | 277 | 44 |
| 68 | 2.72 | 49 | 21 | 18 | 71 |
| 69a | 4.41 | 170 | 33 | 111 | 74 |
| 69b | 2.91 | 113 | 26 | 94 | 80 |
| 70 | 7.06 | 83 | 31 | 142 | 79 |
| 71 | 2.97 | 78 | 30 | 65 | 74 |
| 72 | 6.25 | 187 | 13 | 228 | 51 |
| 73 | 5.67 | 101 | 34 | 198 | 71 |
| 74 | 9.35 | 65 | 41 | 103 | 72 |
| 75 | 2.97 | 106 | 10 | 235 | 67 |
| 76 | 20.79 | 142 | 31 | 275 | 64 |
| 77 | 4.39 | 115 | 23 | 162 | 60 |
| 78 | 2.96 | 66 | 13 | 213 | 49 |
| 79a | 2.83 | 186 | 10 | 167 | 41 |
| 79b | 1.55 | 46 | 8 | 282 | 54 |
| 80 | 1.32 | 56 | 38 | 56 | 70 |
| 81 | 7.30 | 96 | 36 | 53 | 68 |
| 82a | 11.31 | 285 | 20 | 217 | 54 |
| 82b | 5.46 | 174 | 10 | 277 | 44 |

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A compound of Formula I, or a pharmaceutically acceptable salt thereof, or a stereoisomer thereof, or a pharmaceutically acceptable salt of the stereoisomer thereof: 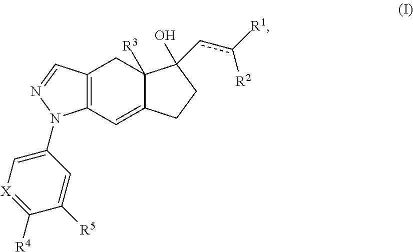
or a pharmaceutically acceptable salt thereof, or a stereoisomer thereof, or a pharmaceutically acceptable salt of the stereoisomer thereof:
wherein
“ [symbol] ” is an optional double bond;
X is —CH═ or —N═;
R¹ is selected from the group consisting of:
(1) phenyl, and
(2) HET;
wherein each of items (1) and (2) is optionally substituted with one to three groups independently selected from:
(a) halogen,
(b) —OR^{a},
(c) —C(O)—R^{a},
(d) —C(O)—OR^{a},
(e) —C(O)NR^{a}R^{b},
(f) C₁-6alkyl, optionally substituted with one to three groups selected from halogen, hydroxy, and —C(O)NR^{a}R^{b},
(g) C₃₋₆cycloalkyl,
(h) —SO₂R^{a}, and
(i) nitrile;
R² is selected from the group consisting of:
(1) hydrogen,
(2) C₁₋₆alkyl, and
(3) hydroxy;
R³ is selected from the group consisting of:
(1) ethyl,
(2) —CH₂CF₃, and
(3) cyclopropyl;
each of R⁴ and R⁵ is independently selected from the group consisting of:
(1) hydrogen, and
(2) halogen; and
each of R^{a} and R^{b} is independently selected from the group consisting of:
(1) hydrogen,
(2) C₁₋₆alkyl, optionally substituted with one to three groups selected from halogen, hydroxy, C₃₋₆cycloalkyl, and —C(O)NH₂, and
(3) C₃₋₆cycloalkyl, optionally substituted with one to three groups selected from halogen, C₁₋₆alkyl, and C₁₋₆alkoxy; and
HET is selected from the group consisting of: pyridazinyl, pyridyl, pyrimidyl, furanyl, thiazolyl, and oxazolyl.

2. The compound of claim 1, wherein
R¹ is selected from the group consisting of:
(1) phenyl, and
(2) pyridyl;
wherein each of items (1) to (2) is optionally substituted with one to three groups independently selected from:
(a) halogen,
(b) —OR^{a},
(c) —C(O)—R^{a},
(d) —C(O)—OR^{a},
(e) —C(O)NR^{a}R^{b},
(f) C₁₋₄alkyl, optionally substituted with one to three groups selected from halogen, hydroxy, and —C(O)NR^{a}R^{b},
(g) C₃₋₆cycloalkyl,
(h) —SO₂R^{a}, and
(i) nitrile.

3. The compound of claim 1, wherein
X is —CH—; and
R¹ is pyridyl, wherein the pyridyl is optionally substituted with one to three groups independently selected from:
(a) halogen,
(b)—OR^{a},
(c)—C(O)—OR^{a},
(d) —C(O)—OR^{a},
(e) —C(O)NR^{a}R^{b},
(f) C₁₋₄alkyl, optionally substituted with one to three groups selected from halogen, hydroxy, and —C(O)NR^{a}R^{b},
(g) C₃₋₆cycloalkyl,
(h) —SO₂R^{a}, and
(i) nitrile.

4. The compound of claim 1, wherein
X is —N═; and
R¹ is selected from the group consisting of
(1) phenyl, and
(2) pyridyl;
wherein each of items (1) to (2) is optionally substituted with one to three groups independently selected from:
(a) halogen,
(b) —OR^{a},
(c) —C(O)—R^{a},
(d) —C(O)—OR^{a},
(e) —C(O)NR^{a}R^{b},
(f) C₁₋₄alkyl, optionally substituted with one to three groups selected from halogen, hydroxy, and —C(O)NR^{a}R^{b},
(g) C₃₋₆cycloalkyl,
(h) —SO₂R^{a}, and
(i) nitrile.

5. The compound of claim 1, wherein
R² is hydrogen or hydroxy.

6. The compound of claim 1, wherein
R³ is selected from the group consisting of:
(1) ethyl, and
(2) cyclopropyl.

7. The compound of claim 1, wherein
each of R⁴ and R⁵ is independently selected from the group consisting of:
(1) hydrogen,
(2) chloro,
(3) fluoro, and
(4) bromo.

8. The compound of claim 1, wherein
each of R^{a} and R^{b} is independently selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from halogen, methyl, ethyl, propyl, methoxy, and ethoxy.

9. The compound of claim 1 of Formula Ia: 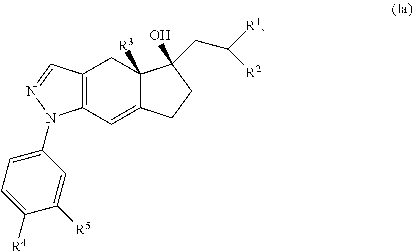
wherein
R¹ is selected from the group consisting of:
(1) phenyl, and
(2) pyridyl;
wherein each of items (1) and (2) is optionally substituted with one to three groups independently selected from:
(a) halogen,
(b) —OR^{a},
(e) —C(O)—R^{a},
(d) —C(O)—OR^{a},
(f) —C(O)NR^{a}R^{b},
(f) C₁₋₆alkyl, optionally substituted with one to three groups selected from halogen, hydroxy, and —C(O)NR^{a}R^{b},
(g) C₃₋₆cycloalkyl,
(h) —SO₂R^{a}, and
(i) nitrile;
R² is hydrogen or hydroxy;
R³ is selected from the group consisting of:
(1) ethyl, and
(2) cyclopropyl;
each of R⁴ and R⁵ is independently selected from the group consisting of:
(1) hydrogen, and
(2) halogen; and
each of R^{a} and R^{b} is independently selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from halogen, methyl, ethyl, propyl, methoxy, and ethoxy.

10. The compound of claim 9, wherein
R¹ is selected from the group consisting of:
(1) phenyl, and
(2) pyridyl;
wherein each of items (1) and (2) is optionally substituted with one to three groups independently selected from:
(a) halogen,
(b) —OR^{a},
(c)—C(O)—R^{a},
(d) —C(O)—OR^{a},
(e) —C(O)NH₂,
(f) C₁₋₄alkyl, optionally substituted with one to three groups selected from halogen, hydroxy, and —C(O)NH₂,
(g) cyclopropyl,
(h) —SO₂R^{a}, and
(i) nitrile; and
R^{a} is selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from methyl, ethyl, propyl, and methoxy.

11. The compound of claim 9, wherein
R⁴ is hydrogen or fluoro, and
R⁵ is hydrogen or fluoro.

12. The compound of claim 1 of Formula Ib: 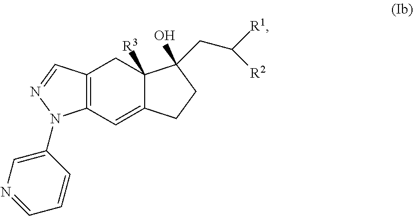
wherein
R¹ is selected from the group consisting of
(1) phenyl, and
(2) pyridyl;
wherein each of items (1) and (2) is optionally substituted with one to three groups independently selected from:
(a) halogen,
(b) —OR^{a},
(c)—C(O)—R^{a},
(d) —C(O)—OR^{a},
(e) —C(O)NR^{a}R^{b},
(f) C₁₋₆alkyl, optionally substituted with one to three groups selected from halogen, hydroxy, and —C(O)NR^{a}R^{b},
(g) C₃₋₆cycloalkyl,
(h) —SO₂R^{a}, and
(i) nitrile;
R² is hydrogen or hydroxy;
R³ is selected from the group consisting of:
(1) ethyl, and
(2) cyclopropyl; and
each of R^{a} and R^{b} is independently selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from halogen, methyl, ethyl, propyl, methoxy, and ethoxy.

13. The compound of claim 12, wherein
R¹ is selected from the group consisting of:
(1) phenyl, and
(2) pyridyl;
wherein each of items (1) and (2) is optionally substituted with one to three groups independently selected from:
(a) halogen,
(b) —OR^{a},
(c) —C(O)—R^{a},
(d) —C(O)—OR^{a},
(e) —C(O)NH₂,
(f) C₁₋₄alkyl, optionally substituted with one to three groups selected from halogen, hydroxy, and —C(O)NH₂,
(g) cyclopropyl,
(h) —SO₂R^{a}, and
(i) nitrile; and
R^{a} is selected from the group consisting of:
(1) hydrogen,
(2) C₁₋₄alkyl, optionally substituted with one to three groups selected from halogen, hydroxy, cyclopropyl, and —C(O)NH₂, and
(3) cyclopropyl, optionally substituted with one to three groups selected from methyl, ethyl, propyl, and methoxy.

14. The compound of claim 1, selected from the group consisting of:
Compound Structure

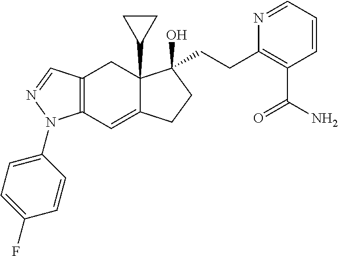

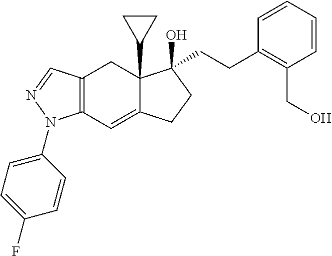

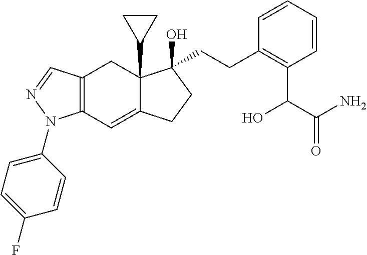

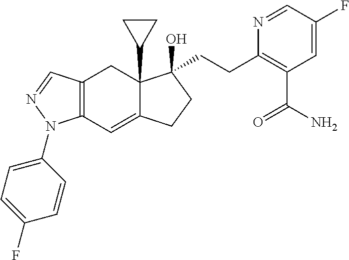

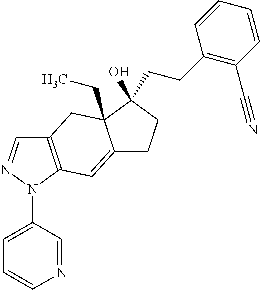

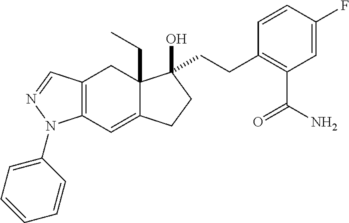

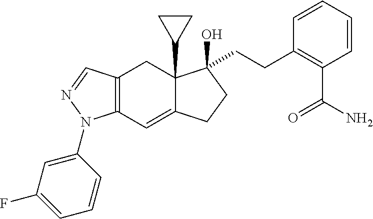

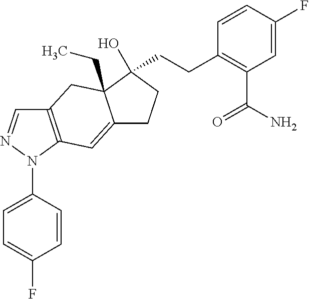

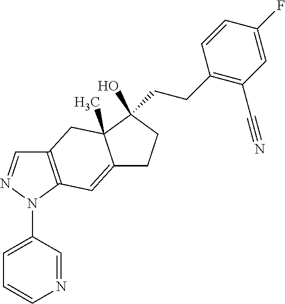

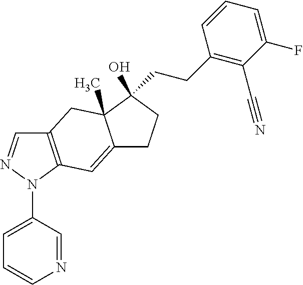

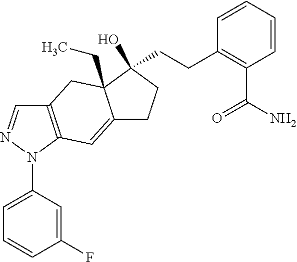

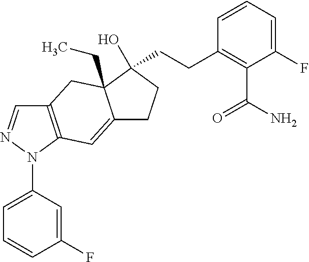

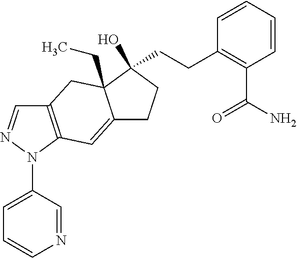

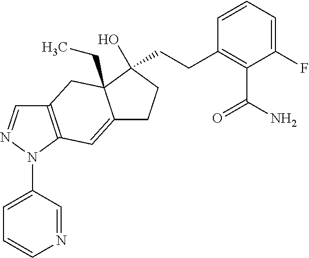

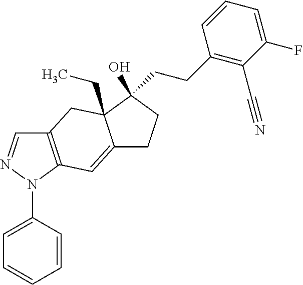

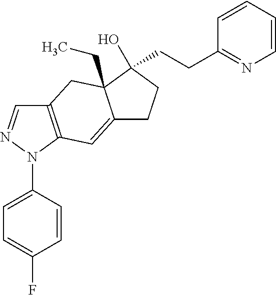

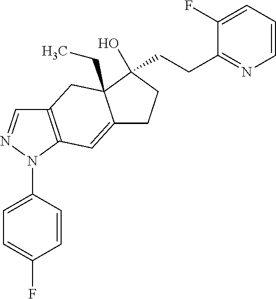

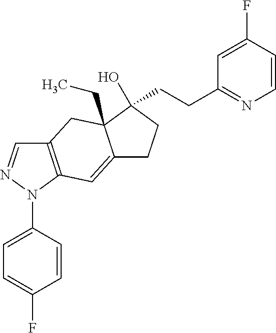

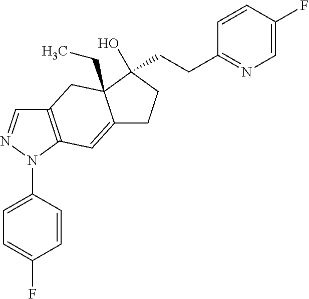

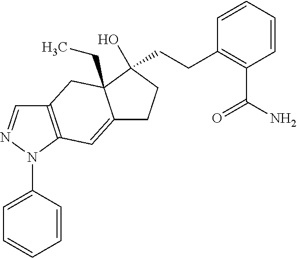

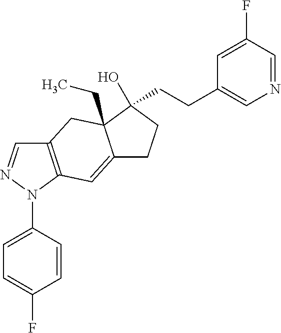

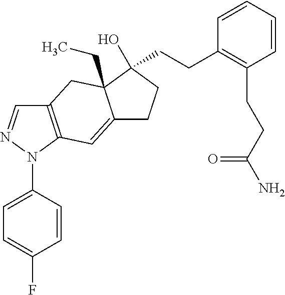

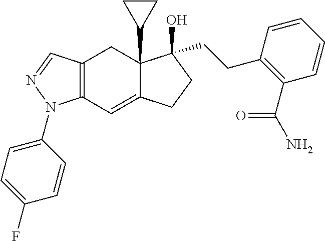

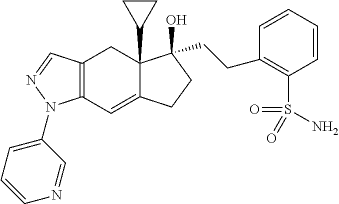

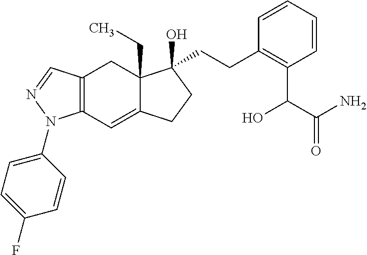

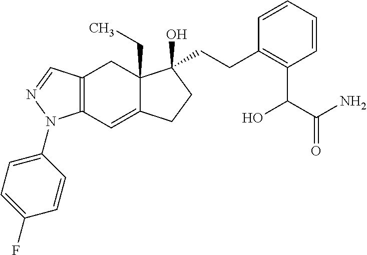

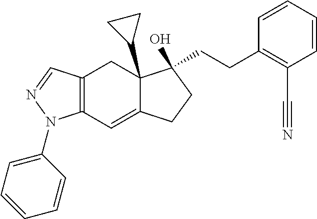

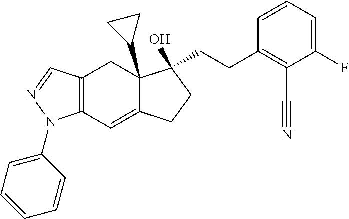

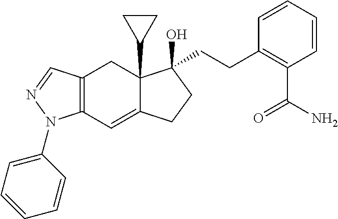

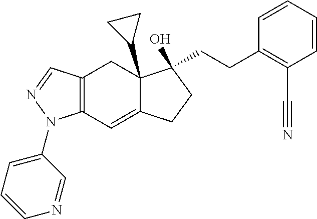

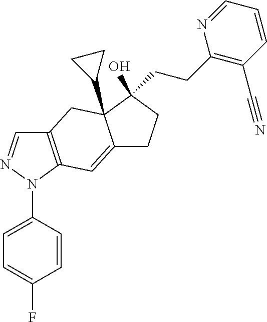

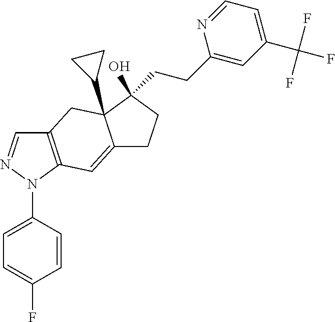

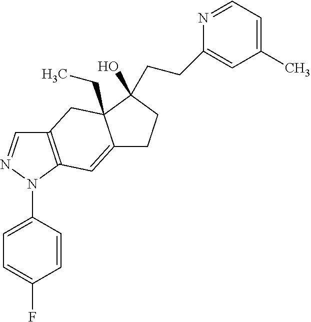

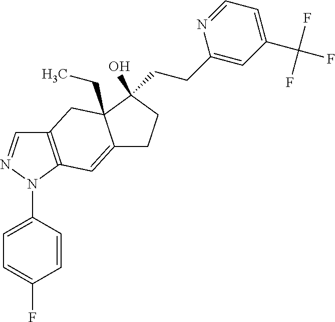

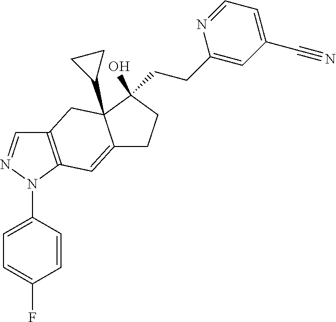

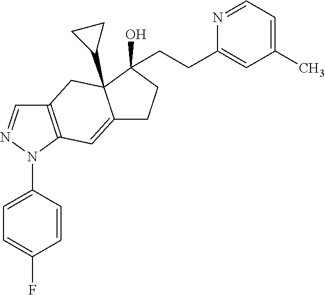

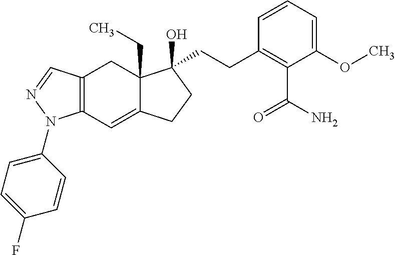

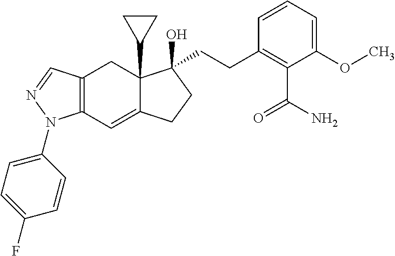

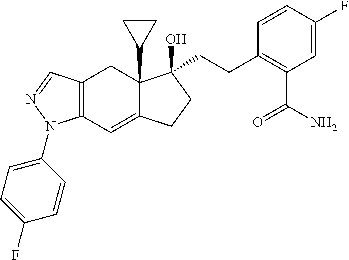

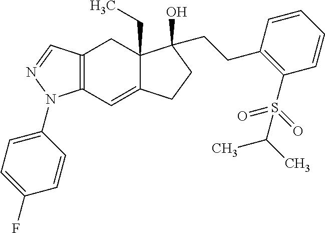

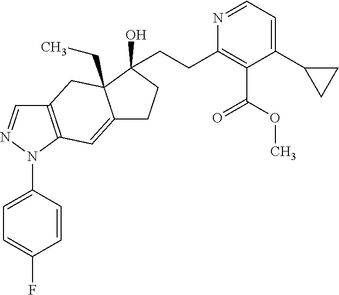

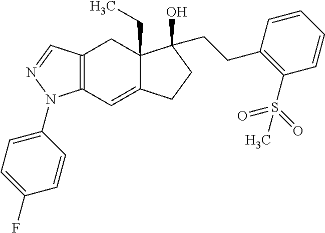

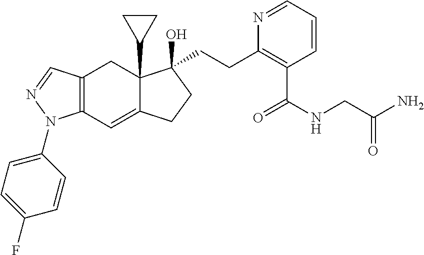

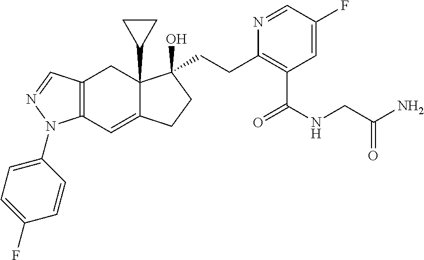

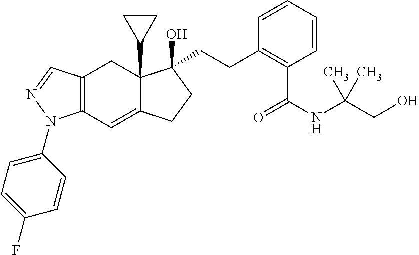

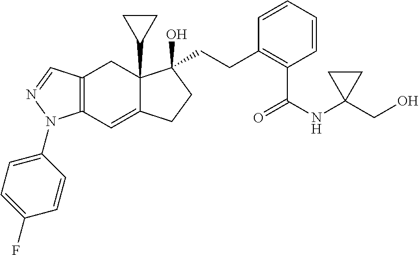

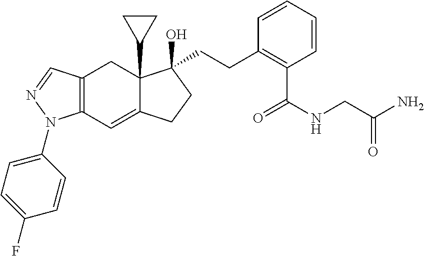

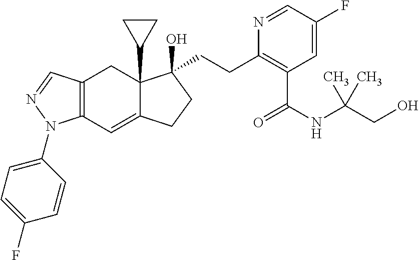

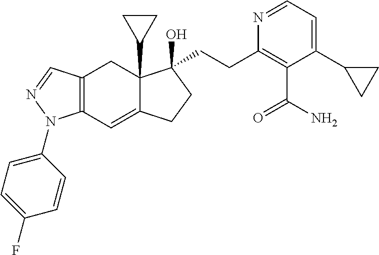

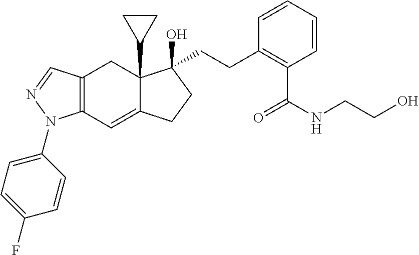

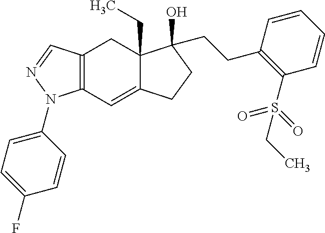

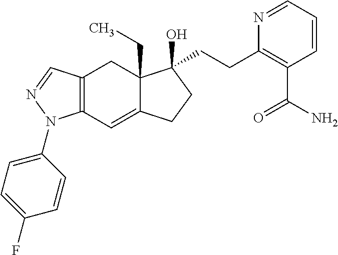

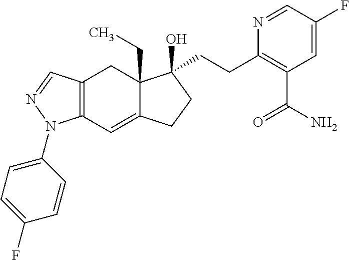

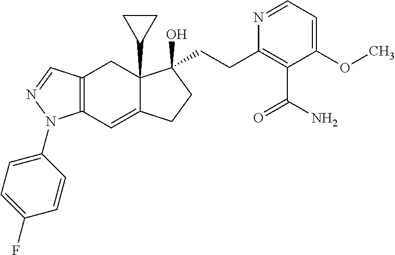

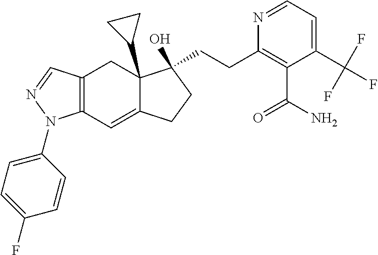

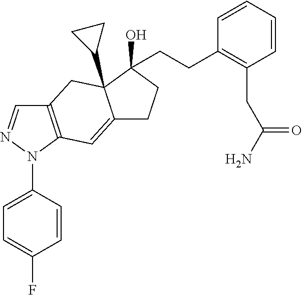

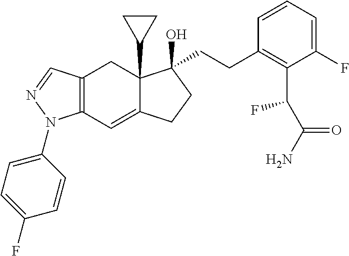

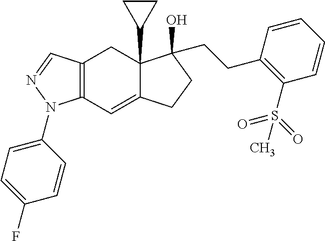

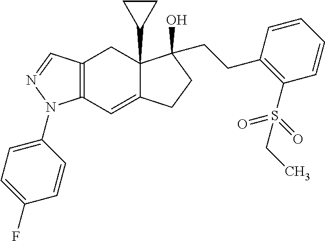

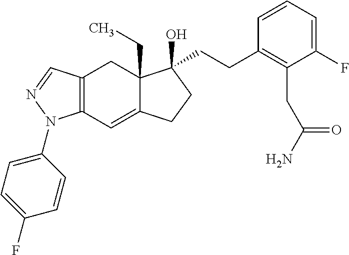

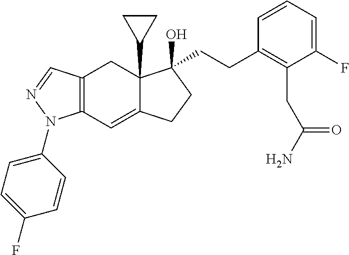

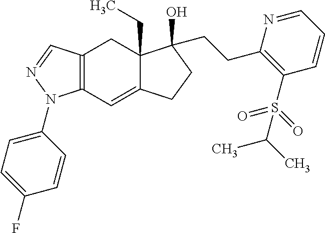

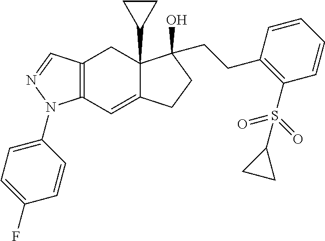

15. A pharmaceutical composition comprising a compound of claim 1 in combination with a pharmaceutically acceptable carrier.

16. A method for treating a glucocorticoid receptor mediated disease or condition in a mammalian patient in need of such treatment comprising administering to the patient a compound of claim 1 in an amount that is effective for treating the glucocorticoid receptor mediated disease or condition.

17. The method of claim 16 wherein the glucocorticoid receptor mediated disease or condition is selected from the group consisting of: tissue rejection, leukemias, lymphomas, Cushing's syndrome, acute adrenal insufficiency, congenital adrenal hyperplasia, rheumatic fever, polyarteritis nodosa, granulomatous polyarteritis, inhibition of myeloid cell lines, immune proliferation/apoptosis, HPA axis suppression and regulation, hypercortisolemia, stroke and spinal cord injury, hypercalcemia, hypergylcemia, acute adrenal insufficiency, chronic primary adrenal insufficiency, secondary adrenal insufficiency, congenital adrenal hyperplasia, cerebral edema, thrombocytopenia, Little's syndrome, obesity, metabolic syndrome, inflammatory bowel disease, systemic lupus erythematosus, polyartitis nodosa, Wegener's granulomatosis, giant cell arteritis, rheumatoid arthritis, juvenile rheumatoid arthritis, uveitis, hay fever, allergic rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis, cirrhosis, inflammatory scalp alopecia, panniculitis, psoriasis, discoid lupus erythematosus, inflamed cysts, atopic dermatitis, pyoderma gangrenosum, pemphigus vulgaris, buflous pemphigoid, systemic lupus erythematosus, dermatomyositis, herpes gestationis, eosinophilic fasciitis, relapsing polychondritis, inflammatory vasculitis, sarcoidosis, Sweet's disease, type I reactive leprosy, capillary hemangiomas, contact dermatitis, atopic dermatitis, lichen planus, exfoliative dermatitus, erythema nodosum, acne, hirsutism, toxic epidermal necrolysis, erythema multiform, cutaneous T-cell lymphoma, Human Immunodeficiency Virus (HIV), cell apoptosis, cancer, Kaposi's sarcoma, retinitis pigmentosa, cognitive performance, memory and learning enhancement, depression, addiction, mood disorders, chronic fatigue syndrome, schizophrenia, sleep disorders, and anxiety.

18. A method of selectively modulating the activation, repression, agonism or antagonism effect of the glucocorticoid receptor in a mammal comprising administering to the mammal a compound of claim 1 in an amount that is effective to modulate the glucocorticoid receptor.

19. Use of a compound of claim 1 for the manufacture of a medicament for the treatment of a glucocorticoid receptor mediated disease or condition in a mammalian patient in need of such treatment.
